SEQUENCE LISTING (8 sequences)

SEQ ID NO: 1 - DNA, 12 bp, Artificial sequence
  Synthetic oligonucleotide
ggatgttctcga

SEQ ID NO: 2 - RNA, 12 nt, Artificial sequence
  Synthetic oligonucleotide
ucgagaacaucc

SEQ ID NO: 3 - DNA, 16 bp, Artificial sequence
  Synthetic oligonucleotide
gcatgttctcacatta

SEQ ID NO: 4 - RNA, 16 nt, Artificial sequence
  Synthetic oligonucleotide
uaaugugagaacaugc

SEQ ID NO: 5 - DNA, 3160 bp, Homo sapiens
cctcccctcgcccggcgcggtcccgtccgcctctcgctcgcctcccgcctcccctcggtcttccgaggcgcccgggctcccggcgcggcggcggagggggcgggcaggccggcgggcggtgatgtggcaggactctttatgcgctgcggcaggatacgcgctcggcgctgggacgcgactgcgctcagttctctcctctcggaagctgcagccatgatggaagtttgagagttgagccgctgtgaggcgaggccgggctcaggcgagggagatgagagacggcggcggccgcggcccggagcccctctcagcgcctgtgagcagccgcgggggcagcgccctcggggagccggccggcctgcggcggcggcagcggcggcgtttctcgcctcctcttcgtcttttctaaccgtgcagcctcttcctcggcttctcctgaaagggaaggtggaagccgtgggctcgggcgggagccggctgaggcgcggcggcggcggcggcggcacctcccgctcctggagcgggggggagaagcggcggcggcggcggccgcggcggctgcagctccagggagggggtctgagtcgcctgtcaccatttccagggctgggaacgccggagagttggtctctccccttctactgcctccaacacggcggcggcggcggcggcacatccagggacccgggccggttttaaacctcccgtccgccgccgccgcaccccccgtggcccgggctccggaggccgccggcggaggcagccgttcggaggattattcgtcttctccccattccgctgccgccgctgccaggcctctggctgctgaggagaagcaggcccagtcgctgcaaccatccagcagccgccgcagcagccattacccggctgcggtccagagccaagcggcggcagagcgaggggcatcagctaccgccaagtccagagccatttccatcctgcagaagaagccccgccaccagcagcttctgccatctctctcctcctttttcttcagccacaggctcccagacatgacagccatcatcaaagagatcgttagcagaaacaaaaggagatatcaagaggatggattcgacttagacttgacctatatttatccaaacattattgctatgggatttcctgcagaaagacttgaaggcgtatacaggaacaatattgatgatgtagtaaggtttttggattcaaagcataaaaaccattacaagatatacaatctttgtgctgaaagacattatgacaccgccaaatttaattgcagagttgcacaatatccttttgaagaccataacccaccacagctagaacttatcaaacccttttgtgaagatcttgaccaatggctaagtgaagatgacaatcatgttgcagcaattcactgtaaagctggaaagggacgaactggtgtaatgatatgtgcatatttattacatcggggcaaatttttaaaggcacaagaggccctagatttctatggggaagtaaggaccagagacaaaaagggagtaactattcccagtcagaggcgctatgtgtattattatagctacctgttaaagaatcatctggattatagaccagtggcactgttgtttcacaagatgatgtttgaaactattccaatgttcagtggcggaacttgcaatcctcagtttgtggtctgccagctaaaggtgaagatatattcctccaattcaggacccacacgacgggaagacaagttcatgtactttgagttccctcagccgttacctgtgtgtggtgatatcaaagtagagttcttccacaaacagaacaagatgctaaaaaaggacaaaatgtttcacttttgggtaaatacattcttcataccaggaccagaggaaacctcagaaaaagtagaaaatggaagtctatgtgatcaagaaatcgatagcatttgcagtatagagcgtgcagataatgacaaggaatatctagtacttactttaacaaaaaatgatcttgacaaagcaaataaagacaaagccaaccgatacttttctccaaattttaaggtgaagctgtacttcacaaaaacagtagaggagccgtcaaatccagaggctagcagttcaacttctgtaacaccagatgttagtgacaatgaacctgatcattatagatattctgacaccactgactctgatccagagaatgaaccttttgatgaagatcagcatacacaaattacaaaagtctgaatttttttttatcaagagggataaaacaccatgaaaataaacttgaataaactgaaaatggacctttttttttttaatggcaataggacattgtgtcagattaccagttataggaacaattctcttttcctgaccaatcttgttttaccctatacatccacagggttttgacacttgttgtccagttgaaaaaaggttgtgtagctgtgtcatgtatatacctttttgtgtcaaaaggacatttaaaattcaattaggattaataaagatggcactttcccgttttattccagttttataaaaagtggagacagactgatgtgtatacgtaggaattttttccttttgtgttctgtcaccaactgaagtggctaaagagctttgtgatatactggttcacatcctacccctttgcacttgtggcaacagataagtttgcagttggctaagagaggtttccgaaaggttttgctaccattctaatgcatgtattcgggttagggcaatggaggggaatgctcagaaaggaaataattttatgctggactctggaccatataccatctccagctatttacacacacctttctttagcatgctacagttattaatctggacattcgaggaattggccgctgtcactgcttgttgtttgcgcatttttttttaaagcatattggtgctagaaaaggcagctaaaggaagtgaatctgtattggggtacaggaatgaaccttctgcaacatcttaagatccacaaatgaagggatataaaaataatgtcataggtaagaaacacagcaacaatgacttaaccatataaatgtggaggctatcaacaaagaatgggcttgaaacattataaaaattgacaatgatttattaaatatgttttctcaattgtaaaaaaaaaa

SEQ ID NO: 6 - DNA, 26 bp, Artificial sequence
  Primer
aatggctaagtgaagatgacaatcat

SEQ ID NO: 7 - DNA, 25 bp, Artificial sequence
  Primer
tgcacatatcattacaccagttcgt

SEQ ID NO: 8 - DNA, 30 bp, Artificial sequence
  Probe
ttgcagcaattcactgtaaagctggaaagg

(19) United States
(12) Patent Application Publication
(10) Pub. No.: US 2021/0254059 A1
(43) Pub. Date: Aug. 19, 2021

(54) LINKAGE MODIFIED OLIGOMERIC COMPOUNDS

(71) Applicant: Ionis Pharmaceuticals, Inc., Carlsbad, CA (US)

(72) Inventors: Michael T. Migawa, Carlsbad, CA (US); Eric E. Swayze, Encinitas, CA (US); Punit P. Seth, Carlsbad, CA (US)

(73) Assignee: Ionis Pharmaceuticals, Inc., Carlsbad, CA (US)

(21) Appl. No.: 16/973,661

(22) PCT Filed: Jun. 17, 2019
(86) PCT No.: PCT/US2019/037460

§ 371 Date: Dec. 9, 2020

Related U.S. Application Data
(60) Provisional application No. 62/686,628, filed on Jun. 18, 2018.

(51) Int. Cl. C12N 15/113; A61K 45/06

(52) U.S. Cl. CPC C12N 15/113; A61K 45/06; C12N 2310/315; C12N 2310/345; C12N 2310/351; C12N 2310/321; C12N 2310/3341; C12N 2310/341

(57) ABSTRACT

The present invention provides gapped oligomeric compounds comprising from 1 to about 3 internucleoside linkages having one of formulas I to XVI. In certain embodiments, inclusion of from 1 to about 3 internucleoside linkages of one of formulas I to XVI, improves selectivity for a target RNA relative to an off target RNA. In certain embodiments, the improved selectivity also provides an improved toxicity profile. Certain such oligomeric compounds are useful for hybridizing to a complementary nucleic acid, including but not limited, to nucleic acids in a cell. In certain embodiments, hybridization results in modulation of the amount of activity or expression of the target nucleic acid in a cell.

### FIELD OF THE INVENTION

The present invention pertains generally to chemically-modified oligonucleotides for use in research, diagnostics, and/or therapeutics.

### SEQUENCE LISTING

The present application is being filed along with a Sequence Listing in electronic format. The Sequence Listing is provided as a file entitled CHEM0097WOSEQ_ST25.txt, created Jun. 7, 2019 which is 8 Kb in size. The information in the electronic format of the sequence listing is incorporated herein by reference in its entirety.

### BACKGROUND OF THE INVENTION

Antisense compounds have been used to modulate target nucleic acids. Antisense compounds comprising a variety of chemical modifications and motifs have been reported. In certain instances, such compounds are useful as research tools, diagnostic reagents, and as therapeutic agents. In certain instances antisense compounds have been shown to modulate protein expression by binding to a target messenger RNA (mRNA) encoding the protein. In certain instances, such binding of an antisense compound to its target mRNA results in cleavage of the mRNA. Antisense compounds that modulate processing of a pre-mRNA have also been reported. Such antisense compounds alter splicing, interfere with polyadenlyation or prevent formation of the 5′-cap of a pre-mRNA.

Generally, the principle behind antisense technology is that an antisense compound hybridizes to a target nucleic acid and modulates gene expression activities or function, such as transcription or translation. The modulation of gene expression can be achieved by, for example, target degradation or occupancy-based inhibition. An example of modulation of RNA target function by degradation is RNase H-based degradation of the target RNA upon hybridization with a DNA-like antisense compound. Another example of modulation of gene expression by target degradation is RNA interference (RNAi). RNAi generally refers to antisense-mediated gene silencing involving the introduction of dsRNA leading to the sequence-specific reduction of targeted endogenous mRNA levels. An additional example of modulation of RNA target function by an occupancy-based mechanism is modulation of microRNA function. MicroRNAs are small non-coding RNAs that regulate the expression of protein-coding RNAs. The binding of an antisense compound to a microRNA prevents that microRNA from binding to its messenger RNA targets, and thus interferes with the function of the microRNA. Regardless of the specific mechanism, this sequence-specificity makes antisense compounds extremely attractive as tools for target validation and gene functionalization, as well as therapeutics to selectively modulate the expression of genes involved in the pathogenesis of malignancies and other diseases.

Antisense technology is an effective means for reducing the expression of one or more specific gene products and can therefore prove to be uniquely useful in a number of therapeutic, diagnostic, and research applications. Chemically modified nucleosides are routinely used for incorporation into antisense compounds to enhance one or more properties, such as nuclease resistance, pharmacokinetics or affinity for a target RNA. In 1998, the antisense compound, Vitravene® (fomivirsen; developed by Isis Pharmaceuticals Inc., Carlsbad, Calif.) was the first antisense drug to achieve marketing clearance from the U.S. Food and Drug Administration (FDA), and is currently a treatment of cytomegalovirus (CMV)-induced retinitis in AIDS patients.

New chemical modifications have improved the potency and efficacy of antisense compounds, uncovering the potential for oral delivery as well as enhancing subcutaneous administration, decreasing potential for side effects, and leading to improvements in patient convenience. Chemical modifications increasing potency of antisense compounds allow administration of lower doses, which reduces the potential for toxicity, as well as decreasing overall cost of therapy. Modifications increasing the resistance to degradation result in slower clearance from the body, allowing for less frequent dosing. Different types of chemical modifications can be combined in one compound to further optimize the compound's efficacy.

Targeting disease-causing gene sequences was first suggested more than thirty years ago (Belikova et al., Tet. Lett. 1967, 8(37), 3557-3562), and antisense activity was demonstrated in cell culture more than a decade later (Zamecnik et al., Proc. Natl. Acad. Sci. U.S.A. 1978, 75(1), 280-284). One advantage of antisense technology in the treatment of a disease or condition that stems from a disease-causing gene is that it is a direct genetic approach that has the ability to modulate (increase or decrease) the expression of specific disease-causing genes. Another advantage is that validation of a therapeutic target using antisense compounds results in direct and immediate discovery of the drug candidate; the antisense compound is the potential therapeutic agent.

Several nitrogen containing backbone modifications similar to the amides were evaluated as dimeric nucleosides (Sanghvi et al., Nucleosides Nucleotides 1997, 16, pp. 907-916). Peoc'h reported the synthesis of four methylene(methylimino) (MMI) linked oligodeoxyribonucleotide dimers modified at the 2′-position with fluoro and/or methoxy groups and their incorporation into different sequences (Peoc'h et al., Nucleosides, Nucleotides & Nucleic Acids, 23, pp. 411-438, 2004). Amino linkages have been synthesized and studied for enhanced cellular absorption (Saha el al., Tetrahedron Lett. 1993, 34, 6017-6020; De Mesmaeker el al., J. Bioorg. Med. Chem. Lett. 1994, 4, pp. 395-398; Caulfield et al, Bioorg. Med. Chem. Lett. 1993, 3, pp. 2771-2776). Other nitrogen containing backbones include oxime (Sanghvi et al., In Nucleosides and Nucleotides as Antitumor and Antiviral Agents; C. K. Chu and D. C. Baker Eds.: Plenum Press: New York, 1993, pp. 311-324), methyleneimino (ibid), methyleneoxy (methylimino) (MOMI) (ibid), methylene(dimethylhydrazo) (MDH) (Sanghvi et al., Collect. Czech. Chem. Commun. Special Issue 1993, 58, pp. 158-162), hydroxyl (methyliminomethylene) (HMIM) (Sanghvi et al., 11^{th} IRT Nucleosides & Nucleotides, Leuven, Belgium, Sep. 7-11, 1994 (poster presentation)), carbamate (Dabkowski et al., J Chem. Soc. Perkin Trans. 1 1994, pp. 817-829), oxyamide linkage (Burgess et al., J. Chem. Soc. Chem. Commun. 1994, pp. 915-916), N-substituted guanidine (Vandendrissche et al., J. Chem. Soc. 1993, pp. 1567-1575; Pannecouque et al., Tetrahedron 1994, 50, 7231-7246), urea (Kutterer et al., Bioorg. Med. Chem. Lett. 1994, 3, pp. 435-438) and thiourea linkages (Vandendrissche et al., J. Chem. Soc. 1993, pp. 1567-1575).

Synthesis of sulfur-containing backbone modifications, such as sulfonamide (McElroy et al., Bioorg. Med. Chem. Lett. 1994, 4, 1071-1076), sulfamoyl (Dewynter et al., Acad. Sci. 1992, 315, pp. 1675-1682), sulfonate (Huang et al., Synlett 1993, pp. 83-84), sulfide (Wang et al., Chin. Chem. Lett. 1993, 4, pp. 101-104; Huang et al., Synlett 1993, pp. 83-84; Kawai et al., Nucleic Acids Res. 1993, 21, pp. 1473-1479; Meng et al., J. Angew. Chem. Int. Ed. Engl. 1993, 32, pp. 729-731; Just el al. (1994), Synthesis and Hybridization Properties of DNA Oligomers Containing Sulfide-Linked Dinucleosides. In Carbohydrate Modifications in Antisense Research; Y. S. Sanghvi and P. D. Cook Eds. ACS Symposium Series 580; (pp. 52-65)), and sulfone linkages (Just el al. (1994), Synthesis and Hybridization Properties of DNA Oligomers Containing Sulfide-Linked Dinucleosides. In Carbohydrate Modifications in Antisense Research; Y. S. Sanghvi and P. D. Cook Eds. ACS Symposium Series 580; (pp. 62-65)) have been accomplished by several research groups.

Amide-linked dimers having one or two LNA nucleosides have been prepared and placed at internal positions within an oligomeric compound to determine their effects on Tm relative to a DNA/RNA duplex (Lauritsen et al., Chem. Commun., 2002, 530-532).

DNA or RNA containing oligonucleotides comprising alkylphosphonate internucleoside linkage backbone have been disclosed (see U.S. Pat. Nos. 5,264,423 and 5,286,717).

Oligomeric compounds have been prepared using Click chemistry wherein alkynyl phosphonate internucleoside linkages on an oligomeric compound attached to a solid support are converted into the 1,2,3-triazolylphosphonate internucleoside linkages and then cleaved from the solid support (Krishna et al., J. Am. Chem. Soc. 2012, 134(28), 11618-11631).

The synthesis of oligodeoxyribonucleotides containing a methyl phosphonate locked nucleic acid (LNA) thymine monomer has been described. The Tm values of the duplexes with their DNA or RNA complements have also been reported (see Lauritsen et al., Bioorg. Med. Chem. Lett. 2003, 13(2), 253-256).

DNA or RNA containing oligonucleotides comprising alkylphosphonate internucleoside linkage backbone have been disclosed (see U.S. Pat. Nos. 5,264,423 and 5,286,717).

A multitude of modified internucleoside linkages, including thioformacetal and amide-3 have been put into oligomeric compounds for Tm studies (Freier et al., Nucleic Acids Research, 1997, 25(22), 4429-4443).

Various dephosphono linkages (linkages without the phosphorus atom) modifications have been synthesized and studied for their antisense properties. Nonionic, achiral amide linkages were disclosed (Just et al., Synlett 1994, 137-139). A full account of the synthesis and properties of the five isomeric amide modifications was described (De Mesmaeker el al., (1994) Novel Backbone Replacements for Oligonucleotides, In Carbohydrate Modifications in Antisense Research; Y. S. Sanghvi and P. D. Cook Eds. ACS Symposium Series 580:24-39). The synthesis and incorporation of amide-3 internucleoside linkages into oligomers for various studies has been previously disclosed (Nina et al., JACS, 2005, 127, 6027-6038; Matt et al., Tetrahedron Letters, 1999, 40, 2899-2902; Druillennec et al., Bioorganic and Medicinal Chemistry Letters, 1999, 9, 627-632; Waldner et al., Bioorganic & Medicinal Chemistry Letters, 1996, 6(19), 2363-2366; and De Mesmaeker et al., Chem. Int. Ed. Engl., 1994, 33(2), 226-229).

The synthesis and incorporation of formacetal internucleoside linkages into oligomers for various studies has been previously disclosed (Kolarovi et al., JACS, 2009, 131, 14932-14978; Rozners et al., ChemBioChem, 1007, 8, 537-545 (note mixed 2′-OCH₃/formacetal oligos); and Mark Matteucci, Tetrahedron Letters, 1990, 17, 2385-2388).

Backbone substitution with formacetal and the related thioformacetal (Jones et al., J. Org. Chem., 58, pp. 2983-2991, 1993). Matteucci reported the synthesis of oligonucleotide analogs with one or more phosphodiester linkages that are replaced by a formacetal/ketal type linkage (U.S. Pat. No. 5,264,562 filed Apr. 24, 1991).

The synthesis and incorporation of thioformacetal (and alternate orientation, alt-thioformacetal, dimers only) internucleoside linkages into oligomers for various studies has been previously disclosed (Zhang et al., Bioorganic and Medicinal Chemistry Letters, 1999, 9, 319-322; and Ducharme et al., Tetrahedron Letters, 1995, 36(37), 6643-6646).

The synthesis and incorporation of glycine amide internucleoside linkages into oligomers for various studies has been previously disclosed (Bagmare et al., Tetrahedron, 2013, 69, 2010-2016; and Banerjee et al., Bioconjugate Chemistry, 2015, 26, 1737-1742).

The synthesis and incorporation of thioacetamido nucleic acid (TANA) internucleoside linkages into oligomers for various studies has been previously disclosed (Gogoi et al., Organic Letters, 2007, 9(14), 2697-2700; and Sharma et al., Nucleosides, Nucleotides and Nucleic Acids, 2015, 5(32), 256-272 (note LNA TANA LNA dimers). Gogoi et al. presented the synthesis of thioacetamido nucleic acids (TANA) backbone and thermal stability studies with complementary DNA and RNA sequences (Gogoi et al., Chem. Commun., 2006, pp. 2373-2375).

The synthesis of phosphoryl guanidine internucleoside linkages has been previously disclosed (Kupryushkin et al., Acta Naturae, 1014, 4(23), 116-118).

### SUMMARY OF THE INVENTION

Provided herein are oligomeric compounds comprising at least one internucleoside linking group having one of formulas I to XVI. In certain embodiments, oligomeric compounds are provided comprising a gapped oligomeric compound comprising a contiguous sequence of linked monomer subunits having a gap region located between a 5′-region and a 3′-region wherein the 5′ and 3′-regions each, independently, have from 2 to 8 contiguous RNA-like modified nucleosides that each adopt a 3′-endo conformational geometry when put into an oligomeric compound and the gap region has from 6 to 14 contiguous monomer subunits selected from β-D-2′-deoxyribonucleosides and modified nucleosides that are DNA like that each adopt a 2′-endo conformational geometry when put into an oligomeric compound and wherein at least one of the internucleoside linking groups in the gap region or linking the gap region and the 5′-region or the 3′-region has one of formulas I to XVI.

In certain embodiments, oligomeric compounds are provided comprising gapped oligomeric compounds that each comprise a contiguous sequence of linked monomer subunits having a 5′-region, a 3′-region and a gap region of from 6 to 14 contiguous β-D-2′-deoxyribonucleosides located between the 5′ and 3′-regions wherein the 5′ and 3′-regions each, independently, have from 2 to 8 contiguous modified nucleosides that are RNA-like that each adopt a 3′-endo conformational geometry when put into an oligomeric compound wherein each internucleoside linking group is, independently, a phosphodiester or a phosphorothioate internucleoside linking group providing that from 1 to about 3 internucleoside linking groups located in a gap junction and or the gap region is an internucleoside linking group having one of formulas I to XVI:

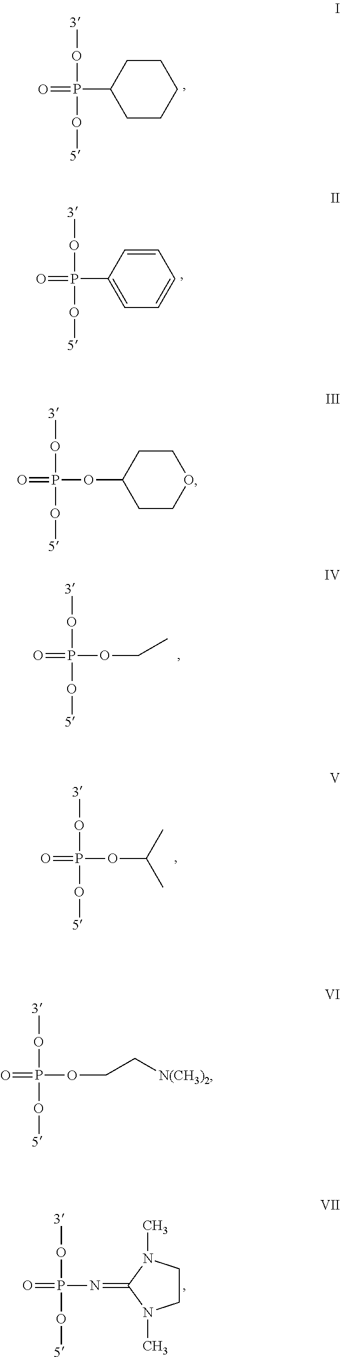

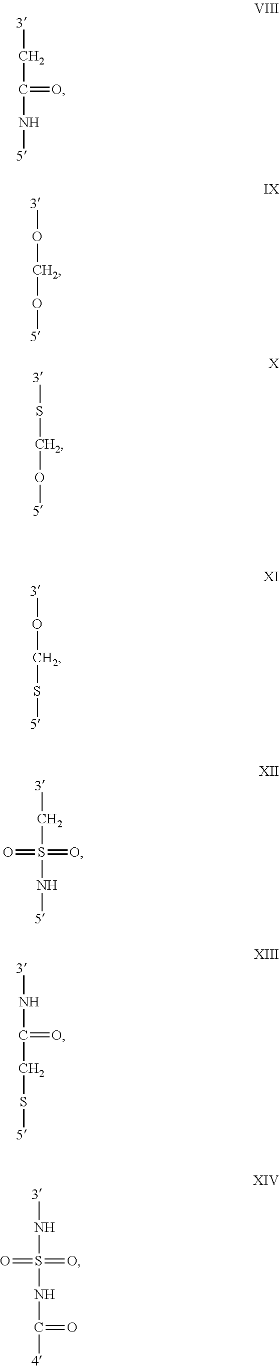

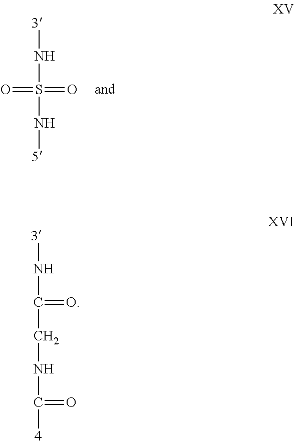

In certain embodiments, gapped oligomeric compounds are provided comprising from 12 to 24 monomer subunits. In certain embodiments, gapped oligomeric compounds are provided comprising from 14 to 20 monomer subunits. In certain embodiments, gapped oligomeric compounds are provided having 14 monomer subunits. In certain embodiments, gapped oligomeric compounds are provided having 16 monomer subunits. In certain embodiments, gapped oligomeric compounds are provided having 18 monomer subunits. In certain embodiments, gapped oligomeric compounds are provided having 20 monomer subunits.

In certain embodiments, the gap region has 10 contiguous monomer subunits and the 5′ and 3′-regions each, independently, have 2, 3 or 5 contiguous monomer subunits. In certain embodiments, the gap region has 10 contiguous monomer subunits and the 5′ and 3′-regions each have 5 contiguous monomer subunits. In certain embodiments, the gap region has 10 contiguous monomer subunits and the 5′ and 3′-regions each have 3 contiguous monomer subunits. In certain embodiments, the gap region has 10 contiguous monomer subunits and the 5′ and 3′-regions each have 2 contiguous monomer subunits.

In certain embodiments, gapped oligomeric compounds are provided comprising from 1 to about 3 internucleoside linking groups located in a gap junction and or the gap region having Formula I. In certain embodiments, gapped oligomeric compounds are provided comprising from 1 to about 3 internucleoside linking groups located in a gap junction and or the gap region having Formula II. In certain embodiments, gapped oligomeric compounds are provided comprising from 1 to about 3 internucleoside linking groups located in a gap junction and or the gap region having Formula III. In certain embodiments, gapped oligomeric compounds are provided comprising from 1 to about 3 internucleoside linking groups located in a gap junction and or the gap region having Formula IV. In certain embodiments, gapped oligomeric compounds are provided comprising from 1 to about 3 internucleoside linking groups located in a gap junction and or the gap region having Formula V. In certain embodiments, gapped oligomeric compounds are provided comprising from 1 to about 3 internucleoside linking groups located in a gap junction and or the gap region having Formula VI. In certain embodiments, gapped oligomeric compounds are provided comprising from 1 to about 3 internucleoside linking groups located in a gap junction and or the gap region having one of formulas IV, V or VI. In certain embodiments, gapped oligomeric compounds are provided comprising from 1 to about 3 internucleoside linking groups located in a gap junction and or the gap region having formula VII. In certain embodiments, gapped oligomeric compounds are provided comprising from 1 to about 3 internucleoside linking groups located in a gap junction and or the gap region having Formula VIII. In certain embodiments, gapped oligomeric compounds are provided comprising from 1 to about 3 internucleoside linking groups located in a gap junction and or the gap region having Formula IX. In certain embodiments, gapped oligomeric compounds are provided comprising from 1 to about 3 internucleoside linking groups located in a gap junction and or the gap region having Formula X. In certain embodiments, gapped oligomeric compounds are provided comprising from 1 to about 3 internucleoside linking groups located in a gap junction and or the gap region having Formula XI. In certain embodiments, gapped oligomeric compounds are provided comprising from 1 to about 3 internucleoside linking groups located in a gap junction and or the gap region having one of formulas VIII, IX, X and XI. In certain embodiments, gapped oligomeric compounds are provided comprising from 1 to about 3 internucleoside linking groups located in a gap junction and or the gap region having formula XV. In certain embodiments, gapped oligomeric compounds are provided comprising from 1 to about 3 internucleoside linking groups located in a gap junction and or the gap region having Formula XIII In certain embodiments, gapped oligomeric compounds are provided comprising from 1 to about 3 internucleoside linking groups located in a gap junction and or the gap region having Formula XIV. In certain embodiments, gapped oligomeric compounds are provided comprising from 1 to about 3 internucleoside linking groups located in a gap junction and or the gap region having Formula XVI. In certain embodiments, gapped oligomeric compounds are provided comprising from 1 to about 3 internucleoside linking groups located in a gap junction and or the gap region having one of formulas XIII, XIV and XVI.

In certain embodiments, oligomeric compounds are provided having 1 internucleoside linking group having one of formulas I to XVI. In certain embodiments, oligomeric compounds are provided having 2 internucleoside linking groups having one of formulas I to XVI. In certain embodiments, oligomeric compounds are provided having 3 internucleoside linking groups having one of formulas I to XVI. In certain embodiments, oligomeric compounds are provided having 2 or 3 contiguous internucleoside linking groups having one of formulas I to XVI.

In certain embodiments, oligomeric compounds are provided having 2 internucleoside linking groups having one of formulas I to XVI located between nucleosides 1 and 3, 2 and 4, 3 and 5 or 4 and 6 counting from the 5′ gap junction. In certain embodiments, oligomeric compounds are provided having 2 internucleoside linking groups having one of formulas I to XVI located between nucleosides 1 and 3 counting from the 5′ gap junction. In certain embodiments, oligomeric compounds are provided having 2 internucleoside linking groups having one of formulas I to XVI located between nucleosides 2 and 4 counting from the 5′ gap junction. In certain embodiments, oligomeric compounds are provided having 2 internucleoside linking groups having one of formulas I to XVI located between nucleosides 3 and 5 counting from the 5′ gap junction. In certain embodiments, oligomeric compounds are provided having 2 internucleoside linking groups having one of formulas I to XVI located between nucleosides 4 and 6 counting from the 5′ gap junction.

In certain embodiments, oligomeric compounds are provided having one internucleoside linking group having one of formulas I to XVI located between nucleosides 1 and 2, 2 and 3 or between nucleosides 3 and 4 counting from the 5′ gap junction. In certain embodiments, oligomeric compounds are provided having one internucleoside linking group having one of formulas I to XVI located between nucleosides 1 and 2 counting from the 5′ gap junction. In certain embodiments, oligomeric compounds are provided having one internucleoside linking group having one of formulas I to XVI located between nucleosides 2 and 3 counting from the 5′ gap junction. In certain embodiments, oligomeric compounds are provided having one internucleoside linking group having one of formulas I to XVI located between nucleosides 3 and 4 counting from the 5′ gap junction.

In certain embodiments, oligomeric compounds are provided wherein each internucleoside linking group having one of formulas I to XVI has the same formula.

In certain embodiments, the internucleoside linking groups in the 5′ and 3′-gap junctions are each, independently, a phosphodiester or a phosphorothioate internucleoside linking group. In certain embodiments, the internucleoside linking groups in the 5′ and 3′-gap junctions are each phosphodiester internucleoside linking groups. In certain embodiments, the internucleoside linking groups in the 5′ and 3′-gap junctions are each phosphorothioate internucleoside linking groups. In certain embodiments, oligomeric compounds are provided comprising an internucleoside linking group having one of formulas I to XVI located at the 5′-gap junction. In certain embodiments, oligomeric compounds are provided comprising an internucleoside linking group having one of formulas I to XVI located at the 3′-gap junction. In certain embodiments, each internucleoside linking group other than said internucleoside linking group having one of formulas I to XVI is a phosphodiester internucleoside linking group. In certain embodiments, each internucleoside linking group other than said an internucleoside linking group having one of formulas I to XVI is a phosphorothioate internucleoside linking group.

In certain embodiments, each monomer subunit comprises a nucleobase independently a purine, substituted purine, pyrimidine or substituted pyrimidine. In certain embodiments, each monomer subunit comprises a nucleobase independently selected from thymine, cytosine, 5-methyl-cytosine, adenine and guanine. In certain embodiments, each monomer subunit comprises a nucleobase independently selected from uracil, thymine, cytosine, 5-methylcytosine, adenine and guanine.

In certain embodiments, each modified nucleoside comprises a modified sugar moiety independently selected from a bicyclic nucleoside comprising a bicyclic furanosyl sugar moiety, a modified nucleoside comprising a furanosyl sugar moiety having at least one substituent group and a modified nucleoside comprising a sugar surrogate group. In certain embodiments, each modified nucleoside is, independently, selected from a bicyclic nucleoside comprising a bicyclic furanosyl sugar moiety having a bridging group between the 4′ and 2′ carbon atoms of the furanosyl ring independently selected from 4′-CH₂—O-2′, 4′-(CH₂)₂—O-2′, 4′-CH(CH₃)—O-2′, 4′-CH₂—N(CH₃)—O-2′, 4′-CH₂—C(H)(CH₃)-2′ and 4′-CH₂—C(═CH₂)-2′ and a modified nucleoside comprising a ribofuranosyl sugar moiety having at least a 2′-substituent group independently selected from F, OCH₃, O(CH₂)₂—OCH₃ and OCH₂C(═O)—N(H)CH₃. In certain embodiments, each of the modified nucleoside is, independently, selected from a bicyclic nucleoside comprising a bicyclic furanosyl sugar moiety having a 4′-CH₂—O-2′ or 4′-CH[(S)—(CH₃)]—O-2′ bridging group and a modified nucleoside comprising a ribofuranosyl sugar moiety having a 2′-O(CH₂)₂—OCH₃ substituent group. In certain embodiments, each of the modified nucleosides is, independently, selected from a bicyclic nucleoside comprising a bicyclic furanosyl sugar moiety having a 4′-CH[(S)—(CH₃)]—O-2′ bridging group and a modified nucleoside comprising a ribofuranosyl sugar moiety having a 2′-O(CH₂)₂—OCH₃ substituent group. In certain embodiments, each of the modified nucleoside is, independently, selected from a bicyclic nucleoside comprising a bicyclic furanosyl sugar moiety having a 4′-CH₂—O-2′ bridging group and a modified nucleoside comprising a ribofuranosyl sugar moiety having a 2′-O(CH₂)₂—OCH₃ substituent group. In certain embodiments, at least one of the modified nucleosides comprises a sugar surrogate. In certain embodiments, oligomeric compounds are provided wherein the modified nucleosides comprise 2 different types of sugar moieties.

In certain embodiments, oligomeric compounds are provided comprising at least one 5′ or 3′-terminal group. In certain embodiments, oligomeric compounds are provided having one optionally linked 5′ or 3′-conjugate group. In certain embodiments, oligomeric compounds are provided having one optionally linked 3′-conjugate group. In certain embodiments, oligomeric compounds are provided having one optionally linked 5′-conjugate group.

In certain embodiments, oligomeric compounds are provided comprising a conjugate group comprising a cell targeting moiety. In certain embodiments, the cell targeting moiety has the formula:

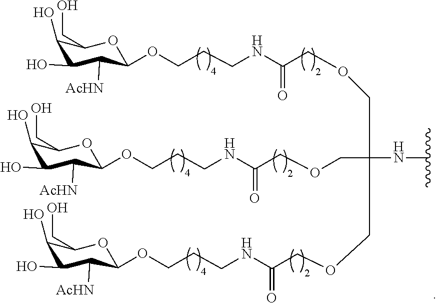

In certain embodiments, the cell targeting moiety has the formula:

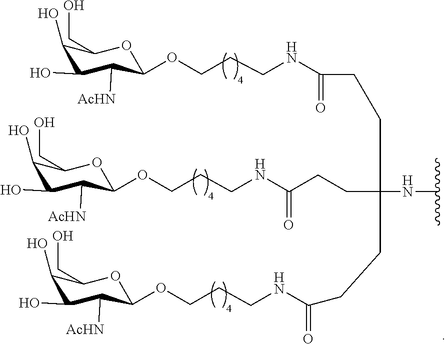

In certain embodiments, the cell targeting moiety has the formula:

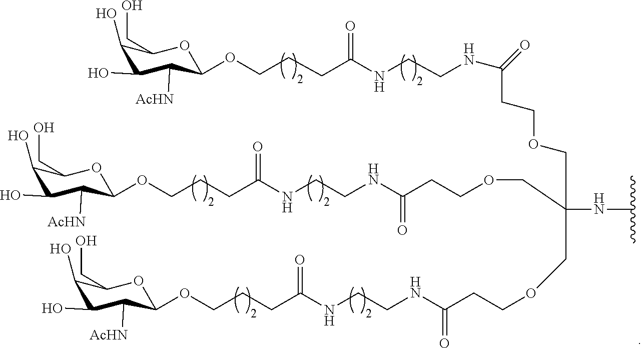

In certain embodiments, gapped oligomeric compounds are provided comprising a cell targeting moiety wherein the attachment of the cell targeting moiety to the oligomeric compound includes a conjugate linker and a cleavable moiety having one of the formulas:

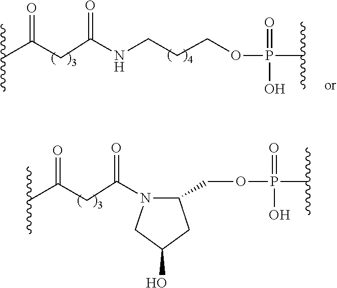

wherein the phosphate group is attached to the 3′ or 5′-terminal oxygen atom of the gapped oligomeric compound.

In certain embodiments, oligomeric compounds are provided comprising a conjugate group having the formula:

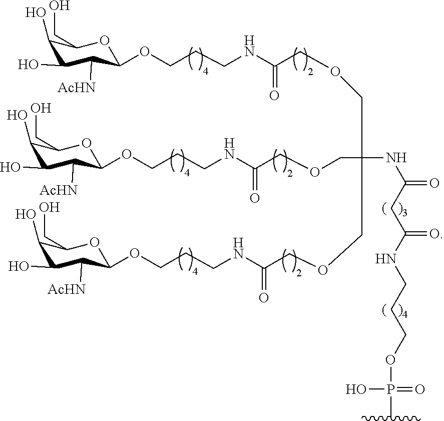

In certain embodiments, the conjugate group is attached to the 5′-terminal oxygen atom of the oligomeric compound.

In certain embodiments, oligomeric compounds are provided comprising a conjugate group having the formula:

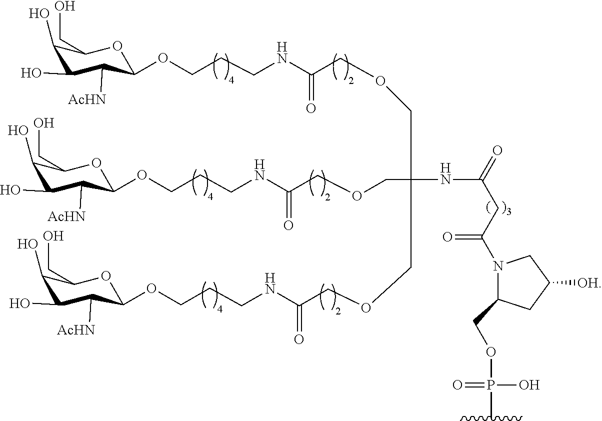

In certain embodiments, the conjugate group is attached to the 3′-terminal oxygen atom of the oligomeric compound.

In certain embodiments, methods of inhibiting gene expression are provided comprising contacting one or more cells, a tissue or an animal with a gapped oligomeric compound as provided herein wherein said oligomeric compound is complementary to a target RNA. In certain embodiments, the cells are in a human. In certain embodiments, the target RNA is human mRNA. In certain embodiments, the target RNA is cleaved thereby inhibiting its function.

In certain embodiments, methods of inhibiting gene expression are provided comprising contacting one or more cells or a tissue with a gapped oligomeric compound as provided herein.

In certain embodiments, gapped oligomeric compounds are provided for use in an in vivo method of inhibiting gene expression said method comprising contacting one or more cells, a tissue or an animal with a gapped oligomeric compound as provided herein.

In certain embodiments, gapped oligomeric compounds are provided for use in medical therapy.

### BRIEF DESCRIPTION OF THE FIGURE

FIG. 1 is a picture of a polyacrylamide gel showing cleavage patterns resulting from RNaseH 1 treatment of RNA/ASO duplexes (see Example 22 for complete details).

### DETAILED DESCRIPTION OF THE INVENTION

Provided herein are gapped oligomeric compounds that include from 1 to about 3 modified internucleoside linkages selected from formulas I to XVI. In certain embodiments, modified internucleoside linkages selected from formulas I to XVI are located in a gap junction and or in the gap region. In certain embodiments, modified internucleoside linkages selected from formulas I to XVI are located in the gap region and not in the gap junctions. In certain embodiments, the gapped oligomeric compounds further comprise an optionally linked conjugate group. The modified internucleoside linkages, having formulas I to XVI, are shown below:

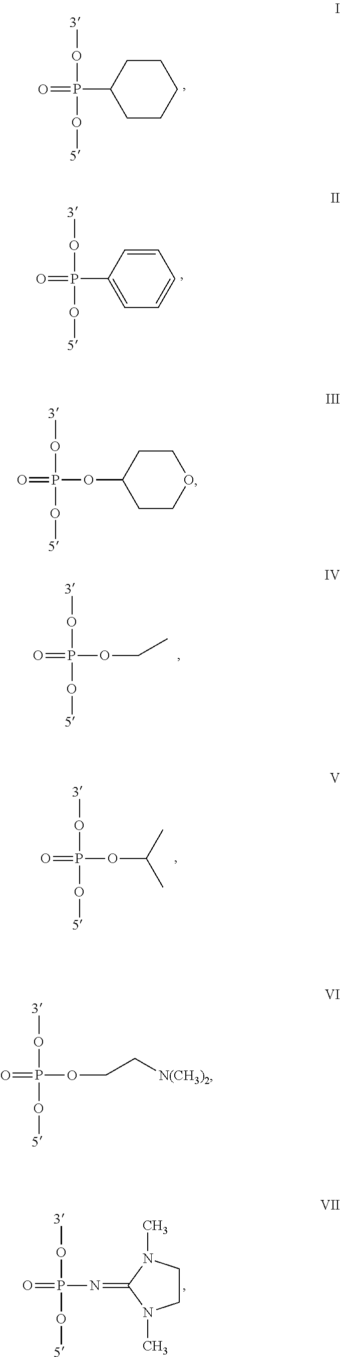

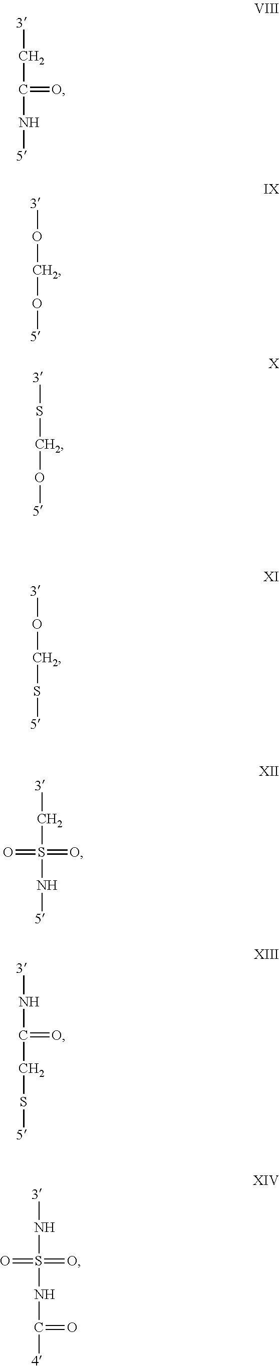

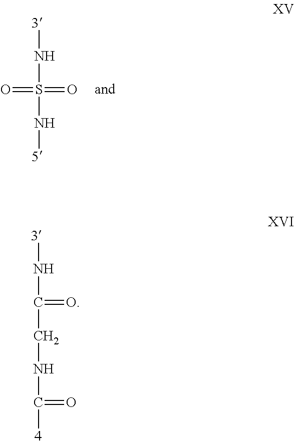

The present invention provides gapped oligomeric compounds comprising from 1 to about 3 internucleoside linkages having one of formulas I to XVI. In certain embodiments, inclusion from 1 to about 3 internucleoside linkages having of one of formulas I to XVI, improves selectivity for a target RNA relative to an off target RNA. In certain embodiments, the gapped oligomeric compound provides improved selectivity and an improved toxicity profile. In certain embodiments, oligomeric compounds provided herein have an enhanced therapeutic index. In certain embodiments, it is expected that the oligomeric compounds provided herein have improved potency for a target RNA. In certain embodiments, it is expected that the oligomeric compounds provided herein have enhanced stability to base exposure during synthesis. Certain such oligomeric compounds are useful for hybridizing to a complementary nucleic acid, including but not limited, to nucleic acids in a cell. In certain embodiments, hybridization results in modulation of the amount of activity or expression of the target nucleic acid in a cell.

In certain embodiments, gapped oligomeric compounds are provided comprising a contiguous sequence of linked monomer subunits having a 5′-region, a 3′-region and a gap region of from 6 to 14 contiguous β-D-2′-deoxyribonucleosides located between the 5′ and 3′-regions wherein the 5′ and 3′-regions each, independently, have from 2 to 8 contiguous modified nucleosides that are RNA-like that each adopt a 3′-endo conformational geometry when put into an oligomeric compound wherein from 1 to about 3 internucleoside linking groups located in a gap junction and or the gap region is a neutral internucleoside linking group having one of formulas I to XVI and the remainder of internucleoside linking groups are each independently, a phosphodiester or a phosphorothioate internucleoside linking group.

In certain embodiments, gapped oligomeric compounds are provided comprising two external regions (a 5′-region, a 3′-region) having from 6 to 14 contiguous β-D-2′-deoxy-ribonucleosides and an internal region further comprising at least one internucleoside linkage in a gap junction or the gap region selected from one of formulas I to XVI. The “gap junction” refers to the two internucleoside linkages on each end of the gap region separating the two external regions from the gap region. There is a 5′-gap junction and a 3′-gap junction defined by the directionality of the oligonucleotide which is routinely defined as reading from a 5′ to 3′ direction. For example the gapped oligonucleotide shown as a standard in multiple examples:

(SEQ ID NO.: 03, ISIS NO.: 558807)
5′-Gₖ^{m}CₖAₖTGTT^{m}CT^{m}CA^{m}CATₖTₖAₖ-3′

is a 3/10/3 gapmer with 3 modified nucleosides in each external region and 10 β-D-2′-deoxyribo-nucleosides in the gap region. This example of a gapped oligomeric compound comprises a 5′-gap junction as underlined between the Aₖ and T of AₖT and a 3′-gap junction as underlined between the A and Tₖ of A_{T}k. The internucleoside linkages in each of these gap junctions is a phosphorothioate for this gapped oligomeric compound.

The gapped oligomeric compounds provided herein have at least one internucleoside linkage selected from formulas I to XVI. In certain embodiments, gapped oligomeric compounds are provided comprising 1 internucleoside linkage selected from formulas I to XVI located in a gap junction. In certain embodiments, gapped oligomeric compounds are provided having from 1 to 3 internucleoside linkages selected from formulas I to XVI which are located in the gap region (not in a gap junction). In certain embodiments, gapped oligomeric compounds are provided having from 2 to 3 internucleoside linkages wherein 1 is located in a gap junction and 1 or 2 are located in the gap region. In certain embodiments, gapped oligomeric compounds are provided having a single or two contiguous internucleoside linkages that are the same (when two) having one of formulas I to XVI, and are located in the gap and not the gap junction.

In certain embodiments, gapped oligomeric compounds as provided herein are described in the shorthand E₅/G/E₃ wherein the “E₅” is the external region at the 5′-end, “G” is the gap region and “E₃” is the external region at the 3′-end. In certain embodiments, gapped oligomeric compounds are provided comprising a 2/10/2 motif. In certain embodiments, gapped oligomeric compounds are provided comprising a 3/10/3 motif. In certain embodiments, gapped oligomeric compounds are provided comprising a 5/10/5 motif. In certain embodiments, the modified nucleosides in the external regions are bicyclic modified nucleosides. In certain embodiments, the modified nucleosides in the external regions each comprise a 2′-substituent group selected from F, OCH₃, O(CH₂)₂—OCH₃ and OCH₂C(═O)—N(H)CH₃. In certain embodiments, the modified nucleosides in the external regions are a mixture of bicyclic modified nucleosides and modified nucleosides comprising at least one substituent group. In certain embodiments, the modified nucleosides in the external regions are a mixture of bicyclic modified nucleosides comprising a bridging group selected from 4′-CH₂—O-2′, 4′-(CH₂)₂—O-2′, 4′-CH(CH₃)—O-2′, 4′-CH₂—N(CH₃)—O-2′, 4′-CH₂—C(H)(CH₃)-2′ and 4′-CH₂—C(═CH₂)-2′ and modified nucleosides comprising a 2′-substituent group selected from F, OCH₃, O(CH₂)₂—OCH₃ and OCH₂C(═O)—N(H)CH₃. In certain embodiments, the modified nucleosides in the external regions are a mixture of bicyclic modified nucleosides comprising a bridging group selected from 4′-CH₂—O-2′ or 4′-CH[(S)—(CH₃)]—O-2′ and modified nucleosides comprising a 2′-substituent group selected from F, OCH₃, O(CH₂)₂—OCH₃ and OCH₂C(═O)—N(H)CH₃. In certain embodiments, the modified nucleosides in the external regions are a mixture of bicyclic modified nucleosides comprising a bridging group selected from 4′-CH[(S)—(CH₃)]—O-2′ and modified nucleosides comprising a 2′-O(CH₂)₂—OCH₃ substituent group. In certain embodiments, each modified nucleoside in each external region is a bicyclic modified nucleoside comprising a 4′-CH[(S)—(CH₃)]—O-2′ bridging group. In certain embodiments, each modified nucleoside in each external region is a 2′-O(CH₂)₂—OCH₃ modified nucleoside.

In certain embodiments, gapped oligomeric compounds are provided comprising a 2/10/2, 3/10/3, or 5/10/5 motif wherein each modified nucleoside in each external region is, independently, a bicyclic modified nucleoside comprising a 4′-CH[(S)—(CH₃)]—O-2′ bridging group or a modified nucleoside comprising a 2′-O(CH₂)₂—OCH₃ substituent group having a single modified internucleoside linkage having one of formulas I to XVI located between nucleosides 2 and 3 or between nucleosides 3 and 4 counting from the 5′ gap junction. In certain embodiments, gapped oligomeric compounds are provided comprising a 2/10/2, 3/10/3 or 5/10/5 motif wherein each modified nucleoside in each external region is, independently, a bicyclic modified nucleoside comprising a 4′-CH[(S)—(CH₃)]—O-2′ bridging group or a modified nucleoside comprising a 2′-O(CH₂)₂—OCH₃ substituent group having 2 modified internucleoside linkages having one of formulas I to XVI located between nucleosides 1 and 3, 2 and 4, 3 and 5 or 4 and 6 counting from the 5′ gap junction. In certain embodiments, each modified internucleoside linkage is the same. In certain embodiments, the gapped oligomeric compound is further functionalized by addition of a conjugate group.

In certain embodiments, gapped oligomeric compounds are provided comprising a 2/10/2, 3/10/3 or 5/10/5 motif wherein each modified nucleoside in each external region is a bicyclic modified nucleoside comprising a 4′-CH[(S)—(CH₃)]—O-2′ bridging group having a single modified internucleoside linkage having one of formulas I to XVI located between nucleosides 2 and 3 or between nucleosides 3 and 4 counting from the 5′ gap junction. In certain embodiments, gapped oligomeric compounds are provided comprising a 2/10/2, 3/10/3 or 5/10/5 motif wherein each modified nucleoside in each external region is a bicyclic modified nucleoside comprising a 4′-CH[(S)—(CH₃)]—O-2′ bridging group having 2 modified internucleoside linkages having one of formulas I to XVI located between nucleosides 1 and 3, 2 and 4, 3 and 5 or 4 and 6 counting from the 5′ gap junction. In certain embodiments, each modified internucleoside linkage is the same. In certain embodiments, the gapped oligomeric compound is further functionalized by addition of a conjugate group.

In certain embodiments, gapped oligomeric compounds are provided comprising a 2/10/2, 3/10/3 or 5/10/5 motif wherein each modified nucleoside in each external region is a modified nucleoside comprising a 2′-O(CH₂)₂—OCH₃ substituent group having a single modified internucleoside linkage having one of formulas I to XVI located between nucleosides 2 and 3 or between nucleosides 3 and 4 counting from the 5′ gap junction. In certain embodiments, gapped oligomeric compounds are provided comprising a 2/10/2, 3/10/3 or 5/10/5 motif wherein each a modified nucleoside in each external region comprises a 2′-O(CH₂)₂—OCH₃ substituent group having 2 modified internucleoside linkages having one of formulas I to XVI located between nucleosides 1 and 3, 2 and 4, 3 and 5 or 4 and 6 counting from the 5′ gap junction. In certain embodiments, each modified internucleoside linkage is the same. In certain embodiments, the gapped oligomeric compound is further functionalized by addition of a conjugate group.

In certain embodiments, a linkage unmodified gapped oligomeric compound of interest is identified and then a series of identical gapped oligomeric compounds are prepared having a single modified internucleoside linking group selected from one of formulas I to XVI walked from the 5′-gap junction to the 3′-gap junction across the gap region. If there are 10 monomer subunits in the gap then there will be 11 oligomeric compounds prepared having the selected modified internucleoside linking group having one of formulas I to XVI located at a different position in each of the oligomeric compounds which are subsequently assayed in one or more assays as illustrated herein to determine the lead from each series.

In certain embodiments, a linkage unmodified gapped oligomeric compound of interest is identified and then a series of identical gapped oligomeric compounds are prepared having 2 modified internucleoside linking groups selected from one of formulas I to XVI walked from the 5′-gap junction to the 3′-gap junction across the gap region wherein the two modified internucleoside linkages are contiguous. If there are 10 monomer subunits in the gap then there will be 10 oligomeric compounds prepared having the selected modified internucleoside linking groups having one of formulas I to XVI located at a different positions in each of the oligomeric compounds which are subsequently assayed in one or more assays as illustrated herein to determine the lead from each series.

It is to be understood that both the foregoing general description and the following detailed description are exemplary and explanatory only and are not restrictive. Herein, the use of the singular includes the plural unless specifically stated otherwise. As used herein, the use of “or” means “and/or” unless stated otherwise. Furthermore, the use of the term “including” as well as other forms, such as “includes” and “included”, is not limiting. Also, terms such as “element” or “component” encompass both elements and components comprising one unit and elements and components that comprise more than one subunit, unless specifically stated otherwise.

The section headings used herein are for organizational purposes only and are not to be construed as limiting the subject matter described. All documents, or portions of documents, cited in this application, including, but not limited to, patents, patent applications, articles, books, and treatises, are hereby expressly incorporated-by-reference for the portions of the document discussed herein, as well as in their entirety.

### Definitions

Unless specific definitions are provided, the nomenclature used in connection with, and the procedures and techniques of, analytical chemistry, synthetic organic chemistry, and medicinal and pharmaceutical chemistry described herein are those well-known and commonly used in the art. Where permitted, all patents, applications, published applications and other publications and other data referred to throughout in the disclosure are incorporated by reference herein in their entirety.

Unless otherwise indicated, the following terms have the following meanings:

As used herein, “2′-deoxynucleoside” means a nucleoside comprising 2′-H(H) furanosyl sugar moiety, as found in naturally occurring deoxyribonucleic acids (DNA). In certain embodiments, a 2′-deoxynucleoside may comprise a modified nucleobase or may comprise an RNA nucleobase (uracil).

As used herein, “2′-substituted nucleoside” or “2-modified nucleoside” means a nucleoside comprising a 2′-substituted or 2′-modified sugar moiety. As used herein, “2′-substituted” or “2-modified” in reference to a sugar moiety means a sugar moiety comprising at least one 2′-substituent group other than H or OH.

As used herein, “Antisense activity” means any detectable and/or measurable change attributable to the hybridization of an antisense compound to its target nucleic acid. In certain embodiments, antisense activity is a decrease in the amount or expression of a target nucleic acid or protein encoded by such target nucleic acid compared to target nucleic acid levels or target protein levels in the absence of the antisense compound. In certain embodiments, antisense activity is a change in splicing of a pre-mRNA nucleic acid target. In certain embodiments, antisense activity is an increase in the amount or expression of a target nucleic acid or protein encoded by such target nucleic acid compared to target nucleic acid levels or target protein levels in the absence of the antisense compound.

As used herein, “Antisense compound” means a compound comprising an antisense oligonucleotide and optionally one or more additional features, such as a conjugate group or terminal group.

As used herein, “Antisense oligonucleotide” means an oligonucleotide that (1) has a nucleobase sequence that is at least partially complementary to a target nucleic acid and that (2) is capable of producing an antisense activity in a cell or animal.

As used herein, “Ameliorate” in reference to a treatment means improvement in at least one symptom relative to the same symptom in the absence of the treatment. In certain embodiments, amelioration is the reduction in the severity or frequency of a symptom or the delayed onset or slowing of progression in the severity or frequency of a symptom.

As used herein, “Bicyclic nucleoside” or “BNA” means a nucleoside comprising a bicyclic sugar moiety. As used herein, “bicyclic sugar” or “bicyclic sugar moiety” means a modified sugar moiety comprising two rings, wherein the second ring is formed via a bridge or bridging group connecting two of the atoms in the first ring thereby forming a bicyclic structure. In certain embodiments, the first ring of the bicyclic sugar moiety is a furanosyl moiety. In certain embodiments, the bicyclic sugar moiety does not comprise a furanosyl moiety.

As used herein, “Branching group” means a group of atoms having at least 3 positions that are capable of forming covalent linkages to at least 3 groups. In certain embodiments, a branching group provides a plurality of reactive sites for connecting tethered ligands to an oligonucleotide via a conjugate linker and/or a cleavable moiety.

As used herein, “Cell-targeting moiety” means a conjugate group or portion of a conjugate group that is capable of binding to a particular cell type or particular cell types.

As used herein, “Cleavable moiety” means a bond or group of atoms that is cleaved under physiological conditions, for example, inside a cell, an animal, or a human.

As used herein, “Complementary” in reference to an oligonucleotide means that at least 70% of the nucleobases of such oligonucleotide or one or more regions thereof and the nucleobases of another nucleic acid or one or more regions thereof are capable of hydrogen bonding with one another when the nucleobase sequence of the oligonucleotide and the other nucleic acid are aligned in opposing directions. Complementary nucleobases means nucleobases that are capable of forming hydrogen bonds with one another. Complementary nucleobase pairs include, but unless otherwise specific are not limited to, adenine (A) and thymine (T), adenine (A) and uracil (U), cytosine (C) and guanine (G), 5-methyl cytosine (^{m}C) and guanine (G). Complementary oligonucleotides and/or nucleic acids need not have nucleobase complementarity at each nucleoside. Rather, some mismatches are tolerated. As used herein, “fully complementary” or “100% complementary” in reference to oligonucleotides means that such oligonucleotides are complementary to another oligonucleotide or nucleic acid at each nucleoside of the oligonucleotide.

As used herein, “Conjugate group” means a group of atoms that is directly or indirectly attached to an oligonucleotide. Conjugate groups include a conjugate group and a conjugate linker that attaches the conjugate group to the oligonucleotide wherein the attachment may include a cleavable moiety.

As used herein, “Conjugate linker” means a group of atoms comprising at least one bond that connects a conjugate group to an oligonucleotide wherein the attachment may include a cleavable moiety.

As used herein, “Contiguous” in the context of an oligonucleotide refers to nucleosides, nucleobases, sugar moieties, or internucleoside linkages that are immediately adjacent to each other. For example, “contiguous nucleobases” means nucleobases that are immediately adjacent to each other in a sequence.

As used herein, “Duplex” means two oligomeric compounds that are paired. In certain embodiments, the two oligomeric compounds are paired via hybridization of complementary nucleobases.

As used herein, “Extra-hepatic cell type” means a cell type that is not a hepatocyte.

As used herein, “Extra-hepatic nucleic acid target” means a target nucleic acid that is expressed in tissues other than liver. In certain embodiments, extra-hepatic nucleic acid targets are not expressed in the liver or not expressed in the liver at a significant level. In certain embodiments, extra-hepatic nucleic acid targets are expressed outside the liver and also in the liver.

As used herein, “Extra-hepatic tissue” means a tissue other than liver.

As used herein, “Fully modified” in reference to a modified oligonucleotide means a modified oligonucleotide in which each sugar moiety is modified. “Uniformly modified” in reference to a modified oligonucleotide means a fully modified oligonucleotide in which each sugar moiety is the same. For example, the nucleosides of a uniformly modified oligonucleotide can each have a 2′-MOE modification but different nucleobase modifications, and the internucleoside linkages may be different.

As used herein, “Gapmer” means an antisense oligonucleotide comprising an internal region having a plurality of nucleosides that support RNase H cleavage positioned between external regions having one or more nucleosides, wherein the nucleosides comprising the internal region are chemically distinct from the nucleoside or nucleosides comprising the external regions. The internal region may be referred to as the “gap” and the external regions may be referred to as the “wings.”

As used herein, “Hybridization” means the pairing or annealing of complementary oligonucleotides and/or nucleic acids. While not limited to a particular mechanism, the most common mechanism of hybridization involves hydrogen bonding, which may be Watson-Crick, Hoogsteen or reversed Hoogsteen hydrogen bonding, between complementary nucleobases.

As used herein, “Inhibiting the expression or activity” refers to a reduction or blockade of the expression or activity relative to the expression of activity in an untreated or control sample and does not necessarily indicate a total elimination of expression or activity.

As used herein, “Internucleoside linkage” or “internucleoside linking group” means a group or bond that forms a covalent linkage between adjacent nucleosides in an oligonucleotide. As used herein “modified internucleoside linkage” means any internucleoside linkage other than a naturally occurring, phosphate internucleoside linkage. Non-phosphodiester linkages are referred to herein as modified internucleoside linkages. “Phosphorothioate linkage” means a modified phosphodiester linkage in which one of the non-bridging oxygen atoms is replaced with a sulfur atom. A phosphorothioate internucleoside linkage is a modified internucleoside linkage. Modified internucleoside linkages include linkages that comprise abasic nucleosides. As used herein, “abasic nucleoside” means a sugar moiety in an oligonucleotide or oligomeric compound that is not directly connected to a nucleobase. In certain embodiments, an abasic nucleoside is adjacent to one or two nucleosides in an oligonucleotide.

As used herein, “Lipophilic group” or “lipophilic” in reference to a chemical group means a group of atoms that is more soluble in lipids or organic solvents than in water and/or has a higher affinity for lipids than for water. In certain embodiments, lipophilic groups comprise a lipid. As used herein “lipid” means a molecule that is not soluble in water or is less soluble in water than in organic solvents. In certain embodiments, compounds of the present invention comprise lipids selected from saturated or unsaturated fatty acids, steroids, fat soluble vitamins, phospholipids, sphingolipids, hydrocarbons, mono-, di-, and tri-glycerides, and synthetic derivatives thereof.

As used herein the term “monomer subunit” is meant to include all manner of monomers that are amenable to oligomer synthesis. In general a monomer subunit includes at least a sugar moiety or modified sugar moiety having at least two reactive sites that can form linkages to further monomer subunits. Essentially all monomer subunits include a nucleobase that is hybridizable to a complementary site on a nucleic acid target. Reactive sites on monomer subunits located on the termini of an oligomeric compound can be protected or unprotected (generally OH) or can form an attachment to a terminal group (conjugate or other group). Monomer subunits include, without limitation, nucleosides and modified nucleosides. In certain embodiments, monomer subunits include nucleosides such as β-D-ribonucleosides and β-D-2′-deoxyribnucleosides and modified nucleosides including but not limited to substituted nucleosides (such as 2′, 5′ and bis substituted nucleosides), 4′-S-modified nucleosides (such as 4′-S-ribonucleosides, 4′-S-2′-deoxyribonucleosides and 4′-S-2′-substituted ribonucleosides), bicyclic modified nucleosides (such as bicyclic nucleosides wherein the sugar moiety has a 2′-O—CHRₐ-4′ bridging group, wherein Ra is H, alkyl or substituted alkyl), other modified nucleosides and nucleosides having sugar surrogates.

As used herein, “Non-bicyclic modified sugar” or “non-bicyclic modified sugar moiety” means a modified sugar moiety that comprises a modification, such as a substituent, that does not form a bridge between two atoms of the sugar to form a second ring.

As used herein, “Linked nucleosides” are nucleosides that are connected in a continuous sequence (i.e. no additional nucleosides are present between those that are linked).

As used herein, “Mismatch” or “non-complementary” means a nucleobase of a first oligonucleotide that is not complementary with the corresponding nucleobase of a second oligonucleotide or target nucleic acid when the first and second oligomeric compound are aligned.

As used herein, “MOE” means methoxyethoxy. “2′-MOE” means a —OCH₂CH₂OCH₃ group at the 2′ position of a furanosyl ring.

As used herein, “Motif” means the pattern of unmodified and/or modified sugar moieties, nucleobases, and/or internucleoside linkages, in an oligonucleotide.

As used herein, “Multi-tissue disease or condition” means a disease or condition affects or is effected by more than one tissue. In treating a multi-tissue disease or condition, it is desirable to affect more than one tissue type. In certain embodiments, treatment of disease or condition may be enhanced by treating the disease or condition in multiple tissues. For example, in certain embodiments, a disease or condition may manifest itself in the liver tissue and the muscle tissue. In certain embodiments, treating the disease or condition in the liver tissue and the muscle tissue will be more effective than treating the disease in either the liver tissue or the muscle tissue.

As used herein, “Naturally occurring” means found in nature.

As used herein, “Nucleobase” means an unmodified nucleobase or a modified nucleobase. As used herein an “unmodified nucleobase” is adenine (A), thymine (T), cytosine (C), uracil (U), or guanine (G). As used herein, a “modified nucleobase” is a group of atoms other than unmodified A, T, C, U, or G capable of pairing with at least one unmodified nucleobase. Oligomeric compounds are most often prepared having nucleobases selected from adenine, guanine, thymine, cytosine, 5′-methyl cytosine and uracil. The optionally protected nucleobases commonly used for the synthesis of oligomeric compounds are 6-N-benzoyladenine, 2-N-isobutyrylguanine, 4-N-benzoylcytosine, 5′-methyl-4-N-benzoylcytosine, thymine and uracil.

As used herein, “Nucleoside” means a compound comprising a nucleobase and a sugar moiety. The nucleobase and sugar moiety are each, independently, unmodified or modified. As used herein, “modified nucleoside” means a nucleoside comprising a modified nucleobase and/or a modified sugar moiety. Modified nucleosides include abasic nucleosides, which lack a nucleobase.

As used herein, “Oligomeric compound” means a compound consisting of an oligonucleotide and optionally one or more additional features, such as a conjugate group or other terminal group.

As used herein, “Oligonucleotide” means a strand of linked nucleosides connected via internucleoside linkages, wherein each nucleoside and internucleoside linkage may be modified or unmodified. Unless otherwise indicated, oligonucleotides consist of 8-50 linked nucleosides. As used herein, “modified oligonucleotide” means an oligonucleotide, wherein at least one nucleoside or internucleoside linkage is modified. As used herein, “unmodified oligonucleotide” means an oligonucleotide that does not comprise any nucleoside modifications or internucleoside modifications.

As used herein, “Pharmaceutically acceptable carrier or diluent” means any substance suitable for use in administering to an animal. Certain such carriers enable pharmaceutical compositions to be formulated as, for example, tablets, pills, dragees, capsules, liquids, gels, syrups, slurries, suspension and lozenges for the oral ingestion by a subject. In certain embodiments, a pharmaceutically acceptable carrier or diluent is sterile water; sterile saline; or sterile buffer solution.

As used herein, “Pharmaceutically acceptable salts” means physiologically and pharmaceutically acceptable salts of compounds, such as oligomeric compounds, i.e., salts that retain the desired biological activity of the parent compound and do not impart undesired toxicological effects thereto.

As used herein, “Pharmaceutical composition” means a mixture of substances suitable for administering to a subject. For example, a pharmaceutical composition may comprise an antisense compound and a sterile aqueous solution. In certain embodiments, a pharmaceutical composition shows activity in free uptake assay in certain cell lines.

As used herein, “Phosphorus moiety” means a group of atoms comprising a phosphorus atom. In certain embodiments, a phosphorus moiety comprises a mono-, di-, or tri-phosphate, or phosphorothioate.

As used herein, “Prodrug” means a therapeutic agent in a form outside the body that is converted to a different form within the body or cells thereof. Typically conversion of a prodrug within the body is facilitated by the action of an enzymes (e.g., endogenous or viral enzyme) or chemicals present in cells or tissues and/or by physiologic conditions.

As used herein, “RNAi compound” means an antisense compound that acts, at least in part, through RISC or Ago2 to modulate a target nucleic acid and/or protein encoded by a target nucleic acid. RNAi compounds include, but are not limited to double-stranded siRNA, single-stranded RNA (ssRNA), and microRNA, including microRNA mimics. In certain embodiments, an RNAi compound modulates the amount, activity, and/or splicing of a target nucleic acid. The term RNAi compound excludes antisense oligonucleotides that act through RNase H.

As used herein, “RNA-like nucleoside” means a modified nucleoside other than a β-D-ribose nucleoside that provides an A-form (northern) duplex when incorporated into an oligomeric compound and duplexed with a complementary RNA. RNA-like nucleosides are used as replacements for RNA nucleosides in oligomeric compounds to enhance one or more properties such as, for example, nuclease resistance and or hybridization affinity. RNA-like nucleosides include, but are not limited to modified furanosyl nucleosides that adopt a 3′-endo conformational geometry when put into an oligomeric compound. RNA-like nucleosides also include RNA surrogates such as F-HNA. RNA-like nucleosides include but are not limited to modified nucleosides comprising a 2′-substituent group selected from F, O(CH₂)₂OCH₃ (MOE) and OCH₃. RNA-like nucleosides also include but are not limited to modified nucleosides comprising bicyclic furanosyl sugar moiety comprising a 4′-CH₂—O-2′, 4′-(CH₂)₂—O-2′, 4′-C(H)[(R)—CH₃]—O-2′ or 4′-C(H)[(S)—CH₃]—O-2′ bridging group.

As used herein, “Single-stranded” in reference to an oligomeric compound means such a compound that is not paired with a second oligomeric compound to form a duplex. “Self-complementary” in reference to an oligonucleotide means an oligonucleotide that at least partially hybridizes to itself. A compound consisting of one oligomeric compound, wherein the oligonucleotide of the oligomeric compound is self-complementary, is a single-stranded compound. A single-stranded antisense or oligomeric compound may be capable of binding to a complementary oligomeric compound to form a duplex, in which case it would no longer be single-stranded.

As used herein, “Standard cell assay” means the assay described in Example 1 and reasonable variations thereof.

As used herein, “Standard in vivo experiment” means the procedure described in Example 5 and reasonable variations thereof.

As used herein, “Sugar moiety” means an unmodified sugar moiety or a modified sugar moiety. As used herein, “unmodified sugar moiety” means a 2′-OH(H) furanosyl moiety, as found in RNA (an “unmodified RNA sugar moiety”), or a 2′-H(H) moiety, as found in DNA (an “unmodified DNA sugar moiety”). Unmodified sugar moieties have one hydrogen at each of the 1′, 3′, and 4′ positions, an oxygen at the 3′ position, and two hydrogens at the 5′ position. As used herein, “modified sugar moiety” or “modified sugar” means a modified furanosyl sugar moiety or a sugar surrogate. As used herein, modified furanosyl sugar moiety means a furanosyl sugar comprising a non-hydrogen substituent in place of at least one hydrogen of an unmodified sugar moiety. In certain embodiments, a modified furanosyl sugar moiety is a 2′-substituted sugar moiety. Such modified furanosyl sugar moieties include bicyclic sugars and non-bicyclic sugars. As used herein, “sugar surrogate” or means a modified sugar moiety having other than a furanosyl moiety that can link a nucleobase to another group, such as an internucleoside linkage, conjugate group, or terminal group in an oligonucleotide. Modified nucleosides comprising sugar surrogates can be incorporated into one or more positions within an oligonucleotide and such oligonucleotides are capable of hybridizing to complementary oligomeric compounds or nucleic acids.

As used herein, “Target nucleic acid” means a naturally occurring, identified nucleic acid. In certain embodiments, target nucleic acids are endogenous cellular nucleic acids, including, but not limited to RNA transcripts, pre-mRNA, mRNA, microRNA. In certain embodiments, target nucleic acids are viral nucleic acids. In certain embodiments, target nucleic acids are nucleic acids that an antisense compound is designed to affect.

As used herein, “Target region” means a portion of a target nucleic acid to which an antisense compound is designed to hybridize.

As used herein, “TCA motif” means three nucleosides having the nucleobase sequence TCA (5′-3′). Such nucleosides may have modified sugar moieties and/or modified internucleosides linkages. Unless otherwise indicated, the nucleosides of TCA motifs comprise unmodified 2′-deoxy sugar moieties and unmodified phosphodiester internucleoside linkages.

In certain embodiments, the oligomeric compounds as provided herein can be modified by covalent attachment of one or more terminal groups to the 5′ or 3′-terminal groups. A terminal group can also be attached at any other position at one of the terminal ends of the oligomeric compound. As used herein the terms “5′-terminal group”, “3′-terminal group”, “terminal group” and combinations thereof are meant to include useful groups known to the art skilled that can be placed on one or both of the terminal ends, including but not limited to the 5′ and 3′-ends of an oligomeric compound respectively, for various purposes such as enabling the tracking of the oligomeric compound (a fluorescent label or other reporter group), improving the pharmacokinetics or pharmacodynamics of the oligomeric compound (such as for example: uptake and/or delivery) or enhancing one or more other desirable properties of the oligomeric compound (a group for improving nuclease stability or binding affinity). In certain embodiments, 5′ and 3′-terminal groups include without limitation, modified or unmodified nucleosides; two or more linked nucleosides that are independently, modified or unmodified; conjugate groups; capping groups; phosphate moieties; and protecting groups.

I. Certain Oligonucleotides

In certain embodiments, the invention provides oligonucleotides, which consist of linked nucleosides. Oligonucleotides may be unmodified oligonucleotides (RNA or DNA) or may be modified oligonucleotides. Modified oligonucleotides comprise at least one modification relative to unmodified RNA or DNA (i.e., comprise at least one modified nucleoside (comprising a modified sugar moiety and/or a modified nucleobase) and/or at least one modified internucleoside linkage).

A. Certain Modified Nucleosides

Modified nucleosides comprise a modified sugar moiety or a modified nucleobase or both a modified sugar moiety and a modified nucleobase.

1. Certain Sugar Moieties

In certain embodiments, modified sugar moieties are non-bicyclic modified sugar moieties. In certain embodiments, modified sugar moieties are bicyclic or tricyclic sugar moieties. In certain embodiments, modified sugar moieties are sugar surrogates. Such sugar surrogates may comprise one or more substitutions corresponding to those of other types of modified sugar moieties.

In certain embodiments, modified sugar moieties are non-bicyclic modified sugar moieties comprising a furanosyl ring with one or more acyclic substituent, including but not limited to substituents at the 2′, 4′, and/or 5′ positions. In certain embodiments one or more acyclic substituent of non-bicyclic modified sugar moieties is branched. Examples of 2′-substituent groups suitable for non-bicyclic modified sugar moieties include but are not limited to: 2′-F, 2′-OCH₃ (“OMe” or “O-methyl”), and 2′-O(CH₂)₂OCH₃ (“MOE”). In certain embodiments, 2′-substituent groups are selected from among: halo, allyl, amino, azido, SH, CN, OCN, CF₃, OCF₃, O—C₁-C₁₀ alkoxy, O—C₁-C₁₀ substituted alkoxy, O—C₁-C₁₀ alkyl, O—C₁-C₁₀ substituted alkyl, S-alkyl, N(Rₘ)-alkyl, O-alkenyl, S-alkenyl, N(Rₘ)-alkenyl, O-alkynyl, S-alkynyl, N(Rₘ)-alkynyl, O-alkylenyl-O-alkyl, alkynyl, alkaryl, aralkyl, O-alkaryl, O-aralkyl, O(CH₂)₂SCH₃, O(CH₂)₂ON(Rₘ)(Rₙ) or OCH₂C(═O)—N(Rₘ)(Rₙ), where each Rₘ and Rₙ is, independently, H, an amino protecting group, or substituted or unsubstituted C₁-C₁₀ alkyl, and the 2′-substituent groups described in Cook et al., U.S. Pat. No. 6,531,584; Cook et al., U.S. Pat. No. 5,859,221; and Cook et al., U.S. Pat. No. 6,005,087. Certain embodiments of these 2′-substituent groups can be further substituted with one or more substituent groups independently selected from among: hydroxyl, amino, alkoxy, carboxy, benzyl, phenyl, nitro (NO₂), thiol, thioalkoxy, thioalkyl, halogen, alkyl, aryl, alkenyl and alkynyl. Examples of 4′-substituent groups suitable for non-bicyclic modified sugar moieties include but are not limited to alkoxy (e.g., methoxy), alkyl, and those described in Manoharan et al., WO 2015/106128. Examples of 5′-substituent groups suitable for non-bicyclic modified sugar moieties include but are not limited to: 5′-methyl (R or S), 5′-vinyl, and 5′-methoxy. In certain embodiments, non-bicyclic modified sugars comprise more than one non-bridging sugar substituent, for example, 2′-F-5′-methyl sugar moieties and the modified sugar moieties and modified nucleosides described in Migawa et al., WO 2008/101157 and Rajeev et al., US2013/0203836.).

In certain embodiments, a 2′-substituted nucleoside or 2′-non-bicyclic modified nucleoside comprises a sugar moiety comprising a non-bridging 2′-substituent group selected from: F, NH₂, N₃, OCF₃, OCH₃, O(CH₂)₃NH₂, CH₂CH═CH₂, OCH₂CH═CH₂, OCH₂CH₂OCH₃, O(CH₂)₂SCH₃, O(CH₂)₂ON(Rₘ)(Rₙ), O(CH₂)₂O(CH₂)₂N(CH₃)₂, and N-substituted acetamide (OCH₂C(═O)—N(Rₘ)(Rₙ)), where each Rₘ and Rₙ is, independently, H, an amino protecting group, or substituted or unsubstituted C₁-C₁₀ alkyl.

In certain embodiments, a 2′-substituted nucleoside or 2′-non-bicyclic modified nucleoside comprises a sugar moiety comprising a non-bridging 2′-substituent group selected from: F, OCF₃, OCH₃, OCH₂CH₂OCH₃, O(CH₂)₂SCH₃, O(CH₂)₂ON(CH₃)₂, O(CH₂)₂O(CH₂)₂N(CH₃)₂, and OCH₂C(═O)—N(H)CH₃ (“NMA”).

In certain embodiments, a 2′-substituted nucleoside or 2′-non-bicyclic modified nucleoside comprises a sugar moiety comprising a non-bridging 2′-substituent group selected from: F, OCH₃, and OCH₂CH₂OCH₃.

Nucleosides comprising modified sugar moieties, such as non-bicyclic modified sugar moieties, may be referred to by the position(s) of the substitution(s) on the sugar moiety of the nucleoside. For example, nucleosides comprising 2′-substituted or 2-modified sugar moieties are referred to as 2′-substituted nucleosides or 2-modified nucleosides.

Certain modified sugar moieties comprise a bridging sugar substituent that forms a second ring resulting in a bicyclic sugar moiety. In certain such embodiments, the bicyclic sugar moiety comprises a bridge between the 4′ and the 2′ furanose ring atoms. Examples of such 4′ to 2′ bridging sugar substituents include but are not limited to: 4′-CH₂-2′, 4′-(CH₂)₂-2′, 4′-(CH₂)₃-2′, 4′-CH₂—O-2′ (“LNA”), 4′-CH₂—S-2′, 4′-(CH₂)₂—O-2′ (“ENA”), 4′-CH(CH₃)—O-2′ (referred to as “constrained ethyl” or “cEt” when in the S configuration), 4′-CH₂—O—CH₂-2′, 4′-CH₂—N(R)-2′, 4′-CH(CH₂OCH₃)—O-2′ (“constrained MOE” or “cMOE”) and analogs thereof (see, e.g., Seth et al., U.S. Pat. No. 7,399,845, Bhat et al., U.S. Pat. No. 7,569,686, Swayze et al., U.S. Pat. No. 7,741,457, and Swayze et al., U.S. Pat. No. 8,022,193), 4′-C(CH₃)(CH₃)—O-2′ and analogs thereof (see, e.g., Seth et al., U.S. Pat. No. 8,278,283), 4′-CH₂—N(OCH₃)-2′ and analogs thereof (see, e.g., Prakash et al., U.S. Pat. No. 8,278,425), 4′-CH₂—O—N(CH₃)-2′ (see, e.g., Allerson et al., U.S. Pat. No. 7,696,345 and Allerson et al., U.S. Pat. No. 8,124,745), 4′-CH₂—C(H)(CH₃)-2′ (see, e.g., Zhou, et al., J. Org. Chem., 2009, 74, 118-134), 4′-CH₂—C(═CH₂)-2′ and analogs thereof (see e.g., Seth et al., U.S. Pat. No. 8,278,426), 4′-C(RₐR_{b})—N(R)—O-2′, 4′-C(RₐR_{b})—O—N(R)-2′, 4′-CH₂—O—N(R)-2′, and 4′-CH₂—N(R)—O-2′, wherein each R, Ra, and R_{b} is, independently, H, a protecting group, or C₁-C₁₂ alkyl (see, e.g. Imanishi et al., U.S. Pat. No. 7,427,672).

In certain embodiments, such 4′ to 2′ bridges independently comprise from 1 to 4 linked groups independently selected from: —[C(Rₐ)(R_{b})]ₙ—, —[C(Rₐ)(R_{b})]ₙ—O—, —C(Rₐ)═C(R_{b})—, —C(Rₐ)═N, —C(═NRₐ)—, —C(═O)—, —C(═S)—, —O—, —Si(Rₐ)₂—, —S(═O)ₓ—, and —N(Rₐ)—;

wherein:

x is 0, 1, or 2;

n is 1, 2, 3, or 4;

each Rₐ and R_{b} is, independently, H, a protecting group, hydroxyl, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, heterocycle radical, substituted heterocycle radical, heteroaryl, substituted heteroaryl, C₅-C₇ alicyclic radical, substituted C₅-C₇ alicyclic radical, halogen, OJ₁, NJ₁J₂, SJ₁, N₃, COOJ₁, acyl (C(═O)—H), substituted acyl, CN, sulfonyl (S(═O)₂-J₁), or sulfoxyl (S(═O)-J₁); and

each J₁ and J₂ is, independently, H, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, acyl (C(═O)—H), substituted acyl, a heterocycle radical, a substituted heterocycle radical, C₁-C₁₂ aminoalkyl, substituted C₁-C₁₂ aminoalkyl, or a protecting group.

Additional bicyclic sugar moieties are known in the art, see, for example: Freier et al., Nucleic Acids Research, 1997, 25(22), 4429-4443, Albaek et al., J. Org. Chem., 2006, 71, 7731-7740, Singh et al., Chem. Commun., 1998, 4, 455-456; Koshkin et al., Tetrahedron, 1998, 54, 3607-3630; Kumar et al., Bioorg. Med. Chem. Lett., 1998, 8, 2219-2222; Singh et al., J. Org. Chem., 1998, 63, 10035-10039; Srivastava et al., J. Am. Chem. Soc., 20017, 129, 8362-8379; Wengel et a., U.S. Pat. No. 7,053,207; Imanishi et al., U.S. Pat. No. 6,268,490; Imanishi et al., U.S. Pat. No. 6,770,748; Imanishi et al., U.S. RE44,779; Wengel et al., U.S. Pat. No. 6,794,499; Wengel et al., U.S. Pat. No. 6,670,461; Wengel et al., U.S. Pat. No. 7,034,133; Wengel et al., U.S. Pat. No. 8,080,644; Wengel et al., U.S. Pat. No. 8,034,909; Wengel et al., U.S. Pat. No. 8,153,365; Wengel et al., U.S. Pat. No. 7,572,582; and Ramasamy et al., U.S. Pat. No. 6,525,191; Torsten et al., WO 2004/106356; Wengel et al., WO 1999/014226; Seth et al., WO 2007/134181; Seth et al., U.S. 7,547,684; Seth et al., U.S. Pat. No. 7,666,854; Seth et al., U.S. Pat. No. 8,088,746; Seth et al., U.S. Pat. No. 7,750,131; Seth et al., U.S. Pat. No. 8,030,467; Seth et al., U.S. Pat. No. 8,268,980; Seth et al., U.S. Pat. No. 8,546,556; Seth et al., U.S. Pat. No. 8,530,640; Migawa et al., U.S. Pat. No. 9,012,421; Seth et al., U.S. Pat. No. 8,501,805; and U.S. Patent Publication Nos. Allerson et al., US2008/0039618 and Migawa et al., US2015/0191727.

In certain embodiments, bicyclic sugar moieties and nucleosides incorporating such bicyclic sugar moieties are further defined by isomeric configuration. For example, an LNA nucleoside (described herein) may be in the α-L configuration or in the β-D configuration.

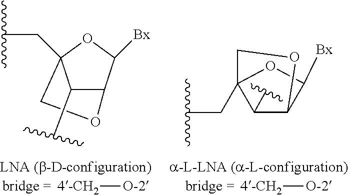

α-L-methyleneoxy (4′-CH₂—O-2′) or α-L-LNA bicyclic nucleosides have been incorporated into antisense oligonucleotides that showed antisense activity (Frieden et al., Nucleic Acids Research, 2003, 21, 6365-6372). Herein, general descriptions of bicyclic nucleosides include both isomeric configurations. When the positions of specific bicyclic nucleosides (e.g., LNA or cEt) are identified in exemplified embodiments herein, they are in the β-D configuration, unless otherwise specified.

In certain embodiments, modified sugar moieties comprise one or more non-bridging sugar substituent and one or more bridging sugar substituent (e.g., 5′-substituted and 4′-2′ bridged sugars).

In certain embodiments, modified sugar moieties are sugar surrogates. In certain such embodiments, the oxygen atom of the sugar moiety is replaced, e.g., with a sulfur, carbon or nitrogen atom. In certain such embodiments, such modified sugar moieties also comprise bridging and/or non-bridging substituents as described herein. For example, certain sugar surrogates comprise a 4′-sulfur atom and a substitution at the 2′-position (see, e.g., Bhat et al., U.S. Pat. No. 7,875,733 and Bhat et al., U.S. Pat. No. 7,939,677) and/or the 5′ position.

In certain embodiments, sugar surrogates comprise rings having other than 5 atoms. For example, in certain embodiments, a sugar surrogate comprises a six-membered tetrahydropyran (“THP”). Such tetrahydropyrans may be further modified or substituted. Nucleosides comprising such modified tetrahydropyrans include but are not limited to hexitol nucleic acid (“HNA”), anitol nucleic acid (“ANA”), manitol nucleic acid (“MNA”) (see, e.g., Leumann, C J. Bioorg. & Med. Chem. 2002, 10, 841-854), fluoro HNA:

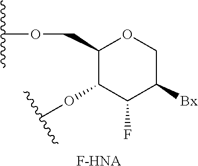

(“F-HNA”, see e.g. Swayze et al., U.S. Pat. No. 8,088,904; Swayze et al., U.S. Pat. No. 8,440,803; Swayze et al., U.S. Pat. No. 8,796,437; and Swayze et al., U.S. Pat. No. 9,005,906; F-HNA can also be referred to as a F-THP or 3′-fluoro tetrahydropyran), and nucleosides comprising additional modified THP compounds having the formula:

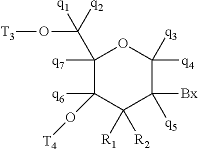

wherein, independently, for each of said modified THP nucleoside:

Bx is a nucleobase moiety;

T₃ and T₄ are each, independently, an internucleoside linking group linking the modified THP nucleoside to the remainder of an oligonucleotide or one of T₃ and T₄ is an internucleoside linking group linking the modified THP nucleoside to the remainder of an oligonucleotide and the other of T₃ and T₄ is H, a hydroxyl protecting group, a linked conjugate group, or a 5′ or 3′-terminal group; q₁, q₂, q₃, q₄, q₅, q₆ and q₇ are each, independently, H, C₁-C₆ alkyl, substituted C₁-C₆ alkyl, C₂-C₆ alkenyl, substituted C₂-C₆ alkenyl, C₂-C₆ alkynyl, or substituted C₂-C₆ alkynyl; and each of R₁ and R₂ is independently selected from among: hydrogen, halogen, substituted or unsubstituted alkoxy, N₁J₂, SJ₁, N₃, OC(═X)J₁, OC(═X)N₁J₂, NJ₃C(═X)N₁J₂, and CN, wherein X is O, S or NJ₁, and each J₁, J₂, and J₃ is, independently, H or C₁-C₆ alkyl.

In certain embodiments, modified THP nucleosides are provided wherein q₁, q₂, q₃, q₄, q₅, q₆ and q₇ are each H. In certain embodiments, at least one of q₁, q₂, q₃, q₄, q₅, q₆ and q₇ is other than H. In certain embodiments, at least one of q₁, q₂, q₃, q₄, q₅, q₆ and q₇ is methyl. In certain embodiments, modified THP nucleosides are provided wherein one of R₁ and R₂ is F. In certain embodiments, R₁ is F and R₂ is H, in certain embodiments, R₁ is methoxy and R₂ is H, and in certain embodiments, R₁ is methoxyethoxy and R₂ is H.

In certain embodiments, sugar surrogates comprise rings having more than 5 atoms and more than one heteroatom. For example, nucleosides comprising morpholino sugar moieties and their use in oligonucleotides have been reported (see, e.g., Braasch et al., Biochemistry, 2002, 41, 4503-4510 and Summerton et al., U.S. Pat. No. 5,698,685; Summerton et al., U.S. Pat. No. 5,166,315; Summerton et al., U.S. Pat. No. 5,185,444; and Summerton et al., U.S. Pat. No. 5,034,506). As used here, the term “morpholino” means a sugar surrogate having the following structure:

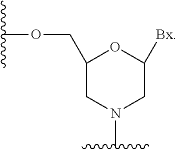

In certain embodiments, morpholinos may be modified, for example by adding or altering various substituent groups from the above morpholino structure. Such sugar surrogates are referred to herein as “modified morpholinos.”

In certain embodiments, sugar surrogates comprise acyclic moieties. Examples of nucleosides and oligonucleotides comprising such acyclic sugar surrogates include but are not limited to: peptide nucleic acid (“PNA”), acyclic butyl nucleic acid (see, e.g., Kumar et al., Org. Biomol. Chem., 2013, 11, 5853-5865), and nucleosides and oligonucleotides described in Manoharan et al., WO2011/133876.

Many other bicyclic and tricyclic sugar and sugar surrogate ring systems are known in the art that can be used in modified nucleosides).

1. Certain Modified Nucleobases

In certain embodiments, modified oligonucleotides comprise one or more nucleoside comprising an unmodified nucleobase. In certain embodiments, modified oligonucleotides comprise one or more nucleoside comprising a modified nucleobase. In certain embodiments, modified oligonucleotides comprise one or more nucleoside that does not comprise a nucleobase, referred to as an abasic nucleoside.

In certain embodiments, modified nucleobases are selected from: 5-substituted pyrimidines, 6-azapyrimidines, alkyl or alkynyl substituted pyrimidines, alkyl substituted purines, and N-2, N-6 and 0-6 substituted purines. In certain embodiments, modified nucleobases are selected from: 2-aminopropyladenine, 5-hydroxymethyl cytosine, xanthine, hypoxanthine, 2-aminoadenine, 6-N-methylguanine, 6-N-methyladenine, 2-propyladenine, 2-thiouracil, 2-thiothymine and 2-thiocytosine, 5-propynyl (—C≡C—CH₃) uracil, 5-propynylcytosine, 6-azouracil, 6-azocytosine, 6-azothymine, 5-ribosyluracil (pseudouracil), 4-thiouracil, 8-halo, 8-amino, 8-thiol, 8-thioalkyl, 8-hydroxyl, 8-aza and other 8-substituted purines, 5-halo, particularly 5-bromo, 5-trifluoromethyl, 5-halouracil, and 5-halocytosine, 7-methylguanine, 7-methyladenine, 2-F-adenine, 2-aminoadenine, 7-deazaguanine, 7-deazaadenine, 3-deazaguanine, 3-deazaadenine, 6-N-benzoyladenine, 2-N-isobutyrylguanine, 4-N-benzoylcytosine, 4-N-benzoyluracil, 5-methyl 4-N-benzoylcytosine, 5-methyl 4-N-benzoyluracil, universal bases, hydrophobic bases, promiscuous bases, size-expanded bases, and fluorinated bases. Further modified nucleobases include tricyclic pyrimidines, such as 1,3-diazaphenoxazine-2-one, 1,3-diazaphenothiazine-2-one and 9-(2-aminoethoxy)-1,3-diazaphenoxazine-2-one (G-clamp). Modified nucleobases may also include those in which the purine or pyrimidine base is replaced with other heterocycles, for example 7-deaza-adenine, 7-deazaguanosine, 2-aminopyridine and 2-pyridone. Further nucleobases include those disclosed in Merigan et al., U.S. Pat. No. 3,687,808, those disclosed in The Concise Encyclopedia Of Polymer Science And Engineering, Kroschwitz, J. I., Ed., John Wiley & Sons, 1990, 858-859; Englisch et al., Angewandte Chemie, International Edition, 1991, 30, 613; Sanghvi, Y. S., Chapter 15, Antisense Research and Applications, Crooke, S. T. and Lebleu, B., Eds., CRC Press, 1993, 273-288; and those disclosed in Chapters 6 and 15, Antisense Drug Technology, Crooke S. T., Ed., CRC Press, 2008, 163-166 and 442-443.

Publications that teach the preparation of certain of the above noted modified nucleobases as well as other modified nucleobases include without limitation, Manoharan et al., US2003/0158403; Manoharan et al., US2003/0175906; Dinh et al., U.S. Pat. No. 4,845,205; Spielvogel et al., U.S. Pat. No. 5,130,302; Rogers et al., U.S. Pat. No. 5,134,066; Bischofberger et al., U.S. Pat. No. 5,175,273; Urdea et al., U.S. Pat. No. 5,367,066; Benner et al., U.S. Pat. No. 5,432,272; Matteucci et al., U.S. Pat. No. 5,434,257; Gmeiner et al., U.S. Pat. No. 5,457,187; Cook et al., U.S. Pat. No. 5,459,255; Froehler et al., U.S. Pat. No. 5,484,908; Matteucci et al., U.S. Pat. No. 5,502,177; Hawkins et al., U.S. Pat. No. 5,525,711; Haralambidis et al., U.S. Pat. No. 5,552,540; Cook et al., U.S. Pat. No. 5,587,469; Froehler et al., U.S. Pat. No. 5,594,121; Switzer et al., U.S. Pat. No. 5,596,091; Cook et al., U.S. Pat. No. 5,614,617; Froehler et al., U.S. Pat. No. 5,645,985; Cook et al., U.S. Pat. No. 5,681,941; Cook et al., U.S. Pat. No. 5,811,534; Cook et al., U.S. Pat. No. 5,750,692; Cook et al., U.S. Pat. No. 5,948,903; Cook et al., U.S. Pat. No. 5,587,470; Cook et al., U.S. Pat. No. 5,457,191; Matteucci et al., U.S. Pat. No. 5,763,588; Froehler et al., U.S. Pat. No. 5,830,653; Cook et al., U.S. Pat. No. 5,808,027; Cook et al., 6,166,199; and Matteucci et al., U.S. Pat. No. 6,005,096.

B. Certain Modified Internucleoside Linkages

In certain embodiments, nucleosides of modified oligonucleotides may be linked together using any internucleoside linkage. The two main classes of internucleoside linking groups are defined by the presence or absence of a phosphorus atom. Representative phosphorus-containing internucleoside linkages include but are not limited to phosphates, which contain a phosphodiester bond (“P═O”) (also referred to as unmodified or naturally occurring linkages), phosphotriesters, methylphosphonates, phosphoramidates, and phosphorothioates (“P═S”), and phosphorodithioates (“HS-P=S”). Representative non-phosphorus containing internucleoside linking groups include but are not limited to methylenemethylimino (—CH₂—N(CH₃)—O—CH₂—), thiodiester, thionocarbamate (—O—C(═O)(NH)—S—); siloxane (—O—SiH₂—O—); and N,N′-dimethylhydrazine (—CH₂—N(CH₃)—N(CH₃)—). Modified internucleoside linkages, compared to naturally occurring phosphodiester linkages, can be used to alter, typically increase, nuclease resistance of the oligonucleotide. In certain embodiments, internucleoside linkages having a chiral atom can be prepared as a racemic mixture, or as separate enantiomers. Representative chiral internucleoside linkages include but are not limited to alkylphosphonates and phosphorothioates. Methods of preparation of phosphorous-containing and non-phosphorous-containing internucleoside linkages are well known to those skilled in the art.

Neutral internucleoside linkages include, without limitation, phosphotriesters, methylphosphonates, MMI (3′-CH₂—N(CH₃)—O-5′), amide-3 (3′-CH₂—C(═O)—N(H)-5′), amide-4 (3′-CH₂—N(H)—C(═O)-5′), formacetal (3′-O—CH₂—O-5′), methoxypropyl, and thioformacetal (3′-S—CH₂—O-5′). Further neutral internucleoside linkages include nonionic linkages comprising siloxane (dialkylsiloxane), carboxylate ester, carboxamide, sulfide, sulfonate ester and amides (See for example: Carbohydrate Modifications in Antisense Research; Y. S. Sanghvi and P. D. Cook, Eds., ACS Symposium Series 580; Chapters 3 and 4, 40-65). Further neutral internucleoside linkages include nonionic linkages comprising mixed N, O, S and CH₂ component parts.

C. Certain Motifs

In certain embodiments, modified oligonucleotides comprise one or more modified nucleoside comprising a modified sugar. In certain embodiments, modified oligonucleotides comprise one or more modified nucleosides comprising a modified nucleobase. In certain embodiments, modified oligonucleotides comprise one or more modified internucleoside linkage. In such embodiments, the modified, unmodified, and differently modified sugar moieties, nucleobases, and/or internucleoside linkages of a modified oligonucleotide define a pattern or motif. In certain embodiments, the patterns of sugar moieties, nucleobases, and internucleoside linkages are each independent of one another. Thus, a modified oligonucleotide may be described by its sugar motif, nucleobase motif and/or internucleoside linkage motif (as used herein, nucleobase motif describes the modifications to the nucleobases independent of the sequence of nucleobases).

1. Certain Sugar Motifs

In certain embodiments, oligonucleotides comprise one or more type of modified sugar and/or unmodified sugar moiety arranged along the oligonucleotide or region thereof in a defined pattern or sugar motif. In certain instances, such sugar motifs include but are not limited to any of the sugar modifications discussed herein.

In certain embodiments, modified oligonucleotides comprise or consist of a region having a gapmer motif, which comprises two external regions or “wings” and a central or internal region or “gap.” The three regions of a gapmer motif (the 5′-wing, the gap, and the 3′-wing) form a contiguous sequence of nucleosides wherein at least some of the sugar moieties of the nucleosides of each of the wings differ from at least some of the sugar moieties of the nucleosides of the gap. Specifically, at least the sugar moieties of the nucleosides of each wing that are closest to the gap (the 3′-most nucleoside of the 5′-wing and the 5′-most nucleoside of the 3′-wing) differ from the sugar moiety of the neighboring gap nucleosides, thus defining the boundary between the wings and the gap (i.e., the wing/gap junction). In certain embodiments, the sugar moieties within the gap are the same as one another. In certain embodiments, the gap includes one or more nucleoside having a sugar moiety that differs from the sugar moiety of one or more other nucleosides of the gap. In certain embodiments, the sugar motifs of the two wings are the same as one another (symmetric gapmer). In certain embodiments, the sugar motif of the 5′-wing differs from the sugar motif of the 3′-wing (asymmetric gapmer).

In certain embodiments, the wings of a gapmer comprise 1-5 nucleosides. In certain embodiments, the wings of a gapmer comprise 2-5 nucleosides. In certain embodiments, the wings of a gapmer comprise 3-5 nucleosides. In certain embodiments, the nucleosides of a gapmer are all modified nucleosides.

In certain embodiments, the gap of a gapmer comprises 7-12 nucleosides. In certain embodiments, the gap of a gapmer comprises 7-10 nucleosides. In certain embodiments, the gap of a gapmer comprises 8-10 nucleosides. In certain embodiments, the gap of a gapmer comprises 10 nucleosides. In certain embodiment, each nucleoside of the gap of a gapmer is an unmodified 2′-deoxy nucleoside.

In certain embodiments, the gapmer is a deoxy gapmer. In such embodiments, the nucleosides on the gap side of each wing/gap junction are unmodified 2′-deoxy nucleosides and the nucleosides on the wing sides of each wing/gap junction are modified nucleosides. In certain such embodiments, each nucleoside of the gap is an unmodified 2′-deoxy nucleoside. In certain such embodiments, each nucleoside of each wing is a modified nucleoside.

In certain embodiments, modified oligonucleotides comprise or consist of a region having a fully modified sugar motif. In such embodiments, each nucleoside of the fully modified region of the modified oligonucleotide comprises a modified sugar moiety. In certain such embodiments, each nucleoside to the entire modified oligonucleotide comprises a modified sugar moiety. In certain embodiments, modified oligonucleotides comprise or consist of a region having a fully modified sugar motif, wherein each nucleoside within the fully modified region comprises the same modified sugar moiety, referred to herein as a uniformly modified sugar motif. In certain embodiments, a fully modified oligonucleotide is a uniformly modified oligonucleotide. In certain embodiments, each nucleoside of a uniformly modified oligonucleotide comprises the same 2′-modification.

2. Certain Nucleobase Motifs

In certain embodiments, oligonucleotides comprise modified and/or unmodified nucleobases arranged along the oligonucleotide or region thereof in a defined pattern or motif. In certain embodiments, each nucleobase is modified. In certain embodiments, none of the nucleobases are modified. In certain embodiments, each purine or each pyrimidine is modified. In certain embodiments, each adenine is modified. In certain embodiments, each guanine is modified. In certain embodiments, each thymine is modified. In certain embodiments, each uracil is modified. In certain embodiments, each cytosine is modified. In certain embodiments, some or all of the cytosine nucleobases in a modified oligonucleotide are 5-methylcytosines.

In certain embodiments, modified oligonucleotides comprise a block of modified nucleobases. In certain such embodiments, the block is at the 3′-end of the oligonucleotide. In certain embodiments the block is within 3 nucleosides of the 3′-end of the oligonucleotide. In certain embodiments, the block is at the 5′-end of the oligonucleotide. In certain embodiments the block is within 3 nucleosides of the 5′-end of the oligonucleotide.

In certain embodiments, oligonucleotides having a gapmer motif comprise a nucleoside comprising a modified nucleobase. In certain such embodiments, one nucleoside comprising a modified nucleobase is in the central gap of an oligonucleotide having a gapmer motif. In certain such embodiments, the sugar moiety of said nucleoside is a 2′-deoxyribosyl moiety. In certain embodiments, the modified nucleobase is selected from: a 2-thiopyrimidine and a 5-propynepyrimidine.

3. Certain Internucleoside Linkage Motifs

In certain embodiments, oligonucleotides comprise modified and/or unmodified internucleoside linkages arranged along the oligonucleotide or region thereof in a defined pattern or motif. In certain embodiments, essentially each internucleoside linking group is a phosphate internucleoside linkage (P═O). In certain embodiments, each internucleoside linking group of a modified oligonucleotide is a phosphorothioate (P═S). In certain embodiments, each internucleoside linking group of a modified oligonucleotide is independently selected from a phosphorothioate and phosphate internucleoside linkage. In certain embodiments, the sugar motif of a modified oligonucleotide is a gapmer and the internucleoside linkages within the gap are all modified. In certain such embodiments, some or all of the internucleoside linkages in the wings are phosphodiester linkages. In certain embodiments, the terminal internucleoside linkages are modified.

D. Certain Lengths

In certain embodiments, oligonucleotides (including modified oligonucleotides) can have any of a variety of ranges of lengths. In certain embodiments, oligonucleotides consist of X to Y linked nucleosides, where X represents the fewest number of nucleosides in the range and Y represents the largest number nucleosides in the range. In certain such embodiments, X and Y are each independently selected from 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50; provided that X≤Y. For example, in certain embodiments, oligonucleotides consist of 12 to 13, 12 to 14, 12 to 15, 12 to 16, 12 to 17, 12 to 18, 12 to 19, 12 to 20, 12 to 21, 12 to 22, 12 to 23, 12 to 24, 12 to 25, 12 to 26, 12 to 27, 12 to 28, 12 to 29, 12 to 30, 13 to 14, 13 to 15, 13 to 16, 13 to 17, 13 to 18, 13 to 19, 13 to 20, 13 to 21, 13 to 22, 13 to 23, 13 to 24, 13 to 25, 13 to 26, 13 to 27, 13 to 28, 13 to 29, 13 to 30, 14 to 15, 14 to 16, 14 to 17, 14 to 18, 14 to 19, 14 to 20, 14 to 21, 14 to 22, 14 to 23, 14 to 24, 14 to 25, 14 to 26, 14 to 27, 14 to 28, 14 to 29, 14 to 30, 15 to 16, 15 to 17, 15 to 18, 15 to 19, 15 to 20, 15 to 21, 15 to 22, 15 to 23, 15 to 24, 15 to 25, 15 to 26, 15 to 27, 15 to 28, 15 to 29, 15 to 30, 16 to 17, 16 to 18, 16 to 19, 16 to 20, 16 to 21, 16 to 22, 16 to 23, 16 to 24, 16 to 25, 16 to 26, 16 to 27, 16 to 28, 16 to 29, 16 to 30, 17 to 18, 17 to 19, 17 to 20, 17 to 21, 17 to 22, 17 to 23, 17 to 24, 17 to 25, 17 to 26, 17 to 27, 17 to 28, 17 to 29, 17 to 30, 18 to 19, 18 to 20, 18 to 21, 18 to 22, 18 to 23, 18 to 24, 18 to 25, 18 to 26, 18 to 27, 18 to 28, 18 to 29, 18 to 30, 19 to 20, 19 to 21, 19 to 22, 19 to 23, 19 to 24, 19 to 25, 19 to 26, 19 to 29, 19 to 28, 19 to 29, 19 to 30, 20 to 21, 20 to 22, 20 to 23, 20 to 24, 20 to 25, 20 to 26, 20 to 27, 20 to 28, 20 to 29, 20 to 30, 21 to 22, 21 to 23, 21 to 24, 21 to 25, 21 to 26, 21 to 27, 21 to 28, 21 to 29, 21 to 30, 22 to 23, 22 to 24, 22 to 25, 22 to 26, 22 to 27, 22 to 28, 22 to 29, 22 to 30, 23 to 24, 23 to 25, 23 to 26, 23 to 27, 23 to 28, 23 to 29, 23 to 30, 24 to 25, 24 to 26, 24 to 27, 24 to 28, 24 to 29, 24 to 30, 25 to 26, 25 to 27, 25 to 28, 25 to 29, 25 to 30, 26 to 27, 26 to 28, 26 to 29, 26 to 30, 27 to 28, 27 to 29, 27 to 30, 28 to 29, 28 to 30, or 29 to 30 linked nucleosides

E. Certain Modified Oligonucleotides

In certain embodiments, the above modifications (sugar, nucleobase, internucleoside linkage) are incorporated into a modified oligonucleotide. In certain embodiments, modified oligonucleotides are characterized by their modification motifs and overall lengths. In certain embodiments, such parameters are each independent of one another. Thus, unless otherwise indicated, each internucleoside linkage of an oligonucleotide having a gapmer sugar motif may be modified or unmodified and may or may not follow the gapmer modification pattern of the sugar modifications. For example, the internucleoside linkages within the wing regions of a sugar gapmer may be the same or different from one another and may be the same or different from the internucleoside linkages of the gap region of the sugar motif. Likewise, such sugar gapmer oligonucleotides may comprise one or more modified nucleobase independent of the gapmer pattern of the sugar modifications. Furthermore, in certain instances, an oligonucleotide is described by an overall length or range and by lengths or length ranges of two or more regions (e.g., a regions of nucleosides having specified sugar modifications), in such circumstances it may be possible to select numbers for each range that result in an oligonucleotide having an overall length falling outside the specified range. In such circumstances, both elements must be satisfied. For example, in certain embodiments, a modified oligonucleotide consists if of 15-20 linked nucleosides and has a sugar motif consisting of three regions, A, B, and C, wherein region A consists of 2-6 linked nucleosides having a specified sugar motif, region B consists of 6-10 linked nucleosides having a specified sugar motif, and region C consists of 2-6 linked nucleosides having a specified sugar motif. Such embodiments do not include modified oligonucleotides where A and C each consist of 6 linked nucleosides and B consists of 10 linked nucleosides (even though those numbers of nucleosides are permitted within the requirements for A, B, and C) because the overall length of such oligonucleotide is 22, which exceeds the upper limit of the overall length of the modified oligonucleotide (20). Herein, if a description of an oligonucleotide is silent with respect to one or more parameter, such parameter is not limited. Thus, a modified oligonucleotide described only as having a gapmer sugar motif without further description may have any length, internucleoside linkage motif, and nucleobase motif. Unless otherwise indicated, all modifications are independent of nucleobase sequence.

F. Nucleobase Sequence

In certain embodiments, oligonucleotides (unmodified or modified oligonucleotides) are further described by their nucleobase sequence. In certain embodiments oligonucleotides have a nucleobase sequence that is complementary to a second oligonucleotide or an identified reference nucleic acid, such as a target nucleic acid. In certain such embodiments, a region of an oligonucleotide has a nucleobase sequence that is complementary to a second oligonucleotide or an identified reference nucleic acid, such as a target nucleic acid. In certain embodiments, the nucleobase sequence of a region or entire length of an oligonucleotide is at least 50%, at least 60%, at least 70%, at least 80%, at least 90%, at least 95%, or 100% complementary to the second oligonucleotide or nucleic acid, such as a target nucleic acid.

II. Certain Oligomeric Compounds

In certain embodiments, the invention provides oligomeric compounds, which consist of an oligonucleotide (modified or unmodified) and optionally one or more terminal groups such as a conjugate group. Conjugate groups consist of one or more conjugate group and a conjugate linking group which links the conjugate group to the oligonucleotide. Conjugate groups may be attached to either or both ends of an oligonucleotide and/or at any internal position. In certain embodiments, conjugate groups are attached to the 2′-position of a nucleoside of a modified oligonucleotide. In certain embodiments, conjugate groups that are attached to either or both ends of an oligonucleotide are terminal groups. In certain such embodiments, conjugate groups or terminal groups are attached at the 3′ and/or 5′-end of oligonucleotides. In certain such embodiments, conjugate groups (or other terminal groups) are attached at the 3′-end of oligonucleotides. In certain embodiments, conjugate groups are attached near the 3′-end of oligonucleotides. In certain embodiments, conjugate groups (or terminal groups) are attached at the 5′-end of oligonucleotides. In certain embodiments, conjugate groups are attached near the 5′-end of oligonucleotides.

Examples of terminal groups include but are not limited to conjugate groups, capping groups, phosphate moieties, protecting groups, abasic nucleosides, modified or unmodified nucleosides, and two or more nucleosides that are independently modified or unmodified.

A. Certain Conjugate Groups

In certain embodiments, oligonucleotides are covalently attached to one or more conjugate groups. In certain embodiments, conjugate groups modify one or more properties of the attached oligonucleotide, including but not limited to pharmacodynamics, pharmacokinetics, stability, binding, absorption, tissue distribution, cellular distribution, cellular uptake, charge and clearance. In certain embodiments, conjugate groups impart a new property on the attached oligonucleotide, e.g., fluorophores or reporter groups that enable detection of the oligonucleotide. Certain conjugate groups and conjugate moieties have been described previously, for example: cholesterol moiety (Letsinger et al., Proc. Natl. Acad. Sci. USA, 1989, 86, 6553-6556), cholic acid (Manoharan et al., Bioorg. Med. Chem. Lett., 1994, 4, 1053-1060), a thioether, e.g., hexyl-S-tritylthiol (Manoharan et al., Ann. N.Y. Acad. Sci., 1992, 660, 306-309; Manoharan et al., Bioorg. Med. Chem. Lett., 1993, 3, 2765-2770), a thiocholesterol (Oberhauser et al., Nucl. Acids Res., 1992, 20, 533-538), an aliphatic chain, e.g., do-decan-diol or undecyl residues (Saison-Behmoaras et al., EMBO 1, 1991, 10, 1111-1118; Kabanov et al., FEBS Lett., 1990, 259, 327-330; Svinarchuk et al., Biochimie, 1993, 75, 49-54), a phospholipid, e.g., di-hexadecyl-rac-glycerol or triethyl-ammonium 1,2-di-O-hexadecyl-rac-glycero-3-H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Manoharan et al., Nucleosides & Nucleotides, 1995, 14, 969-973), or adamantane acetic acid a palmityl moiety (Mishra et al., Biochim. Biophys. Acta, 1995, 1264, 229-237), an octadecylamine or hexylamino-carbonyl-oxycholesterol moiety (Crooke et al., J. Pharmacol. Exp. Ther., 1996, 277, 923-937), a tocopherol group (Nishina et al., Molecular Therapy Nucleic Acids, 2015, 4, e220; and Nishina et al., Molecular Therapy, 2008, 16, 734-740), or a GalNAc cluster (e.g., WO2014/179620).

1. Conjugate Moieties

Conjugate moieties include, without limitation, intercalators, reporter molecules, polyamines, polyamides, peptides, carbohydrates (e.g., GalNAc), vitamin moieties, polyethylene glycols, thioethers, polyethers, cholesterols, thiocholesterols, cholic acid moieties, folate, lipids, phospholipids, biotin, phenazine, phenanthridine, anthraquinone, adamantane, acridine, fluoresceins, rhodamines, coumarins, fluorophores, and dyes.

In certain embodiments, a conjugate moiety comprises an active drug substance, for example, aspirin, warfarin, phenylbutazone, ibuprofen, suprofen, fen-bufen, ketoprofen, (S)-(+)-pranoprofen, carprofen, dansylsarcosine, 2,3,5-triiodobenzoic acid, fingolimod, flufenamic acid, folinic acid, a benzothiadiazide, chlorothiazide, a diazepine, indo-methicin, a barbiturate, a cephalosporin, a sulfa drug, an antidiabetic, an antibacterial or an antibiotic.

2. Conjugate Linkers

Conjugate moieties are attached to oligonucleotides through conjugate linkers. In certain oligomeric compounds, the conjugate linker is a single chemical bond (i.e., the conjugate moiety is attached directly to an oligonucleotide through a single bond). In certain oligomeric compounds, a conjugate moiety is attached to an oligonucleotide via a more complex conjugate linker comprising one or more conjugate linker moieties, which are sub-units making up a conjugate linker. In certain embodiments, the conjugate linker comprises a chain structure, such as a hydrocarbyl chain, or an oligomer of repeating units such as ethylene glycol, nucleosides, or amino acid units.

In certain embodiments, a conjugate linker comprises one or more groups selected from alkyl, amino, oxo, amide, disulfide, polyethylene glycol, ether, thioether, and hydroxylamino. In certain such embodiments, the conjugate linker comprises groups selected from alkyl, amino, oxo, amide and ether groups. In certain embodiments, the conjugate linker comprises groups selected from alkyl and amide groups. In certain embodiments, the conjugate linker comprises groups selected from alkyl and ether groups. In certain embodiments, the conjugate linker comprises at least one phosphorus moiety. In certain embodiments, the conjugate linker comprises at least one phosphate group. In certain embodiments, the conjugate linker includes at least one neutral linking group.

In certain embodiments, conjugate linkers, including the conjugate linkers described above, are bifunctional linking moieties, e.g., those known in the art to be useful for attaching conjugate groups to parent compounds, such as the oligonucleotides provided herein. In general, a bifunctional linking moiety comprises at least two functional groups. One of the functional groups is selected to bind to a particular site on a parent compound and the other is selected to bind to a conjugate group. Examples of functional groups used in a bifunctional linking moiety include but are not limited to electrophiles for reacting with nucleophilic groups and nucleophiles for reacting with electrophilic groups. In certain embodiments, bifunctional linking moieties comprise one or more groups selected from amino, hydroxyl, carboxylic acid, thiol, alkyl, alkenyl, and alkynyl.

Examples of conjugate linkers include but are not limited to pyrrolidine, 8-amino-3,6-dioxaoctanoic acid (ADO), succinimidyl 4-(N-maleimidomethyl) cyclohexane-1-carboxylate (SMCC) and 6-aminohexanoic acid (AHEX or AHA). Other conjugate linkers include but are not limited to substituted or unsubstituted C₁-C₁₀ alkyl, substituted or unsubstituted C₂-C₁₀ alkenyl or substituted or unsubstituted C₂-C₁₀ alkynyl, wherein a nonlimiting list of preferred substituent groups includes hydroxyl, amino, alkoxy, carboxy, benzyl, phenyl, nitro, thiol, thioalkoxy, halogen, alkyl, aryl, alkenyl and alkynyl.

In certain embodiments, conjugate linkers comprise 1-10 linker-nucleosides. In certain embodiments, such linker-nucleosides are modified nucleosides. In certain embodiments such linker-nucleosides comprise a modified sugar moiety. In certain embodiments, linker-nucleosides are unmodified. In certain embodiments, linker-nucleosides comprise an optionally protected heterocyclic base selected from a purine, substituted purine, pyrimidine or substituted pyrimidine. In certain embodiments, a cleavable moiety is a nucleoside selected from uracil, thymine, cytosine, 4-N-benzoylcytosine, 5-methylcytosine, 4-N-benzoyl-5-methylcytosine, adenine, 6-N-benzoyladenine, guanine and 2-N-isobutyrylguanine. In certain embodiments, a cleavable moiety is an unprotected β-D-2′-deoxyribonucleoside nucleoside selected from uracil, thymine, cytosine, adenine and guanine. It is typically desirable for linker-nucleosides to be cleaved from the oligomeric compound after it reaches a target tissue. Accordingly, linker-nucleosides are typically linked to one another and to the remainder of the oligomeric compound through cleavable bonds. In certain embodiments, such cleavable bonds are phosphodiester bonds. In certain embodiments, linker nucleosides are located at the 5′-terminus of the oligomeric compound. In certain embodiments, linker nucleosides are located at the 3′-terminus of the oligomeric compound.

Herein, linker-nucleosides are not considered to be part of the oligonucleotide. Accordingly, in embodiments in which an oligomeric compound comprises an oligonucleotide consisting of a specified number or range of linked nucleosides and/or a specified percent complementarity to a reference nucleic acid and the oligomeric compound also comprises a conjugate group comprising a conjugate linker comprising linker-nucleosides, those linker-nucleosides are not counted toward the length of the oligonucleotide and are not used in determining the percent complementarity of the oligonucleotide for the reference nucleic acid. For example, an oligomeric compound may comprise (1) a modified oligonucleotide consisting of 8-30 nucleosides and (2) a conjugate group comprising 1-10 linker-nucleosides that are contiguous with the nucleosides of the modified oligonucleotide. The total number of contiguous linked nucleosides in such an oligomeric compound is more than 30. Alternatively, an oligomeric compound may comprise a modified oligonucleotide consisting of 8-30 nucleosides and no conjugate group. The total number of contiguous linked nucleosides in such an oligomeric compound is no more than 30. Unless otherwise indicated conjugate linkers comprise no more than 10 linker-nucleosides. In certain embodiments, conjugate linkers comprise no more than 5 linker-nucleosides. In certain embodiments, conjugate linkers comprise no more than 3 linker-nucleosides. In certain embodiments, conjugate linkers comprise no more than 2 linker-nucleosides. In certain embodiments, conjugate linkers comprise no more than 1 linker-nucleoside.

In certain embodiments, it is desirable for a conjugate group to be cleaved from the oligonucleotide. For example, in certain circumstances oligomeric compounds comprising a particular conjugate moiety are better taken up by a particular cell type, but once the oligomeric compound has been taken up, it is desirable that the conjugate group be cleaved to release the unconjugated or parent oligonucleotide. Thus, certain conjugate linkers may comprise one or more cleavable moieties. In certain embodiments, a cleavable moiety is a cleavable bond. In certain embodiments, a cleavable moiety is a group of atoms comprising at least one cleavable bond. In certain embodiments, a cleavable moiety comprises a group of atoms having one, two, three, four, or more than four cleavable bonds. In certain embodiments, a cleavable moiety is selectively cleaved inside a cell or subcellular compartment, such as a lysosome. In certain embodiments, a cleavable moiety is selectively cleaved by endogenous enzymes, such as nucleases.

In certain embodiments, a cleavable bond is selected from among: an amide, an ester, an ether, one or both esters of a phosphodiester —O—P(═O)(—OH)O—, a phosphate ester —O—P(═O)(—OH)₂, a carbamate, disulfide or a linkage comprising any phosphorus moiety such as —P(═O)(—OH)—. In certain embodiments, a cleavable bond is one or both of the esters of a phosphodiester. In certain embodiments, a cleavable moiety comprises a phosphate or phosphodiester. In certain embodiments, the cleavable moiety is a phosphodiester linkage between an oligonucleotide and a conjugate moiety or conjugate group. In certain embodiments, the cleavable moiety is a phosphodiester linkage between an oligonucleotide and a conjugate linker attaching a conjugate group. In certain embodiments, the cleavable moiety is a phosphodiester linkage between an oligonucleotide and a conjugate group.

In certain embodiments, a cleavable moiety comprises or consists of one or more linker-nucleosides. In certain such embodiments, the one or more linker-nucleosides are linked to one another and/or to the remainder of the oligomeric compound through cleavable bonds. In certain embodiments, such cleavable bonds are unmodified phosphodiester bonds. In certain embodiments, a cleavable moiety is 2′-deoxy nucleoside that is attached to either the 3′ or 5′-terminal nucleoside of an oligonucleotide by a phosphate internucleoside linkage and covalently attached to the remainder of the conjugate linker or conjugate moiety by a phosphate or phosphorothioate linkage. In certain such embodiments, the cleavable moiety is 2′-deoxyadenosine. In certain embodiments, the cleavable moiety is from one to three nucleosides selected from 2′-deoxyadenosine 2′-deoxythymidine and 2′-deoxycytidine.

In certain embodiments, the conjugate group comprises a conjugate linker including a cleavable moiety having one of the formulas:

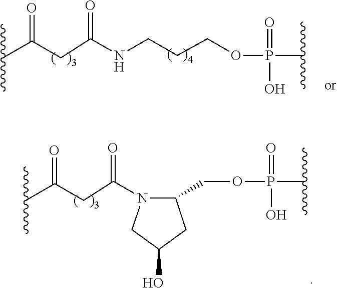

Wherein the phosphate group attaches the conjugate group to the gapped oligomeric compound. In certain embodiments, the phosphate group attaches the conjugate group to the 5′-terminal oxygen atom of the oligomeric compound. In certain embodiments, the phosphate group attaches the conjugate group to the 3′-terminal oxygen atom of the oligomeric compound.

3. Certain Cell-Targeting Conjugate Moieties

In certain embodiments, a conjugate group comprises a cell-targeting conjugate moiety. In certain embodiments, a conjugate group has the general formula:

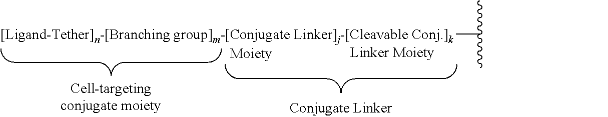

wherein n is from 1 to about 3, m is 0 when n is 1, m is 1 when n is 2 or greater, j is 1 or 0, and k is 1 or 0.

In certain embodiments, n is 1, j is 1 and k is 0. In certain embodiments, n is 1, j is 0 and k is 1. In certain embodiments, n is 1, j is 1 and k is 1. In certain embodiments, n is 2, j is 1 and k is 0. In certain embodiments, n is 2, j is 0 and k is 1. In certain embodiments, n is 2, j is 1 and k is 1. In certain embodiments, n is 3, j is 1 and k is 0. In certain embodiments, n is 3, j is 0 and k is 1. In certain embodiments, n is 3, j is 1 and k is 1.

In certain embodiments, conjugate groups comprise cell-targeting moieties that have at least one tethered ligand. In certain embodiments, cell-targeting moieties comprise two tethered ligands covalently attached to a branching group. In certain embodiments, cell-targeting moieties comprise three tethered ligands covalently attached to a branching group.

In certain embodiments, the cell-targeting moiety comprises a branching group comprising one or more groups selected from alkyl, amino, oxo, amide, disulfide, polyethylene glycol, ether, thioether and hydroxylamino groups. In certain embodiments, the branching group comprises a branched aliphatic group comprising groups selected from alkyl, amino, oxo, amide, disulfide, polyethylene glycol, ether, thioether and hydroxylamino groups. In certain such embodiments, the branched aliphatic group comprises groups selected from alkyl, amino, oxo, amide and ether groups. In certain such embodiments, the branched aliphatic group comprises groups selected from alkyl, amino and ether groups. In certain such embodiments, the branched aliphatic group comprises groups selected from alkyl and ether groups. In certain embodiments, the branching group comprises a mono or polycyclic ring system.

In certain embodiments, each tether of a cell-targeting moiety comprises one or more groups selected from alkyl, substituted alkyl, ether, thioether, disulfide, amino, oxo, amide, phosphodiester, and polyethylene glycol, in any combination. In certain embodiments, each tether is a linear aliphatic group comprising one or more groups selected from alkyl, ether, thioether, disulfide, amino, oxo, amide, and polyethylene glycol, in any combination. In certain embodiments, each tether is a linear aliphatic group comprising one or more groups selected from alkyl, phosphodiester, ether, amino, oxo, and amide, in any combination. In certain embodiments, each tether is a linear aliphatic group comprising one or more groups selected from alkyl, ether, amino, oxo, and amide, in any combination. In certain embodiments, each tether is a linear aliphatic group comprising one or more groups selected from alkyl, amino, and oxo, in any combination. In certain embodiments, each tether is a linear aliphatic group comprising one or more groups selected from alkyl, amide and oxo, in any combination. In certain embodiments, each tether is a linear aliphatic group comprising one or more groups selected from alkyl and amide, in any combination. In certain embodiments, each tether is a linear aliphatic group comprising one or more groups selected from alkyl and oxo, in any combination. In certain embodiments, each tether is a linear aliphatic group comprising one or more groups selected from alkyl and phosphodiester, in any combination. In certain embodiments, each tether comprises at least one phosphorus linking group or neutral linking group. In certain embodiments, each tether comprises a chain from about 6 to about 20 atoms in length. In certain embodiments, each tether comprises a chain from about 10 to about 18 atoms in length. In certain embodiments, each tether comprises about 10 atoms in chain length.

In certain embodiments, each ligand of a cell-targeting moiety has an affinity for at least one type of receptor on a target cell. In certain embodiments, each ligand has an affinity for at least one type of receptor on the surface of a mammalian liver cell. In certain embodiments, each ligand has an affinity for the hepatic asialoglycoprotein receptor (ASGP-R). In certain embodiments, each ligand is a carbohydrate. In certain embodiments, each ligand is, independently selected from galactose, N-acetyl galactoseamine (GalNAc), mannose, glucose, glucoseamine and fucose. In certain embodiments, each ligand is N-acetyl galactoseamine (GalNAc). In certain embodiments, the cell-targeting moiety comprises 3 GalNAc ligands. In certain embodiments, the cell-targeting moiety comprises 2 GalNAc ligands. In certain embodiments, the cell-targeting moiety comprises 1 GalNAc ligand.

In certain embodiments, each ligand of a cell-targeting moiety is a carbohydrate, carbohydrate derivative, modified carbohydrate, polysaccharide, modified polysaccharide, or polysaccharide derivative. In certain such embodiments, the conjugate group comprises a carbohydrate cluster (see, e.g., Maier et al., “Synthesis of Antisense Oligonucleotides Conjugated to a Multivalent Carbohydrate Cluster for Cellular Targeting,” Bioconjugate Chemistry, 2003, 14, 18-29 or Rensen et al., “Design and Synthesis of Novel N-Acetylgalactosamine-Terminated Glycolipids for Targeting of Lipoproteins to the Hepatic Asiaglycoprotein Receptor,” J. Med. Chem. 2004, 47, 5798-5808). In certain such embodiments, each ligand is an amino sugar or a thio sugar. For example, amino sugars may be selected from any number of compounds known in the art, such as sialic acid, α-D-galactosamine, β-muramic acid, 2-deoxy-2-methylamino-L-glucopyranose, 4,6-dideoxy-4-formamido-2,3-di-O-methyl-D-mannopyranose, 2-deoxy-2-sulfoamino-D-glucopyranose and N-sulfo-D-glucosamine, and N-glycoloyl-α-neuraminic acid. For example, thio sugars may be selected from 5-Thio-β-D-glucopyranose, methyl 2,3,4-tri-O-acetyl-1-thio-6-O-trityl-α-D-glucopyranoside, 4-thio-β-D-galactopyranose, and ethyl 3,4,6,7-tetra-O-acetyl-2-deoxy-1,5-dithio-α-D-gluco-heptopyranoside.

In certain embodiments, conjugate groups comprise a cell-targeting moiety having the formula:

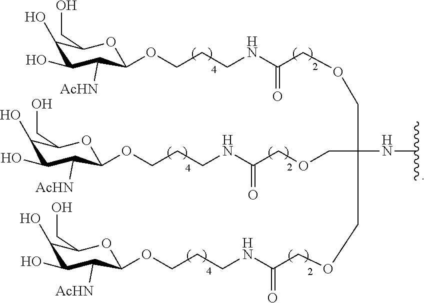

In certain embodiments, conjugate groups comprise a cell-targeting moiety having the formula:

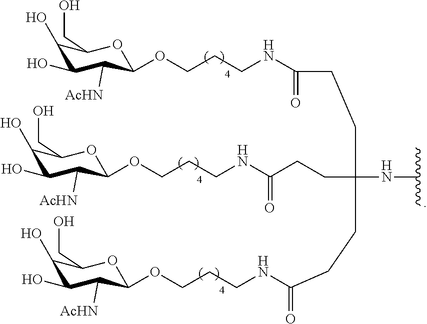

In certain embodiments, conjugate groups comprise a cell-targeting moiety having the formula:

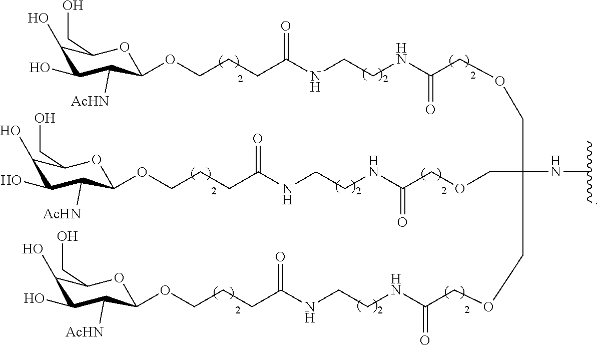

In certain embodiments, the conjugate group is attached to the 5′position of the oligomeric compound having the formula:

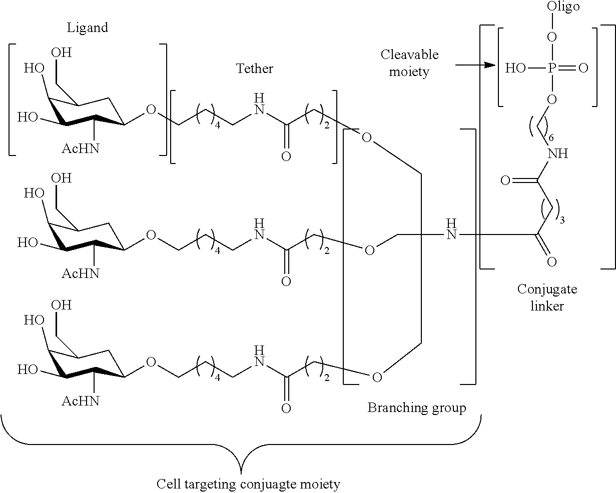

In certain embodiments, the conjugate group is attached to the 3′position of the oligomeric compound having the formula:

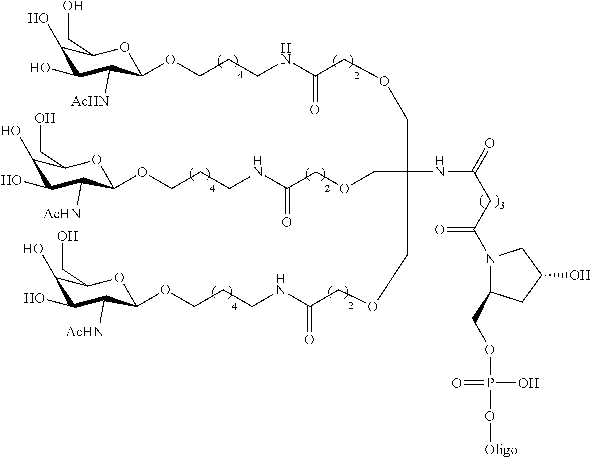

Representative United States patents, United States patent application publications, international patent application publications, and other publications that teach the preparation of certain of the above noted conjugate groups, oligomeric compounds comprising conjugate groups, tethers, conjugate linkers, branching groups, ligands, cleavable moieties as well as other modifications include without limitation, U.S. Pat. Nos. 5,994,517, 6,300,319, 6,660,720, 6,906,182, 7,262,177, 7,491,805, 8,106,022, 7,723,509, US 2006/0148740, US 2011/0123520, WO 2013/033230 and WO 2012/037254, Biessen et al., J. Med. Chem. 1995, 38, 1846-1852, Lee et al., Bioorganic & Medicinal Chemistry 2011, 19, 2494-2500, Rensen et al., J. Biol. Chem. 2001, 276, 37577-37584, Rensen et al., J. Med. Chem. 2004, 47, 5798-5808, Sliedregt et al., J. Med. Chem. 1999, 42, 609-618, and Valentijn et al., Tetrahedron, 1997, 53, 759-770.

In certain embodiments, oligomeric compounds comprise modified oligonucleotides comprising a gapmer and a conjugate group comprising at least one, two, or three GalNAc ligands. In certain embodiments antisense compounds and oligomeric compounds comprise a conjugate group found in any of the following references: Lee, Carbohydrate Research, 1978, 67, 509-514; Connolly et al., J Biol Chem, 1982, 257, 939-945; Pavia et al., Int J Pep Protein Res, 1983, 22, 539-548; Lee et al., Biochem, 1984, 23, 4255-4261; Lee et al., Glycoconjugate J, 1987, 4, 317-328; Toyokuni et al., Tetrahedron Lett, 1990, 31, 2673-2676; Biessen et al., J Med Chem, 1995, 38, 1538-1546; Valentijn et al., Tetrahedron, 1997, 53, 759-770; Kim et al., Tetrahedron Lett, 1997, 38, 3487-3490; Lee et al., Bioconjug Chem, 1997, 8, 762-765; Kato et al., Glycobiol, 2001, 11, 821-829; Rensen et al., J Blot Chem, 2001, 276, 37577-37584; Lee et al., Methods Enzymol, 2003, 362, 38-43; Westerlind et al., Glycoconj J, 2004, 21, 227-241; Lee et al., Bioorg Med Chem Lett, 2006, 16(19), 5132-5135; Maierhofer et al., Bioorg Med Chem, 2007, 15, 7661-7676; Khorev et al., Bioorg Med Chem, 2008, 16, 5216-5231; Lee et al., Bioorg Med Chem, 2011, 19, 2494-2500; Kornilova et al., Analyt Biochem, 2012, 425, 43-46; Pujol et al., Angew Chemie Int Ed Engl, 2012, 51, 7445-7448; Biessen et al., J Med Chem, 1995, 38, 1846-1852; Sliedregt et al., J Med Chem, 1999, 42, 609-618; Rensen et al., J Med Chem, 2004, 47, 5798-5808; Rensen et al., Arterioscler Thromb Vasc Biol, 2006, 26, 169-175; van Rossenberg et al., Gene Ther, 2004, 11, 457-464; Sato et al., J Am Chem Soc, 2004, 126, 14013-14022; Lee et al., J Org Chem, 2012, 77, 7564-7571; Biessen et al., FASEB J, 2000, 14, 1784-1792; Rajur et al., Bioconjug Chem, 1997, 8, 935-940; Duff et al., Methods Enzymol, 2000, 313, 297-321; Maier et al., Bioconjug Chem, 2003, 14, 18-29; Jayaprakash et al., Org Lett, 2010, 12, 5410-5413; Manoharan, Antisense Nucleic Acid Drug Dev, 2002, 12, 103-128; Merwin et al., Bioconjug Chem, 1994, 5, 612-620; Tomiya et al., Bioorg Med Chem, 2013, 21, 5275-5281; International applications WO1998/013381; WO2011/038356; WO1997/046098; WO2008/098788; WO2004/101619; WO2012/037254; WO2011/120053; WO2011/100131; WO2011/163121; WO2012/177947; WO2013/033230; WO2013/075035; WO2012/083185; WO2012/083046; WO2009/082607; WO2009/134487; WO2010/144740; WO2010/148013; WO1997/020563; WO2010/088537; WO2002/043771; WO2010/129709; WO2012/068187; WO2009/126933; WO2004/024757; WO2010/054406; WO2012/089352; WO2012/089602; WO2013/166121; WO2013/165816; U.S. Pat. Nos. 4,751,219; 8,552,163; 6,908,903; 7,262,177; 5,994,517; 6,300,319; 8,106,022; 7,491,805; 7,491,805; 7,582,744; 8,137,695; 6,383,812; 6,525,031; 6,660,720; 7,723,509; 8,541,548; 8,344,125; 8,313,772; 8,349,308; 8,450,467; 8,501,930; 8,158,601; 7,262,177; 6,906,182; 6,620,916; 8,435,491; 8,404,862; 7,851,615; Published U.S. Patent Application Publications US2011/0097264; US2011/0097265; US2013/0004427; US2005/0164235; US2006/0148740; US2008/0281044; 052010/0240730; US2003/0119724; US2006/0183886; US2008/0206869; US2011/0269814; US2009/0286973; US2011/0207799; US2012/0136042; US2012/0165393; US2008/0281041; US2009/0203135; US2012/0035115; US2012/0095075; US2012/0101148; US2012/0128760; US2012/0157509; US2012/0230938; US2013/0109817; US2013/0121954; US2013/0178512; US2013/0236968; US2011/0123520; US2003/0077829; US2008/0108801; and US2009/0203132.

In certain embodiments, compounds of the invention are single-stranded. In certain embodiments, oligomeric compounds are paired with a second oligonucleotide or oligomeric compound to form a duplex, which is double-stranded.

III. Certain Antisense Compounds

In certain embodiments, the present invention provides antisense compounds, which comprise or consist of an oligomeric compound comprising an antisense oligonucleotide, having a nucleobase sequences complementary to that of a target nucleic acid. In certain embodiments, antisense compounds are single-stranded. Such single-stranded antisense compounds typically comprise or consist of an oligomeric compound that comprises or consists of a modified oligonucleotide and optionally a conjugate group. In certain embodiments, antisense compounds are double-stranded. Such double-stranded antisense compounds comprise a first oligomeric compound having a region complementary to a target nucleic acid and a second oligomeric compound having a region complementary to the first oligomeric compound. The first oligomeric compound of such double stranded antisense compounds typically comprises or consists of a modified oligonucleotide and optionally a conjugate group. The oligonucleotide of the second oligomeric compound of such double-stranded antisense compound may be modified or unmodified. Either or both oligomeric compounds of a double-stranded antisense compound may comprise a conjugate group. The oligomeric compounds of double-stranded antisense compounds may include non-complementary overhanging nucleosides.

In certain embodiments, oligomeric compounds of antisense compounds are capable of hybridizing to a target nucleic acid, resulting in at least one antisense activity. In certain embodiments, antisense compounds selectively affect one or more target nucleic acid. Such selective antisense compounds comprises a nucleobase sequence that hybridizes to one or more target nucleic acid, resulting in one or more desired antisense activity and does not hybridize to one or more non-target nucleic acid or does not hybridize to one or more non-target nucleic acid in such a way that results in significant undesired antisense activity.

In certain antisense activities, hybridization of an antisense compound to a target nucleic acid results in recruitment of a protein that cleaves the target nucleic acid. For example, certain antisense compounds result in RNase H mediated cleavage of the target nucleic acid. RNase H is a cellular endonuclease that cleaves the RNA strand of an RNA:DNA duplex. The DNA in such an RNA:DNA duplex need not be unmodified DNA. In certain embodiments, the invention provides antisense compounds that are sufficiently “DNA-like” to elicit RNase H activity. Further, in certain embodiments, one or more non-DNA-like nucleoside in the gap of a gapmer is tolerated.

In certain antisense activities, an antisense compound or a portion of an antisense compound is loaded into an RNA-induced silencing complex (RISC), ultimately resulting in cleavage of the target nucleic acid. For example, certain antisense compounds result in cleavage of the target nucleic acid by Argonaute. Antisense compounds that are loaded into RISC are RNAi compounds. RNAi compounds may be double-stranded (siRNA) or single-stranded (ssRNA).

In certain embodiments, compounds comprising oligonucleotides having a gapmer nucleoside motif including one or more modified internucleoside linkages, having one of formulas I to XVI as described herein, have desirable properties compared to otherwise equivalent gapmers. In certain circumstances, it is desirable to identify gapmer motifs resulting in a favorable combination of potent antisense activity and relatively low toxicity. In certain embodiments, gapped oligomeric compounds of the present invention have a favorable therapeutic index (measure of potency divided by measure of toxicity).

In certain embodiments, hybridization of an antisense compound to a target nucleic acid does not result in recruitment of a protein that cleaves that target nucleic acid. In certain such embodiments, hybridization of the antisense compound to the target nucleic acid results in alteration of splicing of the target nucleic acid. In certain embodiments, hybridization of an antisense compound to a target nucleic acid results in inhibition of a binding interaction between the target nucleic acid and a protein or other nucleic acid. In certain such embodiments, hybridization of an antisense compound to a target nucleic acid results in alteration of translation of the target nucleic acid.

Antisense activities may be observed directly or indirectly. In certain embodiments, observation or detection of an antisense activity involves observation or detection of a change in an amount of a target nucleic acid or protein encoded by such target nucleic acid, a change in the ratio of splice variants of a nucleic acid or protein, and/or a phenotypic change in a cell or animal.

IV. Certain Target Nucleic Acids

In certain embodiments, antisense compounds comprise or consist of an oligonucleotide comprising a region that is complementary to a target nucleic acid. In certain embodiments, the target nucleic acid is an endogenous RNA molecule. In certain embodiments, the target nucleic acid encodes a protein. In certain such embodiments, the target nucleic acid is selected from: an mRNA and a pre-mRNA, including intronic, exonic and untranslated regions. In certain embodiments, the target RNA is an mRNA. In certain embodiments, the target nucleic acid is a pre-mRNA. In certain such embodiments, the target region is entirely within an intron. In certain embodiments, the target region spans an intron/exon junction. In certain embodiments, the target region is at least 50% within an intron.

In certain embodiments, the target nucleic acid is a non-coding RNA. In certain such embodiments, the target non-coding RNA is selected from: a long-non-coding RNA, a short non-coding RNA, an intronic RNA molecule, a snoRNA, a scaRNA, a microRNA (including pre-microRNA and mature microRNA), a ribosomal RNA, and promoter directed RNA. In certain embodiments, the target nucleic acid is a nucleic acid other than a mature mRNA. In certain embodiments, the target nucleic acid is a nucleic acid other than a mature mRNA or a microRNA. In certain embodiments, the target nucleic acid is a non-coding RNA other than a microRNA. In certain embodiments, the target nucleic acid is a non-coding RNA other than a microRNA or an intronic region of a pre-mRNA. In certain embodiments, the target nucleic acid is a long non-coding RNA. In certain embodiments, the target nucleic acid is a non-coding RNA associated with splicing of other pre-mRNAs. In certain embodiments, the target nucleic acid is a nuclear-retained non-coding RNA.

In certain embodiments, antisense compounds described herein are complementary to a target nucleic acid comprising a single-nucleotide polymorphism (SNP). In certain such embodiments, the antisense compound is capable of modulating expression of one allele of the SNP-containing target nucleic acid to a greater or lesser extent than it modulates another allele. In certain embodiments, an antisense compound hybridizes to a (SNP)-containing target nucleic acid at the single-nucleotide polymorphism site.

In certain embodiments, antisense compounds are at least partially complementary to more than one target nucleic acid. For example, antisense compounds of the present invention may mimic microRNAs, which typically bind to multiple targets.

A. Complementarity/Mismatches to the Target Nucleic Acid

In certain embodiments, antisense compounds comprise antisense oligonucleotides that are complementary to the target nucleic acid over the entire length of the oligonucleotide. In certain embodiments, such oligonucleotides are 99% complementary to the target nucleic acid. In certain embodiments, such oligonucleotides are 95% complementary to the target nucleic acid. In certain embodiments, such oligonucleotides are 90% complementary to the target nucleic acid. In certain embodiments, such oligonucleotides are 85% complementary to the target nucleic acid. In certain embodiments, such oligonucleotides are 80% complementary to the target nucleic acid. In certain embodiments, antisense oligonucleotides are at least 80% complementary to the target nucleic acid over the entire length of the oligonucleotide and comprise a region that is 100% or fully complementary to a target nucleic acid. In certain such embodiments, the region of full complementarity is from 6 to 20 nucleobases in length. In certain such embodiments, the region of full complementarity is from 10 to 18 nucleobases in length. In certain such embodiments, the region of full complementarity is from 18 to 20 nucleobases in length.

In certain embodiments, the oligomeric compounds of antisense compounds comprise one or more mismatched nucleobases relative to the target nucleic acid. In certain such embodiments, antisense activity against the target is reduced by such mismatch, but activity against a non-target is reduced by a greater amount. Thus, in certain such embodiments selectivity of the antisense compound is improved. In certain embodiments, the mismatch is specifically positioned within an oligonucleotide having a gapmer motif. In certain such embodiments, the mismatch is at position 1, 2, 3, 4, 5, 6, 7, or 8 from the 5′-end of the gap region (5′-gap junction). In certain such embodiments, the mismatch is at position 9, 8, 7, 6, 5, 4, 3, 2, 1 from the 3′-end of the gap region (3a′-gap junction). In certain such embodiments, the mismatch is at position 1, 2, 3, or 4 from the 5′-end of the wing region. In certain such embodiments, the mismatch is at position 4, 3, 2, or 1 from the 3′-end of the wing region.

B. Certain Target Nucleic Acids in Certain Tissues

In certain embodiments, antisense compounds comprise or consist of an oligonucleotide comprising a region that is complementary to a target nucleic acid, wherein the target nucleic acid is expressed in an extra-hepatic tissue. Extra-hepatic tissues include, but are not limited to: skeletal muscle, cardiac muscle, smooth muscle, adipose, white adipose, spleen, bone, intestine, adrenal, testes, ovary, pancreas, pituitary, prostate, skin, uterus, bladder, brain, glomerulus, distal tubular epithelium, breast, lung, heart, kidney, ganglion, frontal cortex, spinal cord, trigeminal ganglia, sciatic nerve, dorsal root ganglion, epididymal fat, diaphragm, pancreas, and colon.

V. Certain Pharmaceutical Compositions

In certain embodiments, the present invention provides pharmaceutical compositions comprising one or more antisense compound or a salt thereof. In certain such embodiments, the pharmaceutical composition comprises a suitable pharmaceutically acceptable diluent or carrier. In certain embodiments, a pharmaceutical composition comprises a sterile saline solution and one or more antisense compound. In certain embodiments, such pharmaceutical composition consists of a sterile saline solution and one or more antisense compound. In certain embodiments, the sterile saline is pharmaceutical grade saline. In certain embodiments, a pharmaceutical composition comprises one or more antisense compound and sterile water. In certain embodiments, a pharmaceutical composition consists of one antisense compound and sterile water. In certain embodiments, the sterile water is pharmaceutical grade water. In certain embodiments, a pharmaceutical composition comprises one or more antisense compound and phosphate-buffered saline (PBS). In certain embodiments, a pharmaceutical composition consists of one or more antisense compound and sterile PBS. In certain embodiments, the sterile PBS is pharmaceutical grade PBS.

In certain embodiments, pharmaceutical compositions comprise one or more or antisense compound and one or more excipients. In certain such embodiments, excipients are selected from water, salt solutions, alcohol, polyethylene glycols, gelatin, lactose, amylase, magnesium stearate, talc, silicic acid, viscous paraffin, hydroxymethylcellulose and polyvinylpyrrolidone.

In certain embodiments, antisense compounds may be admixed with pharmaceutically acceptable active and/or inert substances for the preparation of pharmaceutical compositions or formulations. Compositions and methods for the formulation of pharmaceutical compositions depend on a number of criteria, including, but not limited to, route of administration, extent of disease, or dose to be administered.

In certain embodiments, pharmaceutical compositions comprising an antisense compound encompass any pharmaceutically acceptable salts of the antisense compound, esters of the antisense compound, or salts of such esters. In certain embodiments, pharmaceutical compositions comprising antisense compounds comprising one or more antisense oligonucleotide, upon administration to an animal, including a human, are capable of providing (directly or indirectly) the biologically active metabolite or residue thereof. Accordingly, for example, the disclosure is also drawn to pharmaceutically acceptable salts of antisense compounds, prodrugs, pharmaceutically acceptable salts of such prodrugs, and other bioequivalents. Suitable pharmaceutically acceptable salts include, but are not limited to, sodium and potassium salts. In certain embodiments, prodrugs comprise one or more conjugate group attached to an oligonucleotide, wherein the conjugate group is cleaved by endogenous nucleases within the body.

Lipid moieties have been used in nucleic acid therapies in a variety of methods. In certain such methods, the nucleic acid, such as an antisense compound, is introduced into preformed liposomes or lipoplexes made of mixtures of cationic lipids and neutral lipids. In certain methods, DNA complexes with mono- or poly-cationic lipids are formed without the presence of a neutral lipid. In certain embodiments, a lipid moiety is selected to increase distribution of a pharmaceutical agent to a particular cell or tissue. In certain embodiments, a lipid moiety is selected to increase distribution of a pharmaceutical agent to fat tissue. In certain embodiments, a lipid moiety is selected to increase distribution of a pharmaceutical agent to muscle tissue.

In certain embodiments, pharmaceutical compositions comprise a delivery system. Examples of delivery systems include, but are not limited to, liposomes and emulsions. Certain delivery systems are useful for preparing certain pharmaceutical compositions including those comprising hydrophobic compounds. In certain embodiments, certain organic solvents such as dimethylsulfoxide are used.

In certain embodiments, pharmaceutical compositions comprise one or more tissue-specific delivery molecules designed to deliver the one or more pharmaceutical agents of the present invention to specific tissues or cell types. For example, in certain embodiments, pharmaceutical compositions include liposomes coated with a tissue-specific antibody.

In certain embodiments, pharmaceutical compositions comprise a co-solvent system. Certain of such co-solvent systems comprise, for example, benzyl alcohol, a nonpolar surfactant, a water-miscible organic polymer, and an aqueous phase. In certain embodiments, such co-solvent systems are used for hydrophobic compounds. A non-limiting example of such a co-solvent system is the VPD co-solvent system, which is a solution of absolute ethanol comprising 3% w/v benzyl alcohol, 8% w/v of the nonpolar surfactant Polysorbate80™ and 65% w/v polyethylene glycol 300. The proportions of such co-solvent systems may be varied considerably without significantly altering their solubility and toxicity characteristics. Furthermore, the identity of co-solvent components may be varied: for example, other surfactants may be used instead of Polysorbate 80™; the fraction size of polyethylene glycol may be varied; other biocompatible polymers may replace polyethylene glycol, e.g., polyvinyl pyrrolidone; and other sugars or polysaccharides may substitute for dextrose.

In certain embodiments, pharmaceutical compositions are prepared for oral administration. In certain embodiments, pharmaceutical compositions are prepared for buccal administration. In certain embodiments, a pharmaceutical composition is prepared for administration by injection (e.g., intravenous, subcutaneous, intramuscular, etc.). In certain of such embodiments, a pharmaceutical composition comprises a carrier and is formulated in aqueous solution, such as water or physiologically compatible buffers such as Hanks's solution, Ringer's solution, or physiological saline buffer. In certain embodiments, other ingredients are included (e.g., ingredients that aid in solubility or serve as preservatives). In certain embodiments, injectable suspensions are prepared using appropriate liquid carriers, suspending agents and the like. Certain pharmaceutical compositions for injection are presented in unit dosage form, e.g., in ampoules or in multi-dose containers. Certain pharmaceutical compositions for injection are suspensions, solutions or emulsions in oily or aqueous vehicles, and may contain formulatory agents such as suspending, stabilizing and/or dispersing agents. Certain solvents suitable for use in pharmaceutical compositions for injection include, but are not limited to, lipophilic solvents and fatty oils, such as sesame oil, synthetic fatty acid esters, such as ethyl oleate or triglycerides, and liposomes. Aqueous injection suspensions may contain.

### Nonlimiting Disclosure and Incorporation by Reference

Each of the literature and patent publications listed herein is incorporated by reference in its entirety. While certain compounds, compositions and methods described herein have been described with specificity in accordance with certain embodiments, the following examples serve only to illustrate the compounds described herein and are not intended to limit the same. Each of the references, GenBank accession numbers, and the like recited in the present application is incorporated herein by reference in its entirety.

Although the sequence listing accompanying this filing identifies each sequence as either “RNA” or “DNA” as required, in reality, those sequences may be modified with any combination of chemical modifications. One of skill in the art will readily appreciate that such designation as “RNA” or “DNA” to describe modified oligonucleotides is, in certain instances, arbitrary. For example, an oligonucleotide comprising a nucleoside comprising a 2′-OH sugar moiety and a thymine base could be described as a DNA having a modified sugar (2′-OH in place of one 2′-H of DNA) or as an RNA having a modified base (thymine (methylated uracil) in place of a uracil of RNA). Accordingly, nucleic acid sequences provided herein, including, but not limited to those in the sequence listing, are intended to encompass nucleic acids containing any combination of natural or modified RNA and/or DNA, including, but not limited to such nucleic acids having modified nucleobases. By way of further example and without limitation, an oligomeric compound having the nucleobase sequence “ATCGATCG” encompasses any oligomeric compounds having such nucleobase sequence, whether modified or unmodified, including, but not limited to, such compounds comprising RNA bases, such as those having sequence “AUCGAUCG” and those having some DNA bases and some RNA bases such as “AUCGATCG” and oligomeric compounds having other modified nucleobases, such as “AT^{m}CGAUCG,” wherein ^{m}C indicates a cytosine base comprising a methyl group at the 5-position.

Certain compounds described herein (e.g., modified oligonucleotides) have one or more asymmetric center and thus give rise to enantiomers, diastereomers, and other stereoisomeric configurations that may be defined, in terms of absolute stereochemistry, as (R) or (S), as α or β such as for sugar anomers, or as (D) or (L), such as for amino acids, etc. Included in the compounds provided herein are all such possible isomers, including their racemic and optically pure forms, unless specified otherwise. Likewise, all cis- and trans-isomers and tautomeric forms are also included unless otherwise indicated. Unless otherwise indicated, compounds described herein are intended to include corresponding salt forms.

### EXAMPLES

### Example 1

### Synthesis of Nucleoside Phosphoramidites

The preparation of nucleoside phosphoramidites is performed following procedures that are illustrated herein and in the art such as but not limited to U.S. Pat. No. 6,426,220 and published PCT WO 02/36743.

### Example 2

### Synthesis of Oligomeric Compounds

The oligomeric compounds used in accordance with this invention may be conveniently and routinely made through the well-known technique of solid phase synthesis. Equipment for such synthesis is sold by several vendors including, for example, Applied Biosystems (Foster City, Calif.). Any other means for such synthesis known in the art may additionally or alternatively be employed. It is well known to use similar techniques to prepare oligonucleotides such as alkylated derivatives and those having phosphorothioate linkages.

Oligomeric compounds: Unsubstituted and substituted phosphodiester (P═O) oligomeric compounds, including without limitation, oligonucleotides can be synthesized on an automated DNA synthesizer (Applied Biosystems model 394) using standard phosphoramidite chemistry with oxidation by iodine.

In certain embodiments, phosphorothioate internucleoside linkages (P═S) are synthesized similar to phosphodiester internucleoside linkages with the following exceptions: thiation is effected by utilizing a 10% w/v solution of 3,H-1,2-benzodithiole-3-one 1,1-dioxide in acetonitrile for the oxidation of the phosphite linkages. The thiation reaction step time is increased to 180 sec and preceded by the normal capping step. After cleavage from the CPG column and deblocking in concentrated ammonium hydroxide at 55° C. (12-16 hr), the oligomeric compounds are recovered by precipitating with greater than 3 volumes of ethanol from a 1 M NH₄OAc solution. Phosphinate internucleoside linkages can be prepared as described in U.S. Pat. No. 5,508,270.

Alkyl phosphonate internucleoside linkages can be prepared as described in U.S. Pat. No. 4,469,863.

3′-Deoxy-3′-methylene phosphonate internucleoside linkages can be prepared as described in U.S. Pat. No. 5,610,289 or 5,625,050.

Phosphoramidite internucleoside linkages can be prepared as described in U.S. Pat. No. 5,256,775 or 5,366,878.

Alkylphosphonothioate internucleoside linkages can be prepared as described in published PCT applications PCT/US94/00902 and PCT/US93/06976 (published as WO 94/17093 and WO 94/02499, respectively).

3′-Deoxy-3′-amino phosphoramidate internucleoside linkages can be prepared as described in U.S. Pat. No. 5,476,925.

Phosphotriester internucleoside linkages can be prepared as described in U.S. Pat. No. 5,023,243.

Borano phosphate internucleoside linkages can be prepared as described in U.S. Pat. Nos. 5,130,302 and 5,177,198.

Oligomeric compounds having one or more non-phosphorus containing internucleoside linkages including without limitation methylenemethylimino linked oligonucleosides, also identified as MMI linked oligonucleosides, methylenedimethylhydrazo linked oligonucleosides, also identified as MDH linked oligonucleosides, methylenecarbonylamino linked oligonucleosides, also identified as amide-3 linked oligonucleosides, and methyleneaminocarbonyl linked oligonucleosides, also identified as amide-4 linked oligonucleosides, as well as mixed backbone oligomeric compounds having, for instance, alternating MMI and P═O or P═S linkages can be prepared as described in U.S. Pat. Nos. 5,378,825, 5,386,023, 5,489,677, 5,602,240 and 5,610,289.

Formacetal and thioformacetal internucleoside linkages can be prepared as described in U.S. Pat. Nos. 5,264,562 and 5,264,564.

Ethylene oxide internucleoside linkages can be prepared as described in U.S. Pat. No. 5,223,618.

### Example 3

### Isolation and Purification of Oligomeric Compounds

After cleavage from the controlled pore glass solid support or other support medium and deblocking in concentrated ammonium hydroxide at 55° C. for 12-16 hours, the oligomeric compounds, including without limitation oligonucleotides and oligonucleosides, are recovered by precipitation out of 1 M NH₄OAc with >3 volumes of ethanol. Synthesized oligomeric compounds are analyzed by electrospray mass spectroscopy (molecular weight determination) and by capillary gel electrophoresis. The relative amounts of phosphorothioate and phosphodiester linkages obtained in the synthesis is determined by the ratio of correct molecular weight relative to the −16 amu product (+/−32+/−48). For some studies oligomeric compounds are purified by HPLC, as described by Chiang et al., J. Biol. Chem. 1991, 266(27), 18162-18171. Results obtained with HPLC-purified material are generally similar to those obtained with non-HPLC purified material.

### Example 4

### Synthesis of Oligomeric Compounds Using the 96 Well Plate Format

Oligomeric compounds, including without limitation oligonucleotides, can be synthesized via solid phase P(III) phosphoramidite chemistry on an automated synthesizer capable of assembling 96 sequences simultaneously in a 96-well format. Phosphodiester internucleoside linkages are afforded by oxidation with aqueous iodine. Phosphorothioate internucleoside linkages are generated by sulfurization utilizing 3,H-1,2 benzodithiole-3-one 1,1 dioxide (Beaucage Reagent) in anhydrous acetonitrile. Standard base-protected beta-cyanoethyl-diiso-propyl phosphoramidites can be purchased from commercial vendors (e.g. PE-Applied Biosystems, Foster City, Calif., or Pharmacia, Piscataway, N.J.). Non-standard nucleosides are synthesized as per standard or patented methods and can be functionalized as base protected beta-cyanoethyldiisopropyl phosphoramidites.

Oligomeric compounds can be cleaved from support and deprotected with concentrated NH₄OH at elevated temperature (55-60° C.) for 12-16 hours and the released product then dried in vacuo. The dried product is then re-suspended in sterile water to afford a master plate from which all analytical and test plate samples are then diluted utilizing robotic pipettors.

### Example 5

### Analysis of Oligomeric Compounds Using the 96-Well Plate Format

The concentration of oligomeric compounds in each well can be assessed by dilution of samples and UV absorption spectroscopy. The full-length integrity of the individual products can be evaluated by capillary electrophoresis (CE) in either the 96-well format (Beckman P/ACE™ MDQ) or, for individually prepared samples, on a commercial CE apparatus (e.g., Beckman P/ACE™ 5000, ABI 270). Base and backbone composition is confirmed by mass analysis of the oligomeric compounds utilizing electrospray-mass spectroscopy. All assay test plates are diluted from the master plate using single and multi-channel robotic pipettors. Plates are judged to be acceptable if at least 85% of the oligomeric compounds on the plate are at least 85% full length.

### Example 6

In Vitro Treatment of Cells with Oligomeric Compounds

The effect of oligomeric compounds on target nucleic acid expression is tested in any of a variety of cell types provided that the target nucleic acid is present at measurable levels. This can be routinely determined using, for example, PCR or Northern blot analysis. Cell lines derived from multiple tissues and species can be obtained from American Type Culture Collection (ATCC, Manassas, Va.).

The following cell type is provided for illustrative purposes, but other cell types can be routinely used, provided that the target is expressed in the cell type chosen. This can be readily determined by methods routine in the art, for example Northern blot analysis, ribonuclease protection assays or RT-PCR.

b.END cells: The mouse brain endothelial cell line b.END was obtained from Dr. Werner Risau at the Max Plank Institute (Bad Nauheim, Germany). b.END cells are routinely cultured in DMEM, high glucose (Invitrogen Life Technologies, Carlsbad, Calif.) supplemented with 10% fetal bovine serum (Invitrogen Life Technologies, Carlsbad, Calif.). Cells are routinely passaged by trypsinization and dilution when they reached approximately 90% confluence. Cells are seeded into 96-well plates (Falcon-Primaria #353872, BD Biosciences, Bedford, Mass.) at a density of approximately 3000 cells/well for uses including but not limited to oligomeric compound transfection experiments.

Experiments involving treatment of cells with oligomeric compounds:

When cells reach appropriate confluency, they are treated with oligomeric compounds using a transfection method as described.

LIPOFECTIN™

When cells reached 65-75% confluency, they are treated with one or more oligomeric compounds. The oligomeric compound is mixed with LIPOFECTIN™ Invitrogen Life Technologies Carlsbad, Calif.) in Opti-MEM™-1 reduced serum medium (Invitrogen Life Technologies, Carlsbad, Calif.) to achieve the desired concentration of the oligomeric compound(s) and a LIPOFECTIN™ concentration of 2.5 or 3 μg/mL per 100 nM oligomeric compound(s). This transfection mixture is incubated at room temperature for approximately 0.5 hours. For cells grown in 96-well plates, wells are washed once with 100 μL OPTI-MEM™-1 and then treated with 130 μL of the transfection mixture. Cells grown in 24-well plates or other standard tissue culture plates are treated similarly, using appropriate volumes of medium and oligomeric compound(s). Cells are treated and data are obtained in duplicate or triplicate. After approximately 4-7 hours of treatment at 37° C., the medium containing the transfection mixture is replaced with fresh culture medium. Cells are harvested 16-24 hours after treatment with oligomeric compound(s).

Other suitable transfection reagents known in the art include, but are not limited to, CYTOFECTIN™, LIPOFECTAMINE™, OLIGOFECTAMINE™, and FUGENE™. Other suitable transfection methods known in the art include, but are not limited to, electroporation.

### Example 7

Real-Time Quantitative PCR Analysis of Target mRNA Levels

Quantitation of target mRNA levels is accomplished by real-time quantitative PCR using the ABI PRISM™ 7600, 7700, or 7900 Sequence Detection System (PE-Applied Biosystems, Foster City, Calif.) according to manufacturer's instructions. This is a closed-tube, non-gel-based, fluorescence detection system which allows high-throughput quantitation of polymerase chain reaction (PCR) products in real-time. As opposed to standard PCR in which amplification products are quantitated after the PCR is completed, products in real-time quantitative PCR are quantitated as they accumulate. This is accomplished by including in the PCR reaction an oligonucleotide probe that anneals specifically between the forward and reverse PCR primers, and contains two fluorescent dyes. A reporter dye (e.g., FAM or JOE, obtained from either PE-Applied Biosystems, Foster City, Calif., Operon Technologies Inc., Alameda, Calif. or Integrated DNA Technologies Inc., Coralville, Iowa) is attached to the 5′ end of the probe and a quencher dye (e.g., TAN/IRA, obtained from either PE-Applied Biosystems, Foster City, Calif., Operon Technologies Inc., Alameda, Calif. or Integrated DNA Technologies Inc., Coralville, Iowa) is attached to the 3′ end of the probe. When the probe and dyes are intact, reporter dye emission is quenched by the proximity of the 3′ quencher dye. During amplification, annealing of the probe to the target sequence creates a substrate that can be cleaved by the 5′-exonuclease activity of Taq polymerase. During the extension phase of the PCR amplification cycle, cleavage of the probe by Taq polymerase releases the reporter dye from the remainder of the probe (and hence from the quencher moiety) and a sequence-specific fluorescent signal is generated. With each cycle, additional reporter dye molecules are cleaved from their respective probes, and the fluorescence intensity is monitored at regular intervals by laser optics built into the ABI PRISM™ Sequence Detection System. In each assay, a series of parallel reactions containing serial dilutions of mRNA from untreated control samples generates a standard curve that is used to quantitate the percent inhibition after antisense oligonucleotide treatment of test samples.

Prior to quantitative PCR analysis, primer-probe sets specific to the target gene being measured are evaluated for their ability to be “multiplexed” with a GAPDH amplification reaction. In multiplexing, both the target gene and the internal standard gene GAPDH are amplified concurrently in a single sample. In this analysis, mRNA isolated from untreated cells is serially diluted. Each dilution is amplified in the presence of primer-probe sets specific for GAPDH only, target gene only (“single-plexing”), or both (multiplexing). Following PCR amplification, standard curves of GAPDH and target mRNA signal as a function of dilution are generated from both the single-plexed and multiplexed samples. If both the slope and correlation coefficient of the GAPDH and target signals generated from the multiplexed samples fall within 10% of their corresponding values generated from the single-plexed samples, the primer-probe set specific for that target is deemed multiplexable. Other methods of PCR are also known in the art.

RT and PCR reagents are obtained from Invitrogen Life Technologies (Carlsbad, Calif.). RT, real-time PCR is carried out by adding 20 μL PCR cocktail (2.5×PCR buffer minus MgCl₂, 6.6 mM MgCl₂, 375 μM each of dATP, dCTP, dCTP and dGTP, 375 nM each of forward primer and reverse primer, 125 nM of probe, 4 Units RNAse inhibitor, 1.25 Units PLATINUM® Taq, 5 Units MuLV reverse transcriptase, and 2.5×ROX dye) to 96-well plates containing 30 μL total RNA solution (20-200 ng). The RT reaction is carried out by incubation for 30 minutes at 48° C. Following a 10 minute incubation at 95° C. to activate the PLATINUM® Taq, 40 cycles of a two-step PCR protocol are carried out: 95° C. for 15 seconds (denaturation) followed by 60° C. for 1.5 minutes (annealing/-extension).

Gene target quantities obtained by RT, real-time PCR are normalized using either the expression level of GAPDH, a gene whose expression is constant, or by quantifying total RNA using RIBOGREEN™ (Molecular Probes, Inc. Eugene, Oreg.). GAPDH expression is quantified by real time RT-PCR, by being run simultaneously with the target, multiplexing, or separately. Total RNA is quantified using RiboGreen™ RNA quantification reagent (Molecular Probes, Inc. Eugene, Oreg.). Methods of RNA quantification by RIBOGREEN™ are taught in Jones, L. J., et al, (Analytical Biochemistry, 1998, 265, 368-374).

In this assay, 170 μL of RIBOGREEN™ working reagent (RIBOGREEN™ reagent diluted 1:350 in 10 mM Tris-HCl, 1 mM EDTA, pH 7.5) is pipetted into a 96-well plate containing 30 μL purified, cellular RNA. The plate is read in a CytoFluor 4000 (PE Applied Biosystems) with excitation at 485 nm and emission at 530 nm.

### Example 8

### Analysis of Inhibition of Target Expression

Antisense modulation of a target expression can be assayed in a variety of ways known in the art. For example, a target mRNA levels can be quantitated by, e.g., Northern blot analysis, competitive polymerase chain reaction (PCR), or real-time PCR. Real-time quantitative PCR is presently desired. RNA analysis can be performed on total cellular RNA or poly(A)+ mRNA. One method of RNA analysis of the present disclosure is the use of total cellular RNA as described in other examples herein. Methods of RNA isolation are well known in the art. Northern blot analysis is also routine in the art. Real-time quantitative (PCR) can be conveniently accomplished using the commercially available ABI PRISM™ 7600, 7700, or 7900 Sequence Detection System, available from PE-Applied Biosystems, Foster City, Calif. and used according to manufacturer's instructions.

Protein levels of a target can be quantitated in a variety of ways well known in the art, such as immunoprecipitation, Western blot analysis (immunoblotting), enzyme-linked immunosorbent assay (ELISA) or fluorescence-activated cell sorting (FACS). Antibodies directed to a target can be identified and obtained from a variety of sources, such as the MSRS catalog of antibodies (Aerie Corporation, Birmingham, Mich.), or can be prepared via conventional monoclonal or polyclonal antibody generation methods well known in the art. Methods for preparation of polyclonal antisera are taught in, for example, Ausubel, F. M. et al., Current Protocols in Molecular Biology, Volume 2, pp. 11.12.1-11.12.9, John Wiley & Sons, Inc., 1997. Preparation of monoclonal antibodies is taught in, for example, Ausubel, F. M. et al., Current Protocols in Molecular Biology, Volume 2, pp. 11.4.1-11.11.5, John Wiley & Sons, Inc., 1997.

Immunoprecipitation methods are standard in the art and can be found at, for example, Ausubel, F. M. et al., Current Protocols in Molecular Biology, Volume 2, pp. 10.16.1-10.16.11, John Wiley & Sons, Inc., 1998. Western blot (immunoblot) analysis is standard in the art and can be found at, for example, Ausubel, F. M. et al., Current Protocols in Molecular Biology, Volume 2, pp. 10.8.1-10.8.21, John Wiley & Sons, Inc., 1997. Enzyme-linked immunosorbent assays (ELISA) are standard in the art and can be found at, for example, Ausubel, F. M. et al., Current Protocols in Molecular Biology, Volume 2, pp. 11.2.1-11.2.22, John Wiley & Sons, Inc., 1991.

### Example 9

### Design of Phenotypic Assays and In Vivo Studies for the Use of Target Inhibitors

### Phenotypic Assays

Once target inhibitors have been identified by the methods disclosed herein, the oligomeric compounds are further investigated in one or more phenotypic assays, each having measurable endpoints predictive of efficacy in the treatment of a particular disease state or condition.

Phenotypic assays, kits and reagents for their use are well known to those skilled in the art and are herein used to investigate the role and/or association of a target in health and disease. Representative phenotypic assays, which can be purchased from any one of several commercial vendors, include those for determining cell viability, cytotoxicity, proliferation or cell survival (Molecular Probes, Eugene, Oreg.; PerkinElmer, Boston, Mass.), protein-based assays including enzymatic assays (Panvera, LLC, Madison, Wis.; BD Biosciences, Franklin Lakes, N.J.; Oncogene Research Products, San Diego, Calif.), cell regulation, signal transduction, inflammation, oxidative processes and apoptosis (Assay Designs Inc., Ann Arbor, Mich.), triglyceride accumulation (Sigma-Aldrich, St. Louis, Mo.), angiogenesis assays, tube formation assays, cytokine and hormone assays and metabolic assays (Chemicon International Inc., Temecula, Calif.; Amersham Biosciences, Piscataway, N.J.).

In one non-limiting example, cells determined to be appropriate for a particular phenotypic assay (i.e., MCF-7 cells selected for breast cancer studies; adipocytes for obesity studies) are treated with a target inhibitors identified from the in vitro studies as well as control compounds at optimal concentrations which are determined by the methods described above. At the end of the treatment period, treated and untreated cells are analyzed by one or more methods specific for the assay to determine phenotypic outcomes and endpoints.

Phenotypic endpoints include changes in cell morphology over time or treatment dose as well as changes in levels of cellular components such as proteins, lipids, nucleic acids, hormones, saccharides or metals. Measurements of cellular status which include pH, stage of the cell cycle, intake or excretion of biological indicators by the cell, are also endpoints of interest.

Measurement of the expression of one or more of the genes of the cell after treatment is also used as an indicator of the efficacy or potency of the target inhibitors. Hallmark genes, or those genes suspected to be associated with a specific disease state, condition, or phenotype, are measured in both treated and untreated cells.

### In Vivo Studies

The individual subjects of the in vivo studies described herein are warm-blooded vertebrate animals, which includes humans.

### Example 10

### RNA Isolation

Poly(A)+ mRNA Isolation

Poly(A)+ mRNA is isolated according to Miura et al., (Clin. Chem., 1996, 42, 1758-1764). Other methods for poly(A)+ mRNA isolation are routine in the art. Briefly, for cells grown on 96-well plates, growth medium is removed from the cells and each well is washed with 200 μL cold PBS. 60 μL lysis buffer (10 mM Tris-HCl, pH 7.6, 1 mM EDTA, 0.5 M NaCl, 0.5% NP-40, 20 mM vanadyl-ribonucleoside complex) is added to each well, the plate is gently agitated and then incubated at room temperature for five minutes. 55 μL of lysate is transferred to Oligo d(T) coated 96-well plates (AGCT Inc., Irvine Calif.). Plates are incubated for 60 minutes at room temperature, washed 3 times with 200 μL of wash buffer (10 mM Tris-HCl pH 7.6, 1 mM EDTA, 0.3 M NaCl). After the final wash, the plate is blotted on paper towels to remove excess wash buffer and then air-dried for 5 minutes. 60 μL of elution buffer (5 mM Tris-HCl pH 7.6), preheated to 70° C., is added to each well, the plate is incubated on a 90° C. hot plate for 5 minutes, and the eluate is then transferred to a fresh 96-well plate.

Cells grown on 100 mm or other standard plates may be treated similarly, using appropriate volumes of all solutions.

### Total RNA Isolation

Total RNA is isolated using an RNEASY 96™ kit and buffers purchased from Qiagen Inc. (Valencia, Calif.) following the manufacturer's recommended procedures. Briefly, for cells grown on 96-well plates, growth medium is removed from the cells and each well is washed with 200 μL cold PBS. 150 μL Buffer RLT is added to each well and the plate vigorously agitated for 20 seconds. 150 μL of 70% ethanol is then added to each well and the contents mixed by pipetting three times up and down. The samples are then transferred to the RNEASY 96™ well plate attached to a QIAVAC™ manifold fitted with a waste collection tray and attached to a vacuum source. Vacuum is applied for 1 minute. 500 μL of Buffer RW1 is added to each well of the RNEASY 96™ plate and incubated for 15 minutes and the vacuum is again applied for 1 minute. An additional 500 μL of Buffer RW1 is added to each well of the RNEASY 96™ plate and the vacuum is applied for 2 minutes. 1 mL of Buffer RPE is then added to each well of the RNEASY 96™ plate and the vacuum applied for a period of 90 seconds. The Buffer RPE wash is then repeated and the vacuum is applied for an additional 3 minutes. The plate is then removed from the QIAVAC™ manifold and blotted dry on paper towels. The plate is then re-attached to the QIAVAC™ manifold fitted with a collection tube rack containing 1.2 mL collection tubes. RNA is then eluted by pipetting 140 μL of RNAse free water into each well, incubating 1 minute, and then applying the vacuum for 3 minutes.

The repetitive pipetting and elution steps may be automated using a QIAGEN Bio-Robot 9604 (Qiagen, Inc., Valencia Calif.). Essentially, after lysing of the cells on the culture plate, the plate is transferred to the robot deck where the pipetting, DNase treatment and elution steps are carried out.

### Example 11

### Target-Specific Primers and Probes

Probes and primers may be designed to hybridize to a target sequence, using published sequence information.

For example, for human PTEN, the following primer-probe set was designed using published sequence information (GENBANK™ accession number U92436.1, SEQ ID NO: 5).

Forward primer:
(SEQ ID NO: 6
AATGGCTAAGTGAAGATGACAATCAT
Reverse primer:
(SEQ ID NO: 7)
TGCACATATCATTACACCAGTTCGT

And the PCR probe:

FAM-TTGCAGCAATTCACTGTAAAGCTGGAAAGG-TAMRA (SEQ ID NO: 8), where FAM is the fluorescent dye and TAMRA is the quencher dye.

### Example 12

### Western Blot Analysis of Target Protein Levels

Western blot analysis (immunoblot analysis) is carried out using standard methods. Cells are harvested 16-20 h after oligonucleotide treatment, washed once with PBS, suspended in Laemmli buffer (100 μL/well), boiled for 5 minutes and loaded on a 16% SDS-PAGE gel. Gels are run for 1.5 hours at 150 V, and transferred to membrane for western blotting. Appropriate primary antibody directed to a target is used, with a radiolabeled or fluorescently labeled secondary antibody directed against the primary antibody species. Bands are visualized using a PHOSPHORIMAGER™ (Molecular Dynamics, Sunnyvale Calif.).

### Example 13

### Synthesis of DMT Protected Phenyl Diisopropylaminophosphonamidite Nucleosides

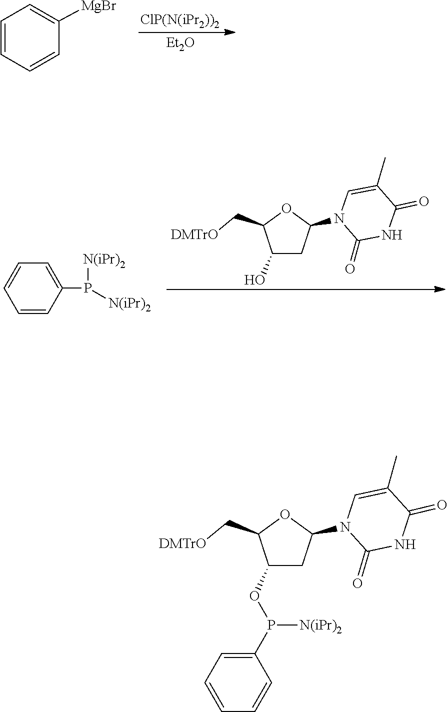

A Grignard reagent is selected and reacted with bis(diisopropylamino)chlorophosphine and then the product is reacted with a selected DMT protected nucleoside (for example thymidine). The resulting reactive phosphorus group will form the corresponding phosphonate internucleoside linkage (phenylphosphonate in this example) when reacted with a free hydroxyl of a nucleoside or oligonucleotide, generally the 5′-hydroxyl, during routine oligonucleotide synthesis.

### Example 14

### Synthesis of DMT Protected Primary Alkyl Diisopropylaminophosphonamidite Nucleosides

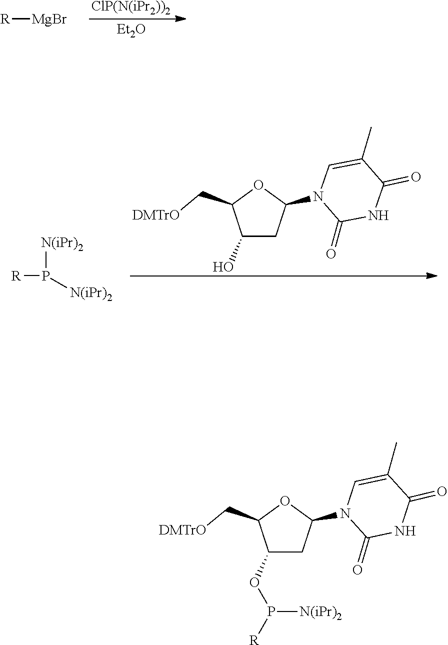

The appropriate primary alkyl Grignard reagent is selected and reacted with bis(diisopropyl-amino)chlorophosphine and then the product is reacted with a selected DMT protected nucleoside (for example thymidine). The resulting reactive phosphorus group will form the corresponding phosphonate internucleoside linkage (R-phosphonate in this example) when reacted with a free hydroxyl of a nucleoside or oligonucleotide, generally the 5′-hydroxyl, during routine oligonucleotide synthesis.

Primary alkyl diisopropylaminophosphonamidite thymidine nucleosides were prepared wherein R is propyl, isobutyl and pentyl (spectra for each consistent with structure).

### Example 15

### Synthesis of DMT Protected Cyclohexyl Diisopropylaminophosphonamidite Nucleosides

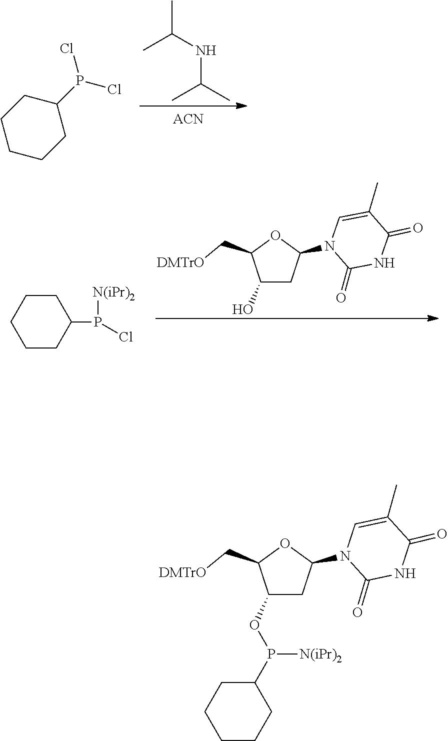

An appropriate dichlorophosphine is selected and reacted with diisopropylamine and then the product is reacted with a selected DMT protected nucleoside (for example thymidine). The resulting reactive phosphorus group will form the corresponding phosphonate internucleoside linkage (cyclohexylphosphonate in this example) when reacted with a free hydroxyl of a nucleoside or oligonucleotide, generally the 5′-hydroxyl, during routine oligonucleotide synthesis.

### Example 16

### Synthesis of DMT Protected Secondary Alkyl Diisopropylaminophosphonamidite Nucleosides

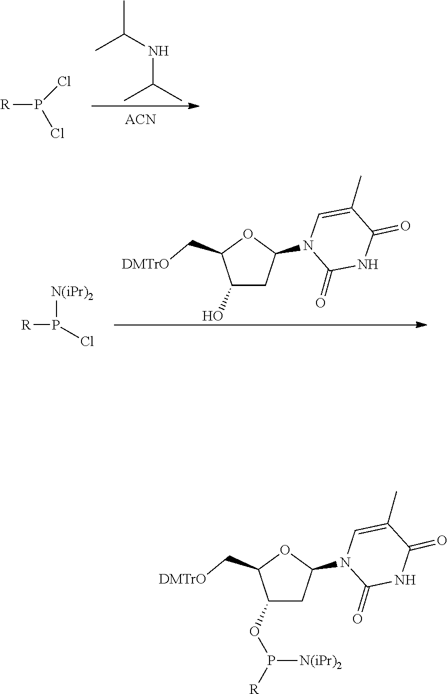

An appropriate dichlorophosphine is selected and reacted with diisopropylamine and then the product is reacted with a selected DMT protected nucleoside (for example thymidine). The resulting reactive phosphorus group will form the corresponding phosphonate internucleoside linkage (R-phosphonate in this example) when reacted with a free hydroxyl of a nucleoside or oligonucleotide, generally the 5′-hydroxyl, during routine oligonucleotide synthesis.

Secondary alkyl diisopropylaminophosphonamidite thymidine nucleosides were prepared wherein R is isopropyl and tertbutyl (spectra for each consistent with structure).

### Example 17

Synthesis of DMT protected tetrahydropyran diisopropylaminophosphoramidite nucleosides

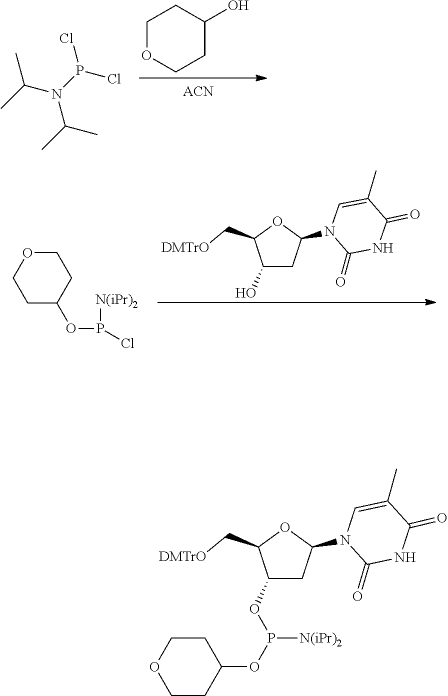

Substituted phosphates can be prepared by reacting diisopropylphosphoramidous dichloride the appropriate alcohol, and reacting the product with a protected nucleoside to provide the corresponding phosphoramidite.

A desired alcohol is selected and reacted with diisopropylphosphoramidous dichloride and then the product is reacted with a selected DMT protected nucleoside. The resulting reactive phosphorus group will form the corresponding phosphate internucleoside linkage (tetrahydropyran phosphate in this example) when reacted with a free hydroxyl of a nucleoside or oligonucleotide, generally the 5′-hydroxyl, during routine oligonucleotide synthesis.

This scheme was followed to also prepare the corresponding dimethylaminopropyl, isopropyl and ethyl substituted phosphate internucleoside linkages.

### Example 18

### Synthesis of Amide 3 Dimers

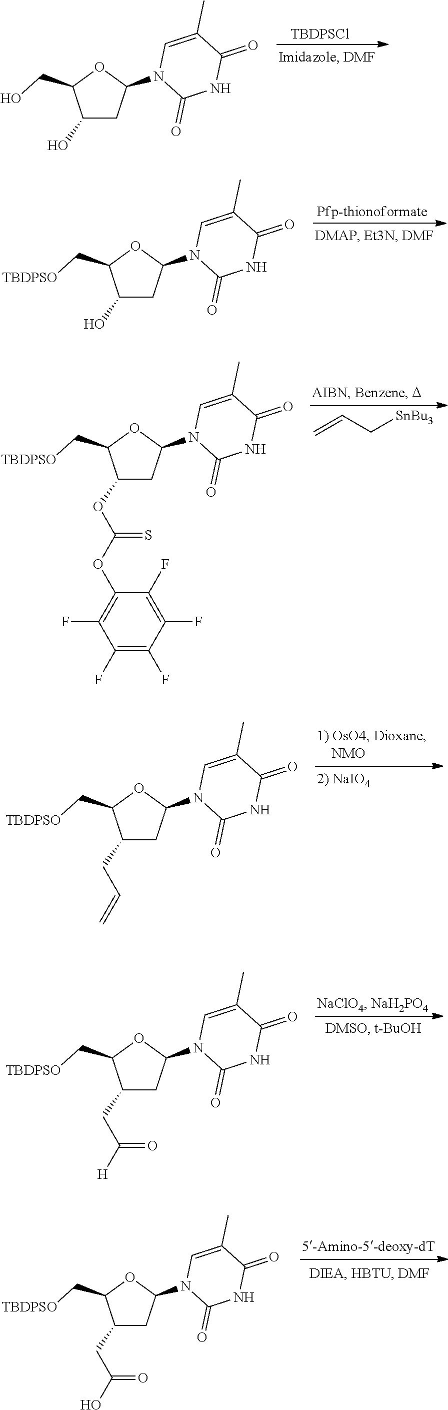

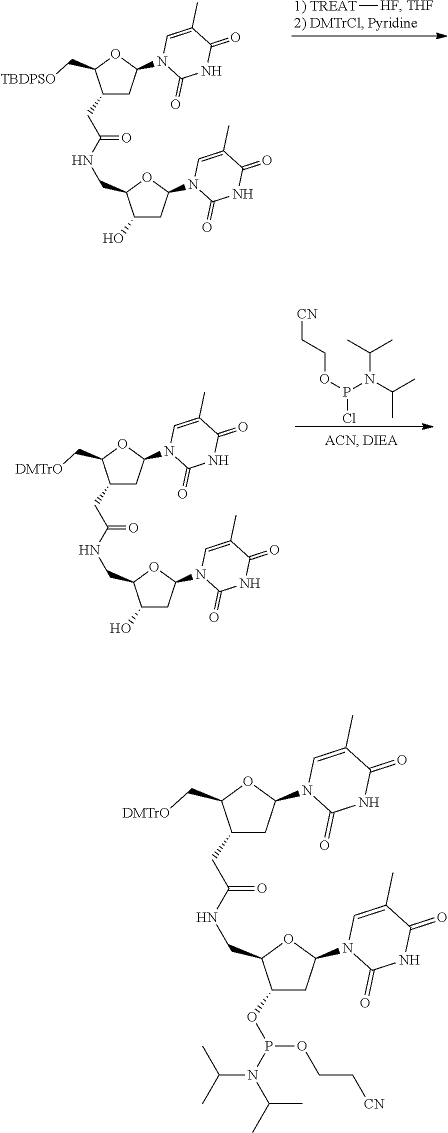

Amide 3 dimers were prepared as per the scheme illustrated above.

### Example 19

### Synthesis of Formacetal Dimers

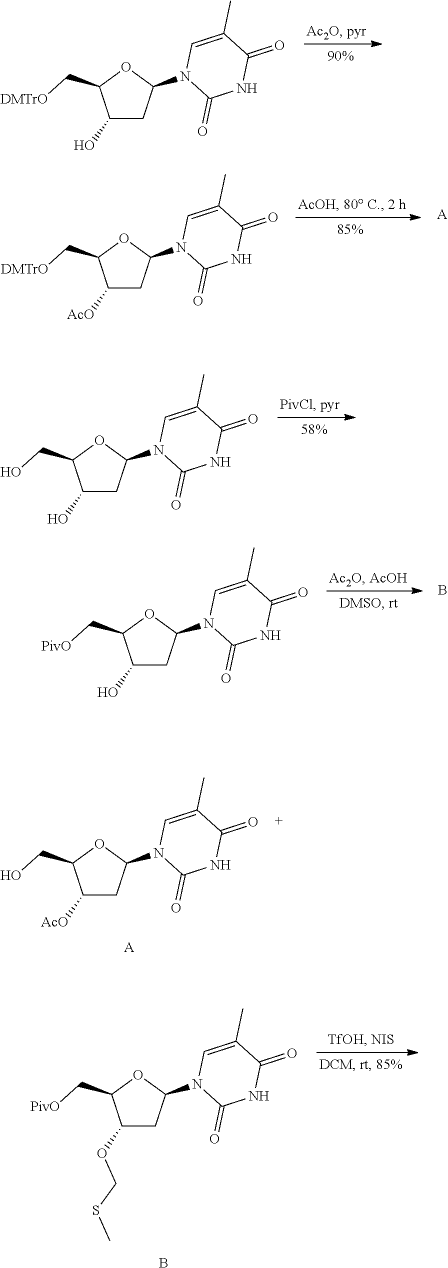

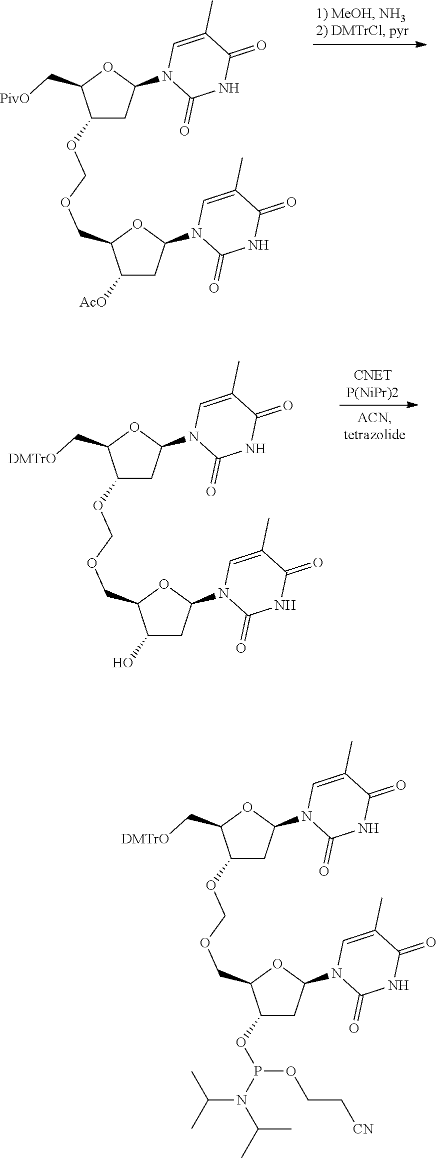

Formacetal dimers were prepared as per the scheme illustrated above.

### Example 20

### Synthesis of Sulfonamide Dimers

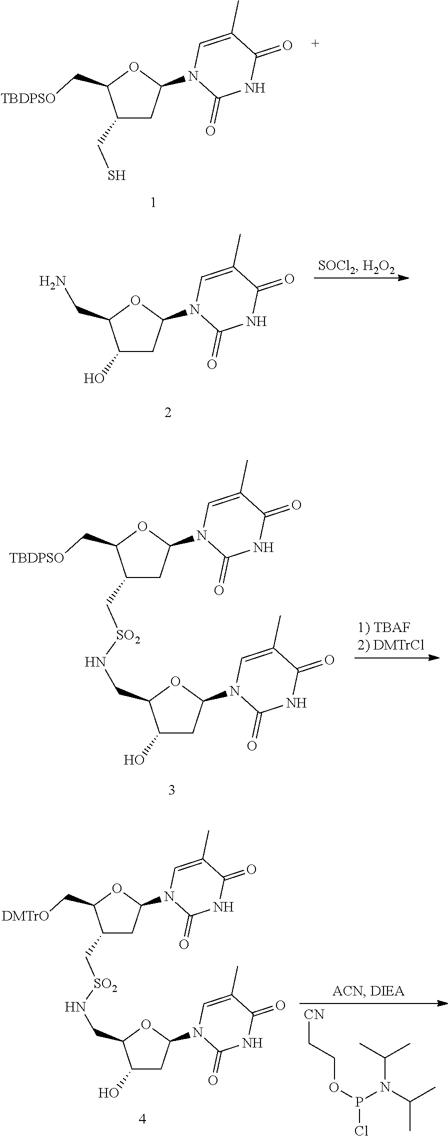

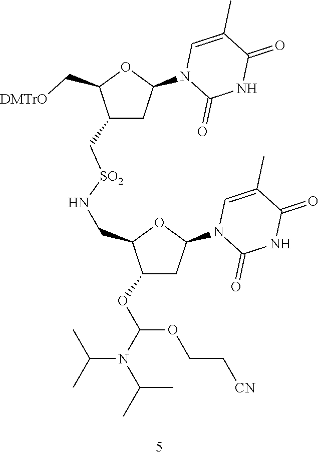

Compound 1 is prepared (as per Hutter et al., Helvetica Chimica Acta, 2002, 85, 2777) and treated with commercially available compound 2 as per published literature procedures (Bahrami et al., J Org Chem, 2009, 74, 9287) to give sulfonamide dimer 3. The silyl protecting group of is removed using tetrabutylammonium fluoride and the 5′-DMT group is added using dimethoxytrityl chloride in a suitable solvent. The tritylated compound is phosphitylated using standard methods to provide the phosphoramidite dimer Compound 5. Dimers containing any combination of the bases U, T, C, ^{Me}C, G, and A can be prepared in an analogous manner.

### Example 21

### Synthesis of Sulfinimide Dimers

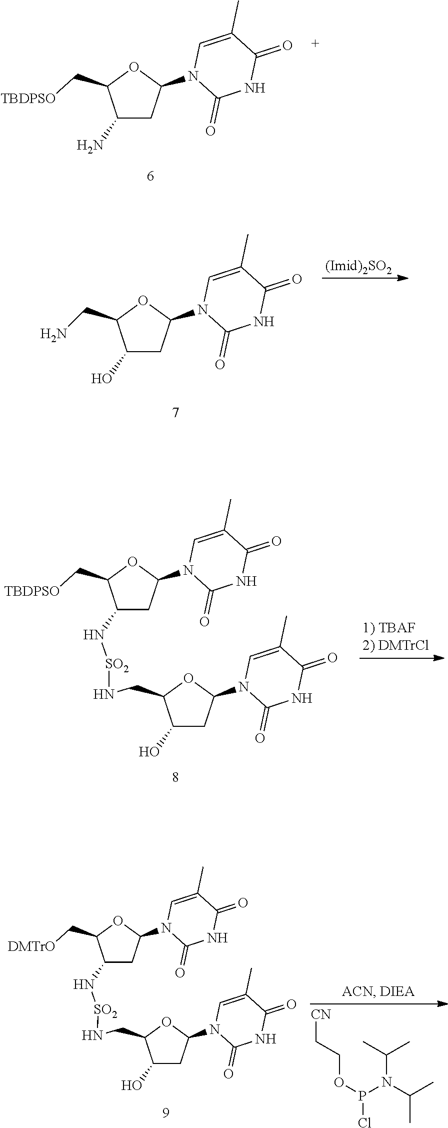

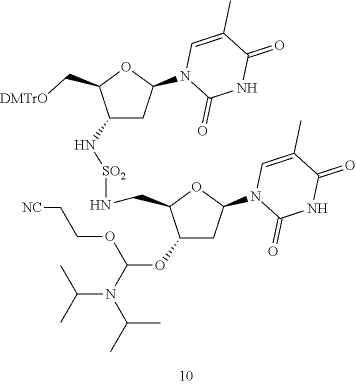

Commercially available compounds 6 and 7 are coupled using published methods (Beaudoin et al., J Org Chem, 2003, 68, 115) to give sulfonamide dimer, Compound 8. The silyl protecting groups of Compound 8 are then removed using tetrabutylammonium fluoride and the 5′-DMT group is added using dimethoxytrityl chloride in a suitable solvent. The tritylated compound is phosphitylated using standard methods to provide the phosphoramidite dimer Compound 10. Dimers containing any combination of the bases U, T, C, ^{Me}C, G, and A can be prepared in an analogous manner.

### Example 22

### Stability and Cleavage Patterns of Modified Oligonucleotides (RNA/ASO Duplexes) Subjected to RNaseH 1 Treatment

Modified oligonucleotides were designed based on the control oligonucleotide ISIS 558807, having a 3/10/3 gapmer motif wherein each internucleoside linkage is a phosphorothioate, the gap region contains ten β-D-2′-deoxyribonucleosides and each wing contains 3 cEt bicyclic nucleosides. Modified internucleoside linkages were positioned at various positions within gap of the oligonucleotides as illustrated below. The resulting modified oligonucleotides (ASOs) were hybridized to complementary RNA strands to provide RNA/ASO duplexes that were then treated with Human RNase H1.

Human RNase H1 (1:100 dilution) was prepared by adding Human RNase H1 (1.0 μL) to RNase H1 dilution buffer (72 (RNase H1 dilution buffer: glycerol 30%; 20 mM Tris pH7.5; 50 mM NaCl) and RNAseOUT (8 μL). The dilution was allowed to incubate for 1 hour prior to use.

RNA/ASO duplexes were prepared by heating a buffered solution of each of the modified oligonucleotides (400 nM) listed in the table below with the complementary RNA (IDT, 200 nm unlabeled and 1 nm 5′-³²P labeled) to 90° C. for 2 minutes. The buffered solution is prepared having 20 mM Tris pH 7.5; 50 mM NaCl; 2 mM MgCl; 0.2 mM TCEP; and 2 μL RNAseOUT.

To each of the RNA/ASO duplexes (20 μL) is added the Human RNase H1 solution (1 μL) in a heat block at 37° C. for 30 minutes. The samples are then quenched with urea (20 μL, 8M) and heated to 90° C. for 2 minutes. The antisense oligonucleotides are shown in Table 1 below.

TABLE 1

| SEQ ID | | |
|---|---|---|
| NO./ | | |
| ISIS NO. | Composition (5′ to 3′) | Linkage |
| 04/IDT | UAAUGUGAGAACAUGC | RNA (complement) |
| 03/558807 | Gₖ^{m}CₖAₖTGTT^{m}CT^{m}CA^{m}CATₖTₖAₖ | full PS (parent) |
| 03/857528 | Gₖ^{m}CₖAₖTGTmT^{m}CT^{m}CA^{m}CATₖTₖAₖ | THP phosphate/PS |
| 03/857529 | Gₖ^{m}CₖAₖTGTvT^{m}CT^{m}CA^{m}CATₖTₖAₖ | isopropylphosphate/PS |
| 03/857530 | Gₖ^{m}CₖAₖTGT_{IIa}T^{m}CT^{m}CA^{m}CATₖTₖAₖ | isobutylphosphonate |
| 03/857505 | Gₖ^{m}CₖAₖTGT_{Ia}T^{m}CT^{m}CA^{m}CATₖTₖAₖ | isopropylphosphonate |
| 03/883401 | Gₖ^{m}CₖAₖTGT_{VIII}T^{m}CT^{m}CA^{m}CATₖTₖAₖ | amide-3/PS |
| 03/883521 | Gₖ^{m}CₖAₖTGT_{IX}T^{m}CT^{m}CA^{m}CATₖTₖAₖ | formacetal/PS |
| 03/857532 | Gₖ^{m}CₖAₖTGTT_{III}^{m}CT^{m}CA^{m}CATₖTₖAₖ | THP phosphate/PS |
| 03/857533 | Gₖ^{m}CₖAₖTGTTv^{m}CT^{m}CA^{m}CATₖTₖAₖ | isopropylphosphate/PS |
| 03/857538 | Gₖ^{m}CₖAₖTGT_{III}T_{III}^{m}CT^{m}CA^{m}CATₖTₖAₖ | THP phosphate/PS |
| 03/857529 | Gₖ^{m}CₖAₖTGTvTv^{m}CT^{m}CA^{m}CATₖTₖAₖ | isopropylphosphate/PS |

Between adjacent nucleosides subscripts “Ia”, “IIa”, “III”, “V”, “VIII” and “IX” indicate a modified internucleoside linkage as depicted below and all other internucleoside linkages are phosphorothioate. Each nucleoside followed by a subscript “k” is a bicyclic nucleoside having a 4′-CH((S)—CH₃))—O-2′ bridging group (cEt) and all other nucleosides are 2′-deoxyribonucleosides except for the complementary RNA (SEQ ID NO: 4, purchased from IDT). Each “^{m}C” indicates that this nucleoside comprises a 5-methyl cytosine nucleobase.

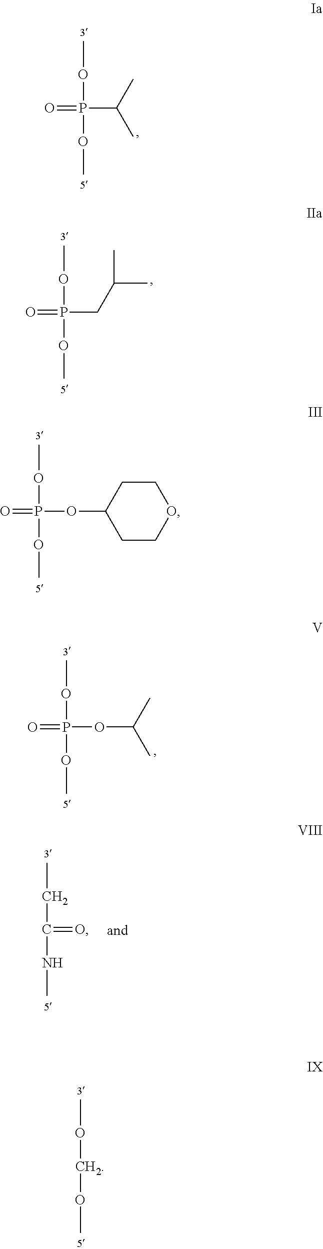

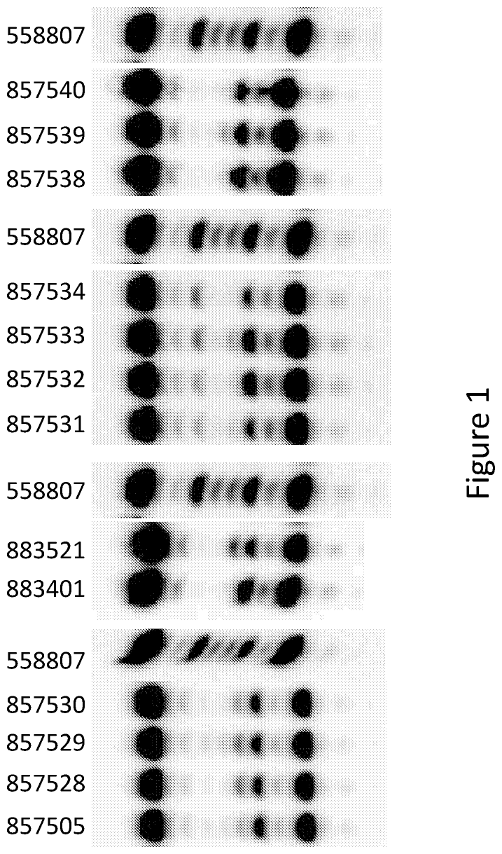

The cleavage products were resolved on polyacrylamide gel shown in FIG. 1. The parent oligo is the same and is only shown twice for the 4 different gels that were run. The cleavage pattern for the parent oligo was the same on each gel.

### Example 23

### Thermal Stability Assay

A series of modified oligomeric compounds were evaluated in a thermal stability (Tₘ) assay. A Cary 100 Bio spectrophotometer with the Cary Win UV Thermal program was used to measure absorbance vs. temperature. For the Tₘ experiments, oligomeric compounds were prepared at a concentration of 8 μM in a buffer of 100 mM Na+, 10 mM phosphate and 0.1 mM EDTA (pH 7). The concentration of the oligonucleotides was determined at 85° C. The concentration of each oligomeric compound was 4 μM after mixing of equal volumes of test oligomeric compound and complimentary RNA strand. Oligomeric compounds were hybridized with the complimentary RNA strand by heating the duplex to 90° C. for 5 minutes followed by cooling to room temperature. Using the spectrophotometer, Tₘ measurements were taken by heating the duplex solution at a rate of 0.5° C./min in cuvette starting @ 15° C. and heating to 85° C. Tₘ values were determined using Vant Hoff calculations (A₂₆₀ vs temperature curve) using non self-complementary sequences where the minimum absorbance which relates to the duplex and the maximum absorbance which relates to the non-duplex single strand are manually integrated into the program. The oligomeric compounds were hybridized to complementary RNA (ISIS 606581). The results are presented in Table 2 below.

TABLE 2

| SEQ ID NO./ | | | |
|---|---|---|---|
| ISIS #/ | Composition | ΔTm | |
| *ION# | (5′ to 3′) | ° C. | Linkage |
| 02/606581 | UCGAGAACAUCC | n/a | PO/RNA |
| | | | compl. |
| 01/606339 | GGATGTTCTCGA | 0.0 | PO/DNA |
| | | (Tm | std. |
| | | 48.4) | |
| 01/802510 | GGAT_{I}GTTCTCGA | -2.9 | Cyclohexyl |
| 01/802511 | GGATGT_{I}TCTCGA | -2.7 | Cyclohexyl |
| 01/802512 | GGATGTT_{I}CTCGA | -1.5 | Cyclohexyl |
| 01/802513 | GGATGTTCT_{I}CGA | -2.1 | Cyclohexyl |
| 01/802514 | GGATGT_{I}T_{I}CTCGA | -2.4 | Cyclohexyl |
| 01/948451* | GGAT_{Ia}GTTCTCGA | -1.0 | isopropyl |
| 01/948452* | GGATGT_{Ia}TCTCGA | -1.5 | isopropyl |
| 01/948453* | GGATGTT_{Ia}CTCGA | -0.4 | isopropyl |
| 01/948454* | GGATGTTCT_{Ia}CGA | -0.6 | isopropyl |
| 01/948455* | GGATGT_{Ia}T_{Ia}CTCGA | -1.3 | isopropyl |
| 01/644785 | GGAT_{II}GTTCTCGA | -3.7 | Phenyl |
| 01/644787 | GGATGT_{II}TCTCGA | -3.2 | Phenyl |
| 01/644786 | GGATGTT_{II}CTCGA | -4.1 | Phenyl |
| 01/644789 | GGATGTTCT_{II}CGA | -4.0 | Phenyl |
| 01/644788 | GGATGT_{II}T_{II}CTCGA | -4.2 | Pheny |
| 01/948456* | GGAT_{IIa}GTTCTCGA | -1.5 | isobutyl |
| 01/948457* | GGATGT_{IIa}TCTCGA | -2.0 | isobutyl |
| 01/948458* | GGATGTT_{IIa}CTCGA | -1.5 | isobutyl |
| 01/948459* | GGATGTTCT_{IIa}CGA | -2.1 | isobutyl |
| 01/948460* | GGATGT_{IIa}T_{IIa}CTCGA | -2.1 | isobutyl |
| 01/636964 | GGAT_{IIb}GTTCTCGA | -0.9 | Propyl |
| 01/636965 | GGATGT_{IIb}TCTCGA | -3.0 | Propyl |
| 01/636966 | GGATGTT_{IIb}CTCGA | -1.7 | Propyl |
| 01/636967 | GGATGTTCT_{IIb}CGA | -2.4 | Propyl |
| 01/636968 | GGATGT_{IIb}T_{IIb}CTCGA | -2.4 | Propyl |
| 01/636964 | GGAT_{IIc}GTTCTCGA | -1.4 | Pentyl |
| 01/636965 | GGATGT_{IIc}TCTCGA | -2.1 | Pentyl |
| 01/636966 | GGATGTT_{IIc}CTCGA | -1.8 | Pentyl |
| 01/636967 | GGATGTTCT_{IIc}CGA | -1.2 | Pentyl |
| 01/636968 | GGATGT_{IIc}T_{IIc}CTCGA | NA | Pentyl |
| 01/948461* | GGAT_{III}GTTCTCGA | -1.2 | THP |
| 01/948462* | GGATGT_{III}TCTCGA | -0.5 | THP |
| 01/948463* | GGATGTT_{III}CTCGA | -0.4 | THP |
| 01/948464* | GGATGTTCT_{III}CGA | -2.1 | THP |
| 01/948465* | GGATGT_{III}T_{III}CTCGA | -2.1 | THP |
| 01/948466* | GGAT_{IV}GTTCTCGA | -1.7 | OEt |
| 01/948467* | GGATGT_{IV}TCTCGA | -1.9 | OEt |
| 01/948468* | GGATGTT_{IV}CTCGA | -1.2 | OEt |
| 01/948469* | GGATGTTCT_{IV}CGA | -2.4 | OEt |
| 01/948470* | GGATGT_{IV}T_{IV}CTCGA | -2.2 | OEt |
| 01/948471* | GGAT_{V}GTTCTCGA | -1.2 | OiPr |
| 01/948472* | GGATGT_{V}TCTCGA | -2.0 | OiPr |
| 01/948473* | GGATGTT_{V}CTCGA | -1.0 | OiPr |
| 01/948474* | GGATGTTCT_{V}CGA | -0.6 | OiPr |
| 01/948481* | GGATGT_{V}T_{V}CTCGA | -2.2 | OiPr |
| 01/948476* | GGAT_{VI}GTTCTCGA | -1.5 | DMAP |
| 01/948477* | GGATGT_{VI}TCTCGA | -2.1 | DMAP |
| 01/948478* | GGATGTT_{VI}CTCGA | -1.8 | DMAP |
| 01/948479* | GGATGTTCT_{VI}CGA | -2.6 | DMAP |
| 01/948480* | GGATGT_{VI}T_{VI}CTCGA | NA | DMAP |

Between adjacent nucleosides subscripts “I”, “II”, “III”, “IV”, “V” and “VI” indicate a modified internucleoside linkage as depicted below and all other internucleoside linkages are phosphodiester. All nucleosides are 2′-deoxyribonucleosides except for the complementary RNA (SEQ ID NO: 02, ISIS NO.: 606581).

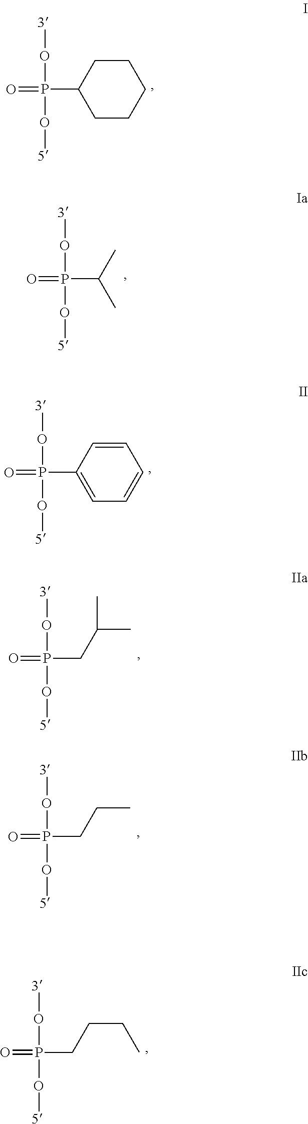

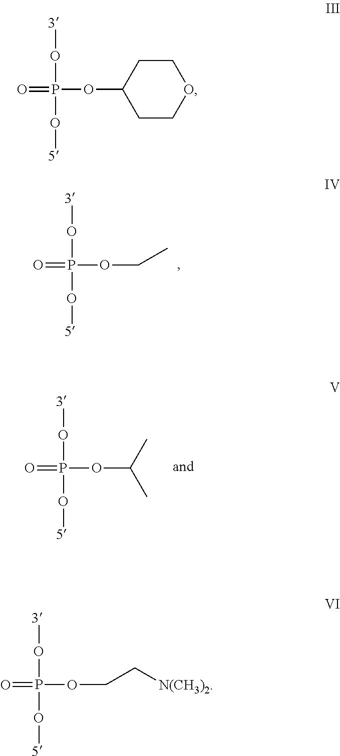

### Example 24

### Modified Oligonucleotides Targeting CXCL12 In Vitro Study

Modified oligonucleotides were designed based on the control oligonucleotide ISIS 558807, having a 3/10/3 gapmer motif wherein each internucleoside linkage is a phosphorothioate, the gap region contains ten β-D-2′-deoxyribonucleosides and each wing contains 3 cEt bicyclic nucleosides (Table 2). Modified internucleoside linkages (1 or 2) were positioned at various positions within gap of the oligonucleotides as illustrated below. The resulting modified oligonucleotides were tested for their ability to inhibit CXCL12 (Chemokine ligand 12), Raptor, Fars2 and Ppp3Ca mRNA expression levels. The potency of the modified oligonucleotides was evaluated and compared to the control oligonucleotide.

The modified oligonucleotides were tested in vitro in mouse b.END cells by electroporation. Cells at a density of 20,000 cells per well are transfected using electroporation with 0.027, 0.082, 0.25, 0.74, 2.22, 6.67 and 20 uM concentrations of each of the oligonucleotides listed below. After a treatment period of approximately 24 hours, RNA is isolated from the cells and mRNA levels are measured by quantitative real-time PCR and the CXCL12 mRNA and Raptor mRNA levels are adjusted according to total RNA content, as measured by RIBOGREEN®.

TABLE 3

| SEQID NO./ | | |
|---|---|---|
| ISIS NO. | Composition (5′ to 3′) | Linkage |
| 03/558807 | Gₖ^{m}CₖAₖTGTT^{m}CT^{m}CA^{m}CATₖTₖAₖ | full PS |
| 03/857505 | Gₖ^{m}CₖAₖTGT_{Ia}T^{m}CT^{m}CA^{m}CATₖTₖAₖ | iPr (x1)/PS |
| 03/857530 | Gₖ^{m}CₖAₖTGT_{IIa}T^{m}CT^{m}CA^{m}CATₖTₖAₖ | iBu (x1)/PS |
| 03/857528 | Gₖ^{m}CₖAₖTGT_{III}T^{m}CT^{m}CA^{m}CATₖTₖAₖ | THP (x1)/PS |
| 03/857529 | Gₖ^{m}CₖAₖTGTvT^{m}CT^{m}CA^{m}CATₖTₖAₖ | OiPr (x1)/PS |
| 03/883401 | Gₖ^{m}CₖAₖTGT_{VIII}T^{m}CT^{m}CA^{m}CATₖTₖAₖ | amide 3 (x1)/PS |
| 03/883521 | Gₖ^{m}CₖAₖTGT_{IX}T^{m}CT^{m}CA^{m}CATₖTₖAₖ | formacetal (x1)/PS |
| 03/857531 | Gₖ^{m}CₖAₖTGTT_{Ia}^{m}CT^{m}CA^{m}CATₖTₖAₖ | iPr (x1)/PS |
| 03/857534 | Gₖ^{m}CₖAₖTGTT_{IIa}^{m}CT^{m}CA^{m}CATₖTₖAₖ | iBu (x1)/PS |
| 03/857532 | Gₖ^{m}CₖAₖTGTT_{III}^{m}CT^{m}CA^{m}CATₖTₖAₖ | THP (x1)/PS |
| 03/857533 | Gₖ^{m}CₖAₖTGTTv^{m}CT^{m}CA^{m}CATₖTₖAₖ | OiPr (x1)/PS |
| 03/857537 | Gₖ^{m}CₖAₖTGT_{Ia}T_{Ia}^{m}CT^{m}CA^{m}CATₖTₖAₖ | iPr (x2)/PS |
| 03/857540 | Gₖ^{m}CₖAₖTGT_{IIa}T_{IIa}^{m}CT^{m}CA^{m}CATₖTₖAₖ | iBu (x2)/PS |
| 03/857538 | Gₖ^{m}CₖAₖTGT_{III}T_{III}^{m}CT^{m}CA^{m}CATₖTₖAₖ | THP (x2)/PS |
| 03/857539 | Gₖ^{m}CₖAₖTGTvTv^{m}CT^{m}CA^{m}CATₖTₖAₖ | OiPr (x2)/PS |

Between adjacent nucleosides subscripts “III”, “V”, “VIII” and “IX” indicate a modified internucleoside linkage as depicted below and all other internucleoside linkages are phosphorothioate. Each nucleoside followed by a subscript “k” is a bicyclic nucleoside having a 4′-CH((S)—CH₃))—O-2′ bridging group (cEt) and all other nucleosides are 2′-deoxyribonucleosides. Each “^{m}C” indicates that this nucleoside comprises a 5-methyl cytosine nucleobase.

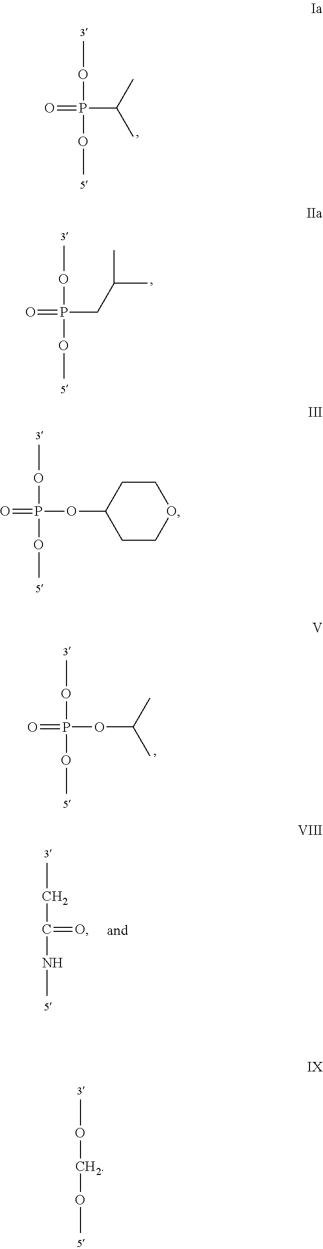

The half maximal inhibitory concentration (IC₅₀) of each oligonucleotide listed above was calculated by plotting the concentration of oligonucleotide versus the percent inhibition of CXCL12 mRNA or Raptor mRNA expression achieved at each concentration, and noting the concentration of oligonucleotide at which 50% inhibition of CXCL12 mRNA expression is achieved compared to the control. The results are presented in Table 4 below:

TABLE 4

| | | Raptor % | Fars2 % | Ppp3Ca % |
|---|---|---|---|---|
| SEQ ID NO./ | IC₅₀ (μM) | Control | Control | Control |
| ISIS NO. | CXCL12 | (4 μM) | (4 μM) | (4 μM) |
| 03/558807 | 0.17 | 47 | 65 | 73 |
| 03/857505 | 0.15 | 82 | 83 | 68 |
| 03/857530 | 0.32 | 87 | 103 | 107 |
| 03/857528 | 0.23 | 110 | 89 | 85 |
| 03/857529 | 1.09 | 74 | 79 | 76 |
| 03/883401 | 30 | 65 | 59 | 80 |
| 03/883521 | 0.40 | 94 | 92 | 83 |
| 03/857531 | 0.27 | 99 | 87 | 78 |
| 03/857534 | 0.12 | 57 | 89 | 74 |
| 03/857532 | 0.16 | 69 | 77 | 73 |
| 03/857533 | 0.10 | 61 | 97 | 76 |
| 03/857537 | 1.4 | 82 | 83 | 68 |
| 03/857540 | 0.48 | 65 | 59 | 80 |
| 03/857538 | 0.33 | 110 | 89 | 85 |
| 03/857539 | 0.13 | 74 | 79 | 76. |

### Example 25

### Modified Oligonucleotides Targeting CXCL12 In Vivo Study

Modified oligonucleotides were designed based on ISIS 558807, having a 3/10/3 gapmer motif wherein each internucleoside linkage is a phosphorothioate, the gap region contains ten β-D-2′-deoxyribonucleosides and each wing contains 3 cEt bicyclic nucleosides. Each modified oligonucleotide has a modified internucleoside linkage positioned between nucleosides 3 and 4 counting from the 5′-gap junction (not including the 3 cEt modified nucleosides in the 5′-wing) as illustrated below. Each of the modified oligonucleotides is conjugated with a THA conjugate group at the 3′-end as illustrated below. The oligonucleotides were evaluated for reduction in CXCL12 (Chemokine ligand 12) mRNA expression levels in vivo. The transaminase levels (ALT and AST) for each dose were also measured.

Six week old BALB/C mice (purchased from Charles River) were injected subcutaneously once at dosage 0.2, 0.6, 1.8 or 50 mg/kg with the modified oligonucleotides shown below or with saline control. Each treatment group consisted of 3 animals. The mice were sacrificed 72 hours following administration, and organs and plasma were harvested for further analysis.

TABLE 5

| SEQ ID | | |
|---|---|---|
| NO./ | | |
| ION NO. | Composition (5′ to 3′) | Linkage |
| 03/855156* | Gₖ^{m}CₖAₖTGTT^{m}CT^{m}CA^{m}CATₖTₖAₖ-THA | full PS |
| 03/895566 | Gₖ^{m}CₖAₖTGT_{Ia}T^{m}CT^{m}CA^{m}CATₖTₖAₖ-THA | iPr |
| 03/895567 | Gₖ^{m}CₖAₖTGT_{III}T^{m}CT^{m}CA^{m}CATₖTₖAₖ-THA | THP |
| 03/895568 | Gₖ^{m}CₖAₖTGTvT^{m}CT^{m}CA^{m}CATₖTₖAₖ-THA | OiPr |
| 03/895569 | Gₖ^{m}CₖAₖTGT_{IIa}T^{m}CT^{m}CA^{m}CATₖTₖAₖ-THA | iBu |
| 03/895570 | Gₖ^{m}CₖAₖTGT_{IX}T^{m}CT^{m}CA^{m}CATₖTₖAₖ-THA | formacetal |
| 03/913196 | Gₖ^{m}CₖAₖTGT_{VIII}T^{m}CT^{m}CA^{m}CATₖTₖAₖ-THA | amide 3 |
| 03/920046 | Gₖ^{m}CₖAₖTGT_{XIII}T^{m}CT^{m}CA^{m}CATₖTₖAₖ-THA | TANA |

* Oligonucleotide was run in a separate assay and is shown for comparison and is ISIS 855156 not an ION #. Between adjacent nucleosides subscripts “Ia”, “IIa”, “III”, “V”, “VIII”, “IX”, and “XIII” indicate a modified internucleoside linkage as depicted below and all other internucleoside linkages are phosphorothioate. Each nucleoside followed by a subscript “k” is a bicyclic nucleoside having a 4′-CH((S)—CH₃))—O-2′ bridging group (cEt) and all other nucleosides are 2′-deoxyribonucleosides. Each “NC” indicates that this nucleoside comprises a 5-methyl cytosine nucleobase.

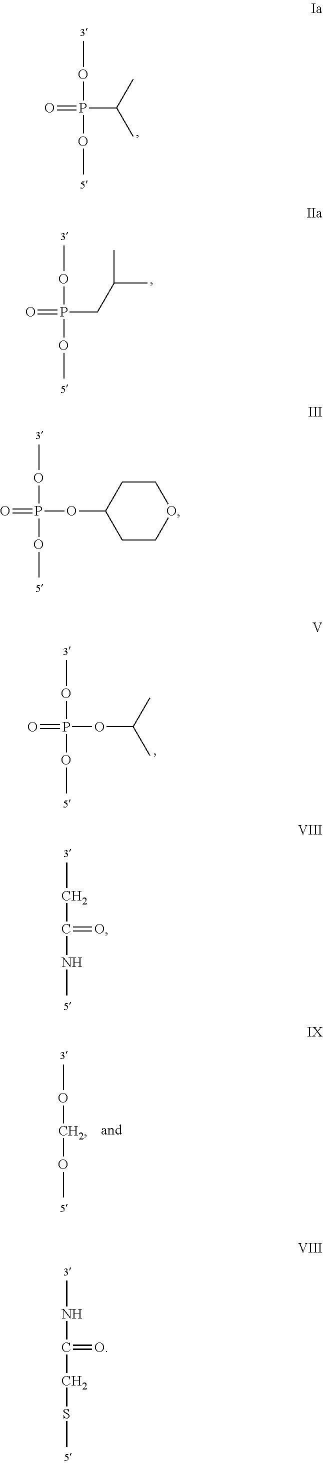

Each modified oligonucleotide in the study includes a 3′-THA conjugate group which is attached to the 3′-oxygen of the oligomeric compound. The 3′-THA conjugate group is illustrated below wherein the phosphate group is attached to the 3′-oxygen atom:

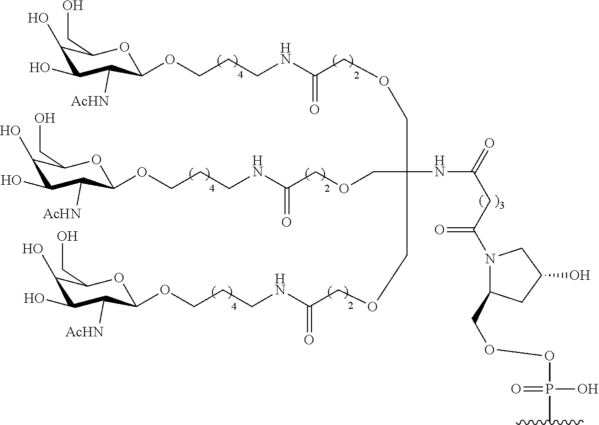

Liver tissues were homogenized and mRNA levels were quantitated using real-time PCR and normalized to RIBOGREEN as described herein. Plasma chemistry markers such as liver transaminase levels, alanine aminotransferase (ALT) in serum were measured relative to saline injected mice.

The ED₅₀s values were calculated by plotting the concentrations of oligonucleotides used versus the percent inhibition of CXCL12 mRNA expression achieved at each concentration, and noting the concentration of oligonucleotide at which 50% inhibition of CXCL12 mRNA expression was achieved compared to the control.

The results are presented in Table 6 below:

TABLE 6

| SEQ ID | | | | | | |
|---|---|---|---|---|---|---|
| NO./ISIS | Dose | % | | | | |
| NO | mg/kg | control | ED₅₀ | ALT | AST | Linkage |
| Saline | | 100 | | 26 | 56 | |
| 03/855156* | 0.21 | 81 | | 28 | 64 | full PS |
| | 0.62 | 63 | | 37 | 76 | |
| | 1.85 | 45 | | 2359 | 3404 | |
| | 5.56 | 31 | | 4298 | 5656 | |
| 03/895566 | 0.2 | 68 | 0.85 | 30 | 50 | iPr |
| | 0.6 | 55 | | 27 | 46 | |
| | 1.8 | 42 | | 28 | 27 | |
| | 50 | 22 | | 24 | 40 | |
| 03/895567 | 0.2 | 59 | 0.38 | 32 | 60 | THP |
| | 0.6 | 50 | | 61 | 51 | |
| | 1.8 | 36 | | 26 | 49 | |
| | 50 | 18 | | 25 | 45 | |
| 03/895568 | 0.2 | 69 | 0.38 | 28 | 61 | OiPr |
| | 0.6 | 49 | | 29 | 59 | |
| | 1.8 | 37 | | 33 | 66 | |
| | 50 | 17 | | 38 | 56 | |
| 03/895569 | 0.2 | 72 | 0.59 | 27 | 60 | iBu |
| | 0.6 | 51 | | 23 | 44 | |
| | 1.8 | 41 | | 30 | 77 | |
| | 50 | 18 | | 28 | 49 | |
| 03/895570 | 0.2 | 68 | 0.46 | 33 | 54 | formacetal |
| | 0.6 | 50 | | 27 | 46 | |
| | 1.8 | 38 | | 24 | 43 | |
| | 50 | 17 | | 31 | 47 | |
| 03/913196 | 0.2 | 62 | 0.53 | 24 | 43 | amide 3 |
| | 0.6 | 48 | | 25 | 46 | |
| | 1.8 | 44 | | 23 | 41 | |
| | 50 | 19 | | 29 | 49 | |
| 03/920046 | 0.2 | 80 | 2.51 | 22 | 48 | TANA |
| | 0.6 | 58 | | 28 | 48 | |
| | 1.8 | 58 | | 25 | 48 | |
| | 50 | 25 | | 24 | 40. | |

1. An oligomeric compound comprising a gapped oligomeric compound comprising a contiguous sequence of linked monomer subunits having a 5′-region, a 3′-region and a gap region of from 6 to 14 contiguous β-D-2′-deoxyribonucleosides located between the 5′ and 3′-regions wherein the 5′ and 3′-regions each, independently, have from 2 to 8 contiguous modified nucleosides that are RNA-like that each adopt a 3′-endo conformational geometry when put into an oligomeric compound wherein each internucleoside linking group is, independently, a phosphodiester or a phosphorothioate internucleoside linking group providing that from 1 to about 3 internucleoside linking groups located in a gap junction and or the gap region is an internucleoside linking group having one of formulas I to XVI:

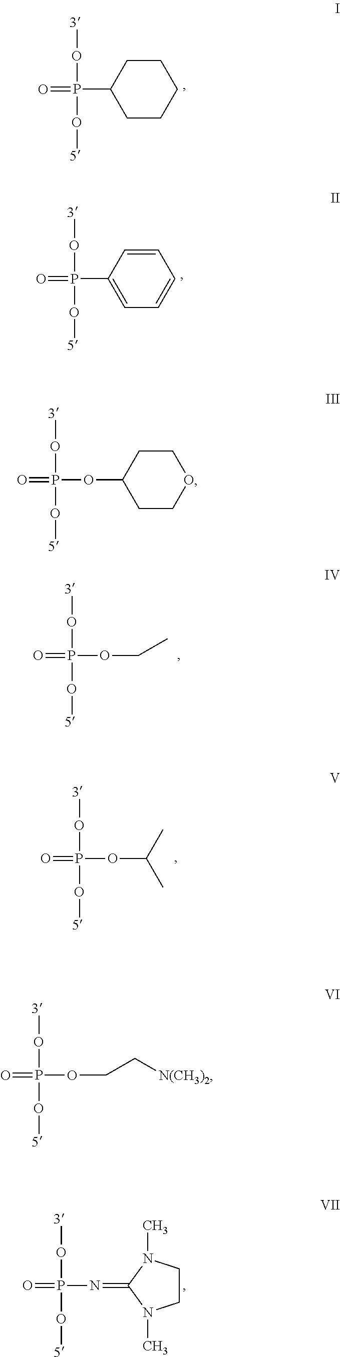

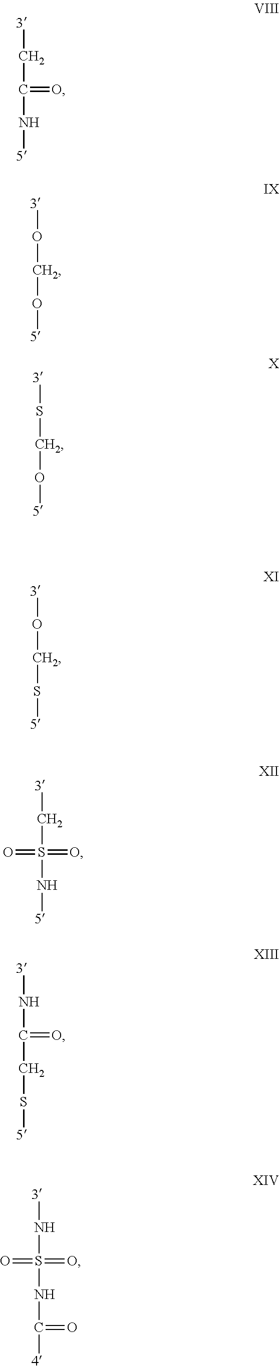

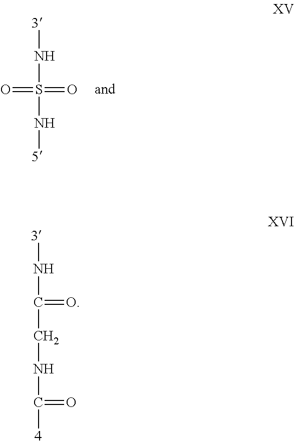

2. The gapped oligomeric compound of claim 1 comprising from 12 to 24 monomer subunits.

3. The gapped oligomeric compound of any of claim 1 or 2 comprising from 14 to 20 monomer subunits.

4. The gapped oligomeric compound of any of claims 1 to 4 wherein the gap region has 10 contiguous monomer subunits and the 5′ and 3′-regions each, independently, have 2, 3 or 5 contiguous monomer subunits.

5. The gapped oligomeric compound of any of claims 2 to 4 wherein the gap region has 10 contiguous monomer subunits and the 5′ and 3′-regions each have 5 contiguous monomer subunits.

6. The gapped oligomeric compound of any of claims 2 to 4 wherein the gap region has 10 contiguous monomer subunits and the 5′ and 3′-regions each have 3 contiguous monomer subunits.

7. The gapped oligomeric compound of any of claims 2 to 4 wherein the gap region has 10 contiguous monomer subunits and the 5′ and 3′-regions each have 2 contiguous monomer subunits.

8. The gapped oligomeric compound of any of claims 1 to 7 comprising from 1 to about 3 internucleoside linking groups located in a gap junction and or the gap region having Formula I.

9. The gapped oligomeric compound of any of claims 1 to 7 comprising from 1 to about 3 internucleoside linking groups located in a gap junction and or the gap region having Formula II.

10. The gapped oligomeric compound of any of claims 1 to 7 comprising from 1 to about 3 internucleoside linking groups located in a gap junction and or the gap region having Formula III.

11. The gapped oligomeric compound of any of claims 1 to 7 comprising from 1 to about 3 internucleoside linking groups located in a gap junction and or the gap region having one of formulas IV, V or VI.

12. The gapped oligomeric compound of any of claims 1 to 7 comprising from 1 to about 3 internucleoside linking groups located in a gap junction and or the gap region having formula VII.

13. The gapped oligomeric compound of any of claims 1 to 7 comprising from 1 to about 3 internucleoside linking groups located in a gap junction and or the gap region having one of formulas VIII, IX, X and XI.

14. The gapped oligomeric compound of any of claims 1 to 7 comprising from 1 to about 3 internucleoside linking groups located in a gap junction and or the gap region having formula XII.

15. The gapped oligomeric compound of any of claims 1 to 7 comprising from 1 to about 3 internucleoside linking groups located in a gap junction and or the gap region having formula XV.

16. The gapped oligomeric compound of any of claims 1 to 7 comprising from 1 to about 3 internucleoside linking groups located in a gap junction and or the gap region having one of formulas XIII, XIV and XVI.

17. The gapped oligomeric compound of any of claims 1 to 7 comprising from 1 to about 3 internucleoside linking groups located in a gap junction and or the gap region having formula XVI.

18. The gapped oligomeric compound of any of claims 1 to 17 having 2 internucleoside linking groups having one of formulas I to XVI.

19. The gapped oligomeric compound of any of claims 1 to 17 having 3 internucleoside linking groups having one of formulas I to XVI.

20. The gapped oligomeric compound of any of claims 1 to 17 having 2 internucleoside linking groups having one of formulas I to XVI located between nucleosides 1 and 3, 2 and 4, 3 and 5 or 4 and 6 counting from the 5′ gap junction.

21. The gapped oligomeric compound of any of claims 1 to 17 having 2 or 3 contiguous internucleoside linking groups having one of formulas I to XVI.

22. The gapped oligomeric compound of any of claims 1 to 21 wherein each internucleoside linking group having one of formulas I to XVI has the same formula.

23. The gapped oligomeric compound of any of claims 1 to 17 having 1 internucleoside linking group having one of formulas I to XVI.

24. The gapped oligomeric compound of any of claims 1 to 23 wherein the internucleoside linking groups in the 5′ and 3′-gap junctions are each, independently, a phosphodiester or a phosphorothioate internucleoside linking group.

25. The gapped oligomeric compound of any of claims 1 to 23 comprising an internucleoside linking group having one of formulas I to XVI located at the 5′-gap junction.

26. The gapped oligomeric compound of any of claims 1 to 23 comprising an internucleoside linking group having one of formulas I to XVI located at the 3′-gap junction.

27. The gapped oligomeric compound of any of claims 1 to 17 having one internucleoside linking group having one of formulas I to XVI located between nucleosides 1 and 2, 2 and 3 or between nucleosides 3 and 4 counting from the 5′ gap junction.

28. The gapped oligomeric compound of any of claims 1 to 27 wherein each internucleoside linking group other than internucleoside linking groups having one of formulas I to XVI is a phosphodiester internucleoside linking group.

29. The gapped oligomeric compound of any of claims 1 to 27 wherein each internucleoside linking group other than internucleoside linking groups having one of formulas I to XVI is a phosphorothioate internucleoside linking group.

30. The gapped oligomeric compound of any of claims 1 to 29 wherein each monomer subunit comprises a nucleobase independently selected from thymine, cytosine, 5-methylcytosine, adenine and guanine.

31. The gapped oligomeric compound of any of claims 1 to 30 wherein each modified nucleoside comprises a modified sugar moiety independently selected from a bicyclic nucleoside comprising a bicyclic furanosyl sugar moiety, a modified nucleoside comprising a furanosyl sugar moiety having at least one substituent group and a modified nucleoside comprising a sugar surrogate group.

32. The gapped oligomeric compound of any of claims 1 to 31 wherein each modified nucleoside is, independently, selected from a bicyclic nucleoside comprising a bicyclic furanosyl sugar moiety having a bridging group between the 4′ and 2′ carbon atoms of the furanosyl ring independently selected from 4′-CH₂—O-2′, 4′-(CH₂)₂—O-2′, 4′-CH(CH₃)—O-2′, 4′-CH₂—N(CH₃)—O-2′, 4′-CH₂—C(H)(CH₃)-2′ and 4′-CH₂—C(═CH₂)-2′ and a modified nucleoside comprising a ribofuranosyl sugar moiety having at least a 2′-substituent group independently selected from F, OCH₃, O(CH₂)₂—OCH₃ and OCH₂C(═O)—N(H)CH₃.

33. The gapped oligomeric compound of any of claims 1 to 32 wherein each of the modified nucleoside is, independently, selected from a bicyclic nucleoside comprising a bicyclic furanosyl sugar moiety having a 4′-CH₂—O-2′ or 4′-CH[(S)—(CH₃)]—O-2′ bridging group and a modified nucleoside comprising a ribofuranosyl sugar moiety having a 2′-O(CH₂)₂—OCH₃ substituent group.

34. The gapped oligomeric compound of any of claims 1 to 33 wherein each of the modified nucleosides is, independently, selected from a bicyclic nucleoside comprising a bicyclic furanosyl sugar moiety having a 4′-CH[(S)—(CH₃)]—O-2′ bridging group and a modified nucleoside comprising a ribofuranosyl sugar moiety having a 2′-O(CH₂)₂—OCH₃ substituent group.

35. The gapped oligomeric compound of any one of claims 1 to 31 wherein at least one of the modified nucleosides comprises a sugar surrogate.

36. The gapped oligomeric compound of any one of claims 1 to 35 wherein the modified nucleosides comprise 2 different types of sugar moieties.

37. The gapped oligomeric compound of any of claims 1 to 36 further comprising at least one 5′ or 3′-terminal group.

38. The gapped oligomeric compound of any of claims 1 to 37 having one optionally linked 5′ or 3′-conjugate group.

39. The gapped oligomeric compound of any of claims 1 to 37 having one optionally linked 3′-conjugate group.

40. The gapped oligomeric compound of any of claims 1 to 37 having one optionally linked 5′-conjugate group.

41. The gapped oligomeric compound of any of claims 38 to 40 wherein the conjugate group comprises a cell targeting moiety.

42. The gapped oligomeric compound of claim 41 wherein the cell targeting moiety has the formula:

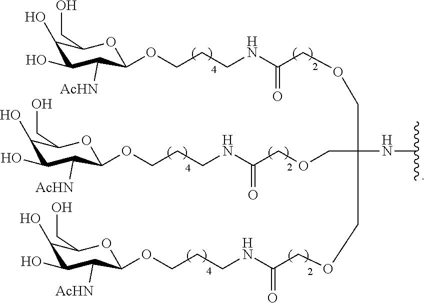

43. The gapped oligomeric compound of claim 41 wherein the cell targeting moiety has the formula:

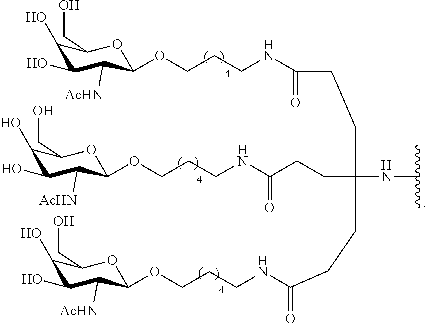

44. The gapped oligomeric compound of claim 41 wherein the cell targeting moiety has the formula:

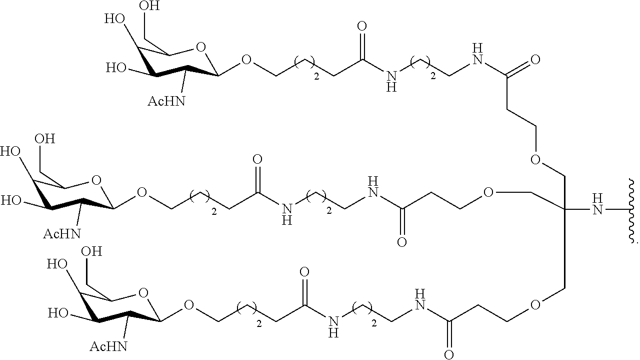

45. The gapped oligomeric compound of any of claims 41 to 45 wherein the attachment of the cell targeting moiety to the oligomeric compound includes a conjugate linker including a cleavable moiety having one of the formulas: 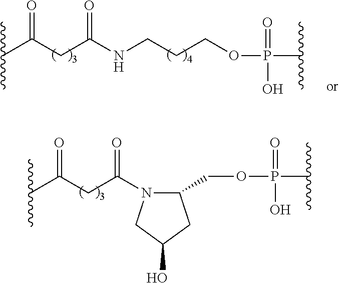
wherein the phosphate group is attached to the 3′ or 5′-terminal oxygen atom of the gapped oligomeric compound.

46. The gapped oligomeric compound of claim 41 wherein the conjugate group has the formula:

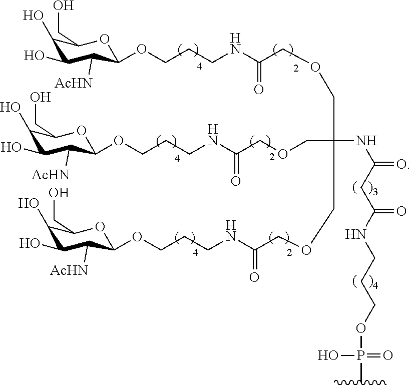

47. The gapped oligomeric compound of claim 46 wherein the conjugate group is attached to the 5′-terminal oxygen atom of the oligomeric compound.

48. The gapped oligomeric compound of claim 41 wherein the conjugate group has the formula:

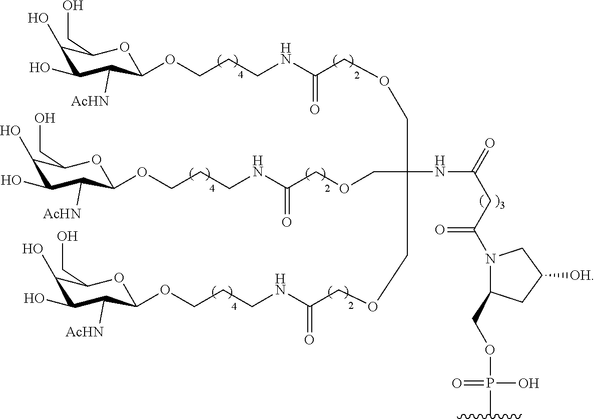

49. The gapped oligomeric compound of claim 48 wherein the conjugate group is attached to the 3′-terminal oxygen atom of the oligomeric compound.

50. A method of inhibiting gene expression comprising contacting one or more cells, a tissue or an animal with a gapped oligomeric compound of any of claims 1 to 49 wherein said oligomeric compound is complementary to a target RNA.

51. The method of claim 50 wherein said cells are in a human.

52. The method of claim 50 wherein said target RNA is human mRNA.

53. The method of claim 50 wherein said target RNA is cleaved thereby inhibiting its function.

54. An in vitro method of inhibiting gene expression comprising contacting one or more cells or a tissue with a gapped oligomeric compound of any one of claims 1 to 49.

55. A gapped oligomeric compound for use in an in vivo method of inhibiting gene expression said method comprising contacting one or more cells, a tissue or an animal with a gapped oligomeric compound of any of claims 1 to 49.

56. A gapped oligomeric compound of any one of claims 1 to 49 for use in medical therapy.
